(19) United States
(12) Patent Application Publication
(10) Pub. No.: US 2024/0368617 A1
(43) Pub. Date: Nov. 7, 2024

(54) NPR1 VARIANT TO ENHANCE PLANT RESISTANCE TO BIOTIC AND ABIOTIC STRESSES AND METHOD THEREOF

(71) Applicant: Duke University, Durham, NC (US)

(72) Inventors: Xinnian Dong, Durham, NC (US); Pei Zhou, Durham, NC (US); Raul Zavaliev, Durham, NC (US)

(21) Appl. No.: 18/652,899

(22) Filed: May 2, 2024

Related U.S. Application Data
(60) Provisional application No. 63/463,338, filed on May 2, 2023.

(51) Int. Cl. C12N 15/82; C07K 14/415; C12N 9/22; C12N 15/11; C12Q 1/6851; C12Q 1/6895

(52) U.S. Cl. CPC C12N 15/8271; C07K 14/415; C12N 9/22; C12N 15/11; C12N 15/8213; C12Q 1/6851; C12Q 1/6895; C12N 2310/20; C12Q 2600/13; C12Q 2600/158

(57) ABSTRACT

Described are NPR1 variants, nucleic acids encoding the NPR1 variants, and genetically modified plants expressing the NPR1 variants. Also described are methods of using nucleic acids encoding the NPR1 variants to genetically modify plants. The genetically modified plants exhibit increase resistance to biotic and abiotic stresses without compromising plant growth.

### CROSS-REFERENCE TO RELATED APPLICATIONS

This application claims the benefit of U.S. Provisional Application No. 63/463,338, filed May 2, 2023, which is incorporated herein by reference.

### STATEMENT REGARDING FEDERALLY SPONSORED RESEARCH OR DEVELOPMENT

This invention was made with Government support under Federal Grant nos. R35 GM118036, GM145026, and GM115355 awarded by the National Institutes of Health National Institute of General Medical Sciences (NIH/NIGMS). The Federal Government has certain rights to this invention.

### SEQUENCE LISTING

The Sequence Listing written in file DU8135_SeqListing.xml is 80 kilobytes in size, was created Apr. 30, 2024, and is hereby incorporated by reference.

### BACKGROUND

The Nonexpressor of Pathogenesis-Related Genes 1 (NPR1) gene is a positive regulator of the salicylic acid (SA)-mediated systemic acquired resistance (SAR) in plants. Overexpression of Arabidopsis NPR1 can enhance disease resistance against a variety of pathogens and stresses. However, this strategy often results in retardation in plant growth and reproduction dampening the potential for using NPR1 in agricultural applications. Thus, there is an ongoing need for improved systems and methods for engineered NPR1.

### SUMMARY

Described are genetically modified plants comprises a nucleic acid encoding a npr1(SAL) protein. The nucleic acid can be a heterologous nucleic acid encoding the npr1(SAL) protein or a modified endogenous NPR1 gene.

The heterologous nucleic acid encoding the npr1(SAL) protein can encode an Arabidopsis npr1(SAL) protein or an ortholog of an Arabidopsis npr1(SAL) protein. In some embodiments, the Arabidopsis npr1(SAL) protein comprises the amino acid sequence of SEQ ID NO: 1. In some embodiments, the ortholog of the Arabidopsis npr1(SAL) comprises the amino acid sequence of any one of SEQ ID NOs: 5-22. In some embodiments, the heterologous nucleic acid comprises a sequence having at least 75%, at least 80%, at least 85%, at least 90%, at least 95%, or 100% identity to the nucleic acid sequence of SEQ ID NO:2, or an ortholog thereof, and encodes a npr1(SAL) protein. In some embodiments, the described genetically modified plants comprise a heterologous nucleic acid sequence encoding an ortholog of the Arabidopsis npr1(SAL) protein, wherein the ortholog is derived from a plant of the same species or cultivar as the genetically modified plant.

In some embodiments, the modified endogenous NPR1 gene comprises cysteine substitutions at positions 400 and 506, wherein the positions are relative to the AtNPR1 amino acid sequence (SEQ ID NO:3). In some embodiments, the modified endogenous NPR1 further comprises an alanine or leucine substitution at position 401 and/or an alanine or leucine substitution at position 402, wherein the positions are relative to the AtNPR1 amino acid sequence (SEQ ID NO:3) (e.g., as provided in SEQ ID NOs. 5-22). An endogenous NPR1 gene in a plant can be modified using CRISPR or other gene modification tools.

In some embodiments, the described genetically modified plants have improved or increased growth under stress conditions. The stress can comprise biotic stress or abiotic stress. In some embodiments, the described genetically modified plants have increased resistance to pathogen infection. In some embodiments, the described genetically modified plants have increased growth under stress conditions relative to a similar plant overexpressing NPR1.

Also described are methods of increasing growth of a plant during stress, the method comprising: expressing in the plant a nucleic acid encoding a npr1(SAL) protein. The stress can comprise biotic stress or abiotic stress. In some embodiments, expressing in the plant a nucleic acid encoding a npr1(SAL) protein comprises introducing a heterologous nucleic acid encoding the npr1 (SAL) protein into a plant cell. In some embodiments, expressing in the plant a nucleic acid encoding a npr1(SAL) protein comprises modifying an endogenous NPR1 gene in a plant cell to encode cysteines at positions 400 and 506, wherein the positions are relative to the Arabidopsis NPR1 protein (SEQ ID NO:3). In some embodiments, modifying the endogenous NPR1 gene in a plant cell further comprises modifying the NPR1 gene to encode an alanine or leucine at position 401 and/or 402, wherein the positions are relative to the Arabidopsis NPR1 protein (SEQ ID NO:3).

In some embodiments, expressing the nucleic acid encoding the npr1(SAL) protein in the plant increases resistance to pathogen infection. In some embodiments, the resulting plants have increased growth under stress conditions relative to similar plants overexpressing wild-type NPR1.

In some embodiments, expressing in the plant the nucleic acid encoding the npr1(SAL) protein comprises introducing into the plant cell's genome a heterologous nucleic acid encoding the npr1(SAL) protein. In some embodiments, expressing in the plant the nucleic acid encoding the npr1(SAL) protein comprises introducing a CRISPR system into a plant cell, wherein the CRISPR system introduces into the plant cell's genome a heterologous nucleic acid encoding the npr1(SAL) protein. In some embodiments, the heterologous nucleic acid encodes an Arabidopsis npr1(SAL) protein. In some embodiments, the heterologous nucleic acid encodes an ortholog of an Arabidopsis npr1(SAL) protein. In some embodiments, the heterologous nucleic acid encodes: SEQ ID NO: 1 or an ortholog thereof or any one of SEQ ID NOs: 5-22. n some embodiments, the heterologous nucleic acid comprises a nucleic acid having at least 75%, at least 80%, at least 85%, at least 90%, at least 95%, or 100%, identity to SEQ ID NO: 2, or an ortholog thereof, and encodes a npr1(SAL) protein. In some embodiments, the ortholog of the Arabidopsis npr1(SAL) protein is derived from a plant of the same species or cultivar as the plant.

In some embodiments, expressing in the plant the nucleic acid encoding the npr1(SAL) protein comprises modifying the endogenous NPR1 gene to encode cysteines at positions 400 and 506, wherein the positions are relative to the Arabidopsis NPR1 protein (SEQ ID NO:3). In some embodiments, modifying the endogenous NPR1 gene further comprises further comprises modifying the NPR1 gene to encode an alanine or leucine at position 401 and/or 402, wherein the positions are relative to the Arabidopsis NPR1 protein (SEQ ID NO:3)

In some embodiments, the methods comprise generating one or more regenerants following introducing the heterologous nucleic acid or the modifying the endogenous NPR1 gene into a plant or plant cell. In some embodiments, the methods further comprise genotyping one or more regenerants for the presence of the nucleic acid encoding the npr1 (SAL) protein and/or selecting one or more To plants containing the nucleic acid encoding the npr1 (SAL) protein.

Also described are nucleic acid encodings npr1(SAL) proteins. In some embodiments, the nucleic acid encoding the npr1(SAL) protein comprises a nucleic acid encoding an Arabidopsis npr1(SAL) protein or an ortholog of the Arabidopsis npr1(SAL) protein. In some embodiments, the nucleic acid encoding the npr1(SAL) protein comprises a sequence having at least 75%, at least 80%, at least 85%, at least 90%, at least 95%, or 100% identity to SEQ ID NO: 2 or an ortholog thereof. In some embodiments, the nucleic acid encoding the npr1 (SAL) protein comprises a sequence encoding any one of SEQ ID NOs: 5-22.

Also described are nucleic acid constructs for modifying a plant or plant cell, wherein the nucleic acid construct comprises a nucleic acid encoding a npr1 (SAL) protein; or a nucleic acid for modifying an endogenous NPR1 gene in the plant or plant cell to encode cysteines at positions 400 and 506, and optionally an alanine or leucine at position 401 and/or 402, wherein the positions are relative to the Arabidopsis NPR1 protein (SEQ ID NO:3).

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIG. 1 is an illustration of biomass accumulation in npr1 (SAL) mutant under BTH treatment in accordance with one embodiment of the present disclosure; and

FIG. 2 is an illustration of survival phenotype of npr1 (SAL) mutant under pathogen-triggered cell death in accordance with one embodiment of the present disclosure.

FIG. 3 Cryo-EM analysis of apo NPR1 and NPR1-SA. (Panel a) The NPR1 homodimer (residues 40-402). (Panel b) Topology of NPR1 BTB, BHB and ANKs. Cys, cysteine cluster. (Panel c) Alignment of NPR1 BHB with the 3-box motif of SPOP (Protein Data Bank (PDB): 3HTM). (Panel d) Stabilization of NPR1 ANKs through a His300/His334-mediated hydrogen-bond network. bb, backbone amides. (Panel e) Representative 2D classes of NPR1 cryo-EM images without SA (left) and with SA (right). The arrows indicate the folded SBD. Scale bar, 5 nm. (Panel f), 3D reconstruction of full-length NPR1 showing SBD-ANK docking in the presence of SA. (Panel g) Hydrophobic interactions between SBD and ANK3/4. Interfacial residues (stick model), Ca atoms of Gln400 and Arg506, and SA are shown. (Panels h and i) Induction of the PRI promoter 24 h after SA treatment (Panel h) and interaction with TGA3-mRFP (Panel i) tested for HA-tagged NPR1 (NPR1-HA), npr1(L346D) (L346D-HA), npr1(L393D) (L393D-HA) and npr1(1397D) (1397D-HA). (Panels j and k) Induction of the PRI promoter 30 h after SA treatment (Panel j) and the interactions between TGA3-mRFP and NPR1 variants (Panel k), tested for NPR1-HA or npr1(Q400C/E401L/R506C) (Q400C/E401L/R506C-HA). For (Panel h) and (Panel j), data are mean±s.d. n=3 independent biological replicates. P values were calculated using one-way analysis of variance (ANOVA) with Tukey's post test. The images in (Panel i) and (Panel k) are representatives of two biological replicates each.

FIG. 4. NPR1 contains a unique zinc finger and a redox sensor. (Panel a) NPR1 homodimer (residues 39-405) with the zinc finger detailed. The coordination of Zn2 (coral sphere) by side chains of Zn2-chelating residues (shown in the stick model and labelled in blue) is indicated by black dashed lines. The hydrogen-bond network between the zinc finger and ANK4 is indicated by dashed lines, with the pertinent side chain and backbone groups shown in the stick model. The side chain of Cys156 is shown in the stick model. (Panel b) Consensus (top) and alignment showing conserved zinc finger residues (bottom) in NPRIs from different species generated by Clustal Omega34. Conserved residues in the consensus sequence are bold, whereas variable residues are indicated by Xs. Proteins with and without Cys156 are separated with a double line. Listed plant species include: Arabidopsis thaliana (At), Brassica rapa (Br), Brassica juncea (Bj), Brassica napus (Bn), Raphanus sativus (Rs), Nicotiana tabacum (Nt), Populus trichocarpa (Pt), Solanum lycopersicum (SI), Vitis vinifera (Iv), Medicago truncatula (Mt), Hordeum vulgare (Hv), Oryza sativa (Os), Zea mays (Zm), and Glycine max ((im). (Panels c and d) Interaction between TGA3-mRFP and NPR1 variants (Panel c) and induction of PRI promoter 24 h after SA treatment (Panel d), tested for HA-tagged NPR1 (NPR1-HA), npr1(C150A) (C150A-HA), npr1(C150Y) (C150Y-HA), npr1(C155A) (C155A-HA), npr1(C155Y) (C155Y-HA), npr1(H157A) (H157A-HA), npr1(C160A) (C160A-HA) and npr1(A151P/D152R) (A151P/D152R-HA). For Panel d, data are mean±s.d. n=3 independent biological replicates. P values were calculated using one-way ANOVA and Tukey's post test. (Panel e) The NPR1 (ASBD) (Thr39-Asp410) tetramer in the protein crystal. (Panel f) In vitro oligomerization of NPR1 (residues Thr39-Lys262), WT or the C156A, C82A and C212V/C216A/C223L mutants, induced by hydrogen peroxide. The hash symbol (#) indicates impurity. The images in Panels c and f are representative of two and three biological replicates, respectively.

FIG. 5. The structure and function of the NPR1-TGA3 complexes. (Panel a) Cryo-EM density map and cartoon representation of the TGA32-NPR12-TGA32 complex. (Panel b) Cryo-EM density map of the NPR12-TGA32 assembly intermediate. The ANK region with weak density is indicated. (Panel c) The palmitate-containing TGA3 NID dimer. (Panel d) Palmitic acid recognition by the TGA3 NID in the 1.5-Å-resolution crystal structure of the TGA3 NID-palmitate complex. Polar interactions are denoted (dashed lines), and van der Waals contacts are shown (curved lines). (Panel e) The molecular interactions between the NPR1 ANKI (upper, horizontal helices) and the TGA3 NID (lower, vertical helices). Polar interactions are indicated (dashed lines). (Panels f and g) Interactions between TGA3-mRFP and the indicated NPR I variants (Panel f) and the induction of the PRI promoter 24 h after SA treatment (Panel g), tested for HA-tagged NPR1 (NPR1-HA), npr1 (L281D) (L281D-HA), npr1(L284D) (L284D-HA) and npr1(L281D/L284D) (L281D/L284D-HA). For Panel g, data are mean±s.d. n=3 independent biological replicates. P values shown were calculated using one-way ANOVA and Tukey's post test. The image in Panel f is representative of two biological replicates.

FIG. 6. The NPR1 dimer is required for SA-mediated immunity activation. (Panel a) Electrophoresis mobility shift assay for TGA3 in complex with NPR1 or npr1(dim) (dim). The PRI promoter region with (LS5/LS7) or without (LS5-/LS7-) two as-/elements was used as the probe. (Panel b) The NPR1 dimerization interface. ((Panel c) Self-interaction of NPR1 or npr1(dim) tested between GFP-and HA-fused proteins, with and without SA. (Panel d) Cell-free oligomerization of NPR1-GFP, npr1(dim)-GFP or npr1(C82A)-GFP (C82A-GFP). M, monomerized; O, oligomerized; T, total reduced proteins. (Panel e) Localization (left) and nucleocytoplasmic partitioning (right) of NPR1-GFP and npr1(dim)-GFP. Scale bar, 20 μm. (Panel f) The induction of the PRI promoter by NPR1-HA or npr1 (dim)-HA 24 h after SA treatment. (Panels g and h) Defense gene expression (Panel g) and growth of Pseudomonas syringae pv. maculicola (Psm) ES4326 (Panel h) in transgenic npr1 plants expressing NPR1-GFP, npr1(dim)-GFP lines 3 and 5, or C82A-GFP treated with 1 mM SA for 24 h. The box plot in (Panel e) shows the median (center line), and 25th and 75th percentiles (box limits), with the whiskers marking the minimum and maximum values. n=15 (NPR1-GFP) and n=16 (npr1(dim)-GFP) micrographs examined over 3 independent biological replicates. c.f.u., colony-forming units. Data are mean±s.d. of n=3 independent biological replicates (Panels f and g); and mean±95% confidence intervals of n=8 independent biological replicates (Panel h). P values shown were calculated using either two-tailed Student's t-tests (Panel e), or one-way ANOVA (Panels f and g) and two-way ANOVA (Panel h) followed by Tukey's post test. The experiments in Panels c-h were repeated at least twice with similar results. (Panel i) The enhanceosome model of dimeric NPR1 bridging two dimeric TGA transcription factors. SA-induced SBD-ANK docking creates a new interface to facilitate post-translational modifications and/or to facilitate the recruitment of transcriptional regulators (X) for the activation of defense genes. The images are representative of three (Panels a, c, and e) and four (Panel d) biological replicates.

FIG. 7. Biochemical characterization and cryo-EM reconstruction of apo NPR1. (Panel a) SDS-PAGE gel of purified NPR1. (Panel b) Size-exclusion chromatography shows that NPR1 elutes between molecular weight markers of 440 kDa and 158 kDa on a Superose 6 Increase 10/300 GL column. (Panel c) Crosslinking of NPR1 by BS3 at varying concentrations reveals a dominant dimer band. (Panel d) Flowchart of the reconstruction. Details are described in Methods. Circles highlight representative NPR1 particles on the micrograph with the enlarged view in the inset. (Panel e) The global Fourier Shell Correlation (FSC) curve. (Panel f) Local resolution map. (Panel g) Euler angle distribution of the particles. (Panel h) Representative regions of the EM density map. Images in Panels a and c are representative of 3 and 2 biological replicates, respectively.

FIG. 8. Biochemical characterization of the NPR1 BHB and SBD domains. (Panel a) Interaction of Myc-tagged CUL3A (Myc-CUL3) with free GST, or GST-fused WT NPR1 (GST-NPR1), or NPRIABHB deletion mutant (deletion of E200-L258; GST-NPR1ABHB) in E. coli. The proteins were co-expressed in E. coli and total lysate was used for pull-down with glutathione affinity resin. Images are representative of 2 biological replicates. (Panel b) Solution small angle X-ray scattering (SAXS) data for the Arabidopsis apo NPR1 SBD purified from insect cells at a concentration of 1 mg/mL. The SAXS scattering curve, the Kratky plot, and the Guinier analysis are shown in the top, middle, and bottom panels, respectively. The non-bell shape of the Kratky plot indicates the protein is unfolded and exhibits a random-coil behavior. The Guinier analysis yields a large radius of gyration (Rg) value of 47 Å for the NPR1 SBD (˜20 kDa) in comparison with folded lysozyme (14.3 kDa; Rg=˜16 Å) and Bovine serum albumin (66 kDa; Rg=˜30 Å), indicating a disordered conformation.

FIG. 9. NPR1 harbors a unique zinc finger. (Panel a) X-ray fluorescence scanning data revealed the presence of Zn²⁺ in NPR1 (ASBD) crystals. Scanning results for the NPR1 protein crystal and buffers are shown in the left and right panels, respectively. (Panel b) Sequence alignment of BTB domains. Conserved cysteine and histidine residues in a unique cysteine cluster preserved in NPR proteins (150, 155, 157, and 160) are indicated. Dots indicate residues participating in zinc coordination, and triangles denote residues mutated in npr1(dim). Listed plant species include: Arabidopsis thaliana (At), Brassica rapa (Br), Brassica juncea (Bj), Brassica napus (Bn), Raphanus sativus (Rs), Oryza sativa (Os), Nicotiana tabacum (Nt), Populus trichocarpa (Pt), Zea mays (Zm), Solanum lycopersicum (SI), Vitis vinifera (Vv), Hordeum vulgare (Hv), Medicago truncatula (Mt), and Glycine max (Gm). (NPR1-At/39-198 (SEQ ID NO:41), NPR1-Br/40-196 (SEQ ID NO:42), NPR1-Bj/40-196 (SEQ ID NO:43), NPR1-Bn/40-196 (SEQ ID NO:44), NPR1-Rs/41-197 (SEQ ID NO:45), NPR1-Os/27-198 (SEQ ID NO:47), NPR1-Nt/36-193 (SEQ ID NO:48), NPR1-Pt/32-190 (SEQ ID NO:49), NPR1-Zm/29-196 (SEQ ID NO:50), NPR1-SI/28-182 (SEQ ID NO:51), NPR1-Vv/34-188 (SEQ ID NO:52), NPR1-Hv/25-189 (SEQ ID NO:53), NPR1-Mt/39-193 (SEQ ID NO:54). and NPR1-Gm/26-186 (SEQ ID NO:55).

FIG. 10. Cryo-EM reconstruction of the NPR1-SA complex. Details of the flowchart are described in Methods. (Panel a) Flowchart of the reconstruction. (Panel b) Global Fourier Shell Correlation (FSC) curve. (Panel c) Euler angle distribution of the particles.

FIG. 11. Cryo-EM reconstruction of the NPR1-TGA3 complex. (Panel a) Flowchart of the reconstruction. (Panel b) Local resolution, global Fourier Shell Correlation (FSC) curve, Euler angle distribution of the particles, and representative regions of the EM density map of the TGA32-NPR12-TGA32 complex. (Panel c) Local resolution, global FSC curve, Euler angle distribution of the particles, and representative regions of the EM density map of the NPR12-TGA32 complex.

FIG. 12. Crystallographic and mass spectrometry characterization of the TGA3 NID-palmitate complex. (Panel a) Crystal structure of the TGA3 NID dimer. The two TGA3-NID molecules are shown. (Panel b) A zoomed-in view of the location of the palmitate. Polar interactions with the carboxylate group of the palmitates are indicated with dashed lines. Meshes in panels a and b represent 2mFo-DFc omit map of the palmitate plotted at the 1.0° C. level. (Panel c) Mass spectrometry analysis of the fatty acid extracted from the protein sample, verifying the fatty acid as the palmitic acid (C16: 0).

FIG. 13. The as-I elements in SA-induced gene promoters. (Panel a) Distribution of as-1 element in the promoters (3 kb) of SA-uninduced genes compared to the promoters (3 kb) of top 100 SA-induced genes. A statistically significant difference of as-1 element distribution was seen in the promoters of the top 100 SA-induced genes compared to the promoters of SA-uninduced genes (p-value <0.001). (Panel b) Promoter analysis for as-1 elements of the top 100 SA-induced genes after 8 h treatment. (Panel c) Frequency plot of distances between as-1 elements from the promoters of the top 100 SA-induced genes. (Panel d) Electrophoresis mobility shift assay of NPR1 and TGA3 using 6-fluorescein-labelled DNA spanning the LS5-to-LS7 region of the PRI promoter containing two as-/elements (LS5/LS7) or a single as-1 element in the LS5 region (LS5/LS7) or in the LS7 region (LS5/LS7). Panel d is a representative image of 3 biological replicates.

FIG. 14. Characterization of the npr1 (dim) mutant. (Panel a) Elution profiles of the WT NPR1, npr1(C82A), and npr1(dim) samples on a Superose 6 Increase 10/300 GL column. (Panel b) A low resolution cryo-EM map of npr1(dim) reveals the expected shape of a monomeric NPR1. The monomeric model of NPR1 has been fitted into the EM density and displayed in the cartoon representation.

FIG. 15. Sequence alignment corresponding to amino acids 353-509 of Arabidopsis NPR1 (Arabidopsis_NPR1/353-509 (SEQ ID NO: 23), Canola_NPR1/351-495 (Brassica rapa, Brassica juncea, Brassica napus; SEQ ID NO:24), Wheat_NPR1/349-502 (SEQ ID NO:25), Barley_NPR1/348-501 (SEQ ID NO:26), Rice_NPR1/357-512 (SEQ ID NO:27), Corn_NPR1/355-509 (SEQ ID NO:28), Sorghum_NPR1/355-509 (SEQ ID NO:29), Strawberry_NPR1/350-506 (SEQ ID NO:30), Soybean_NPR1/345-501 (SEQ ID NO:31), Navel_orange_NPR1/351-507 (SEQ ID NO:32), Mandarin_NPR1/351-507 (SEQ ID NO:33), Tomato_NPR1/341-496 (SEQ ID NO:34), Grape_NPR1/347-503 (SEQ ID NO:35). Additional sequences corresponding to amino acids 353-509 of Arabidopsis NPR 1 are provided in: Radish (SEQ ID NO:36), Tobacco (SEQ ID NO:37), Black cottonwood (SEQ ID NO:38), Melon (SEQ ID NO: 39), and Barrelclover (SEQ ID NO:40).

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### I. Definitions

Articles “a” and “an” are used herein to refer to one or to more than one (i.e., at least one) of the grammatical object of the article. By way of example, “an element” means at least one element and can include more than one element.

The term “about” or “approximately” indicates within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of 0 to 20%, 0 to 10%, 0 to 5%, or up to 1% of a given value. Where particular values are described in the application and claims, unless otherwise stated the term “about” means within an acceptable error range for the particular value should be assumed.

The use herein of the terms “comprises,” “comprising,” “contain,” “contains,” “containing,” “include,” “includes,” and “including” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “effective amount” refers to an amount sufficient to effect beneficial or desirable biological results.

The term “locus” refers to a position in the genome that corresponds to a measurable characteristic (e.g., a trait) or gene. A locus can be a genomic region or section of DNA (the locus) which correlates with a variation in a phenotype. A locus can comprise a single or multiple genes or other genetic information within a contiguous genomic region or linkage group.

A “heterologous” sequence is a sequence which is not normally present in a cell, genome, or gene in the genetic context in which the sequence is currently found. A heterologous sequence can be a sequence derived from the same gene (e.g., a different allele) and/or cell type, but introduced into the cell or a similar cell in a different context, such as on an expression vector or in a different chromosomal location or with a different promoter. A heterologous sequence can be a sequence derived from a different gene or species than a reference gene or species. A heterologous sequence can be from a homologous gene from a different species, from a different gene in the same species, or from a different gene from a different species. For example, a regulatory sequence may be heterologous in that it is linked to a different coding sequence relative to the native regulatory sequence.

“Orthologs” are genes and products thereof in different species that evolved from a common ancestral gene by speciation and retain the same or similar function. An ortholog is a gene that is related by vertical descent and is responsible for substantially the same or identical functions in different organisms. For example, Arabidopsis thaliana NPR1 and maize NPR1 can be considered orthologs. Genes may share sequence similarity of sufficient amount to indicate they are orthologs. Protein may share three-dimensional structure of sufficient amount to indicate the proteins and the genes encoding them are orthologs. Methods of identifying orthologs are known in the art.

A “homeolog” or “homeologous” genes refer to genes found in the same species that originated by a speciation event. Homeologs can evolve in polyploidy species from allopolyploidization, a whole-genome duplication via hybridization followed by genome doubling. For example, an octaploid plant may contain four homeologous genes that evolved from allopolyploidization, which are homologous to a single gene in the diploid plant.

The term “nucleic acid” refers to deoxyribonucleotides or ribonucleotides and polymers thereof (“polynucleotides”) in either single-or double-stranded form. Unless specifically limited, the term polynucleotide encompasses nucleic acids containing known analogues of natural nucleotides which have similar binding properties as the reference nucleic acid and are metabolized in a manner similar to naturally occurring nucleotides. Unless specifically limited, the term polynucleotide encompasses nucleic acids having one or more modified nucleotides. Modified nucleotides can modify binding properties or alter in vitro or in vivo stability. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985 J. Biol. Chem. 260:2605-2608; and Cassol et al., 1992; Rossolini et al., 1994, Mol. Cell. Probes 8:91-98). The term nucleic acid is used interchangeably with gene, cDNA, and mRNA encoded by a gene.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted, and other elements added without sacrificing the necessary expression.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a sequence comparison algorithms, or by manual alignment and visual inspection.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

A residue (e.g., amino acid) in an ortholog of AtNPR1 can be determined to correspond with a residue in AtNPR1 by optimally aligning the two sequences for maximum correspondence over the full sequence or over a specified comparison window. In aligning the sequences, either sequence (AtNPR1 or the ortholog of ANPR1) may contain amino acid additions (insertions) or deletions (gaps) relative to the other sequence. An amino acid residue of an ortholog of AtNPR1 corresponds to an amino acid residue at a particular position of AtNPR1 if amino acid residues are located at the same position when the AtNPR1 sequence and the AfNPR1 ortholog sequence are optimally aligned.

The term “plant” includes whole plants, plant organs (e.g., leaves, stems, flowers, roots, reproductive organs, embryos, and parts thereof, etc.), seedlings, seeds and plant cells, and progeny thereof. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, (e.g., octoploid, diploid, haploid, and hemizygous).

An “RNA-guided DNA endonuclease” is an enzyme (endonuclease) that uses RNA-DNA complementarity to identify target sites for sequence-specific double-stranded DNA (dsDNA) cleavage. An RNA-guided DNA endonuclease may be, but is not limited to, a zCas9 nuclease, a Cas9 nuclease, type II Cas nuclease, an nCas9 nuclease, a type V Cas nuclease, a Cas12a nuclease, a Cas12b nuclease, a Cas12c nuclease, a CasY nuclease, a CasX nuclease, a Cas12i nuclease, or an engineered RNA-guided DNA endonuclease.

A “guide RNA” (gRNA) comprises an RNA sequence (tracrRNA) bound by Cas and a spacer sequence (crRNA) that hybridizes to a target sequence and defines the genomic target to be modified. The tracrRNA and crRNA may be linked to form a “single chimeric guide RNA” (sgRNA).

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339:823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN [A/C/T] RRT, NGAN, NGCG, NGAG, NGNG, NGC, and NGA.

A “trans-activating CRISPR RNA” (tracrRNA) is an RNA species facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

A “DNA donor template” is a nucleic acid, such as a single stranded DNA, linear double strand DNA, plasmid DNA or AAV sequence that provides the homology necessary for precise repair of a double-strand break. A DNA donor template contains two regions of homology, one region of homology to either side of the double strand break. A DNA donor template can also contain a heterologous sequence between the two regions of homology. Each region of homology can be about 30 to about 100 nucleotides in length.

A “regenerant” is a plant produced from a plant tissue cell, such as a genetically modified plant tissue cell.

“Introgression” or “introgressing” of a locus means introduction of a locus from a donor plant comprising the locus of interest into a recipient plant by standard breeding techniques, wherein selection can be done phenotypically by means of observation of a plant characteristic including, but not limited to, characteristics such as the internodal length or plant height, or selection can be done with the use of markers through marker-assisted breeding, or combinations of these. The process of introgressing is often referred to as “backcrossing” when the process is repeated two or more times. In introgressing or backcrossing, the “donor” parent refers to the parental plant with the desired gene or locus to be introgressed. The “recipient” parent (used one or more times) or “recurrent” parent (used two or more times) refers to the parental plant into which the gene or locus is being introgressed. Selection is started in the F1 or any further generation from a cross between the recipient plant and the donor plant, suitably by using markers as identified herein. The skilled person is, however, familiar with creating and using new molecular markers that can identify or are linked to a locus of interest.

Unless otherwise defined, all technical terms used herein have the same meaning as commonly understood by one of ordinary skill in the art to which this disclosure belongs.

### II. NPR1 Variants

The Nonexpressor of Pathogenesis-Related Genes 1 (NPR1) gene is a positive regulator of the salicylic acid (SA)-mediated systemic acquired resistance (SAR) in plants. NPR1 is evolutionary conserved across a wide range of species, and overexpression of A/NPR1 protein enhances broad-spectrum disease resistance in numerous plants. NPR1 is recognized as a key regulator of defense across diverse plant species. NPR1 orthologs have been identified in numerous plant species, including, but not limited to, Brassica rapa (includes turnip Komatsuna, napa cabbage, bomdong, bok choy, and rapini, mustard seed/rapeseed), Brassica juncea (includes mustard, oilseed mustard), Brassica napus (rapeseed/canola), Oryza sativa (rice), Raphanus sativus (radish), Nicotiana tabacum (tobacco), Glycine max (soybean), Populus trichocarpa (black cottonwood), Cucumis melo (melon), Zea mays (corn/maize), Solanum lycopersicum (tomato), Vitis vinifera (grape), Hordeum vulgare (barley), Medicago truncatula (barrelclover), Sorghum bicolor (Sorghum), Saccharum species (sugar cane), Beta vulgaris (sugar beet), Panicum virgatum (switchgrass), Fragaria species (strawberry), and Citrus species (citrus; e.g., Citrus×sinensis (orange)).

The inventors have modeled a molecular mechanism of NPR1 activity in plant protection in which NPR1 promotes cell survival by targeting over-accumulated stress proteins for ubiquitination and degradation through formation of SA-induced NPR1 condensates (SINCs). SINCs are enriched with defense proteins, including a number of immune receptors, oxidative and DNA damage response proteins, and protein quality control machineries. Transition of NPR 1 into condensates is required for formation of the NPR1-Cullin 3 E3 ligase complex to ubiquitinate SINC-localized proteins and promote cell survival. NPR1 not only sequesters stress proteome in SINCs, but also induces transcription of a large number of defense genes, including SINC components, through its SA-dependent transcriptional coactivation function.

Structural analysis of NPR1 revealed a bird-shaped homodimer consisting of a central BTB domain, a BTB-and carboxy terminal Kelch helix bundle (BHB), four ankyrin repeats (ANK), and a disordered SA-binding domain (SBD). In the BTB domain, NPR1 harbors a unique zinc-finger motif important for interacting with ankyrin repeats and mediating NPR1 oligomerization. Cryo-EM analysis of NPR1 in complex with SA revealed that SA induces not only folding of the otherwise unstructured SBD domain, but also promotes its docking onto the ankyrin repeats.

A NPR1 protein includes AtNPR1 and orthologs of AtNPR1 from other plants (i.e., NPR1 proteins encoded by NPR1 genes). In some embodiments, AtNPR1 comprises SEQ ID NO: 3. NPR1 orthologs include, but are not limited to, Brassica rapa BrNPR1, Brassica juncea BjNPR1, Brassica napus BnNPR1, Raphanus sativus RsNPR1, Oryza sativa OsNPR1, Nicotiana tabacum NINPR1, Glycine max GmNPR1, Populus trichocarpa PINPR1, Cucumis melo CmNPR1, Zea mays ZmNPR1, Solanum lycopersicum SINPR1, Vitis vinifera WyNPR1, Hordeum vulgare HvNPR1, Medicago truncatula MINPR1, Sorghum bicolor SbNPR1, Musa acuminata MaNPR 1 and Musa balbisiana MbNPR1.

A NPR1 gene comprises a nucleotide sequence that encodes a NPR1 protein. A NPR1 gene includes AtNPR1 and orthologs of AtNPR1 form other plants. In some embodiments, ANPR1 comprises SEQ ID NO:4. NPR1 orthologs include, but are not limited to, Brassica rapa BrNPR1, Brassica juncea BjNPR1, Brassica napus BnNPR1, Raphanus sativus RsNPR1, Oryza sativa OsNPR1, Nicotiana tabacum NINPR1, Glycine max GmNPR1, Populus trichocarpa PINPR1, Cucumis melo CmNPR1, Zea mays ZmNPR1, Solanum lycopersicum SINPR1, Vitis vinifera VvNPR1, Hordeum vulgare HvNPR1, Medicago truncatula MINPR1, Sorghum bicolor SbNPR1, Avena sativa (oats), Triticum species (wheat), Secale cereale (rye), Rubus species (raspberries), Vaccinium species (blueberries, cranberries, etc.), Malus domestica (apples), Prunus species (peaches, plums, cherries, almonds, etc.), Gossypium species (cotton), Coffee species (coffee), Arachis hypogaea (peanut), Cannabisspecies (hemp), and palms (e.g., Cocos nucifera, Elaeis species, Nypa fruticans, Phoenix species, and Arenga pinnata).

Described are npr1 variants comprising cysteine substitutions at positions 400 and 506 relative to the Arabidopsis thaliana NPR1 protein (AtNPR1, SEQ ID NO:3), Q400C and R506C, respectively. The cysteine at position 400 is in an ANK domain, and the cysteine at position 506 is in the SBD domain. In some embodiments, the cysteines substituted at positions 400 and 506 (relative to SEQ ID NO:3) form a disulfide bond that crosslinks the SBD-ANK docking conformation. In some embodiments, the npr1 variants further comprise an Alanine or Leucine substitution at position 401 relative to ANPR1) protein (A1NPR1, SEQ ID NO:3), e.g., E401L. In some embodiments, the npr1 variants further comprise an Alanine or Leucine substitution at position 402 relative to AfNPR1), e.g., or 402A or 402L. The npr1(SAL) 401 (A or L) substitution or the 402 (A or L) substitution may compensate for the loss of the E401-R506 hydrogen bond and avoid burial of a charged residue at the protein interface. The resulting npr1 variants (400C/506C; 400C/401 (A or L/506C; 400C/402 (A or L)/506C), which can be referred to as npr1 (SAL) (for SBD-ANK Locked), significantly elevate induction of PRI gene expression in a transient reporter assay without changing the interaction with the TGA3 transcription factor. Activity of npr1 (SAL) indicates that the SBD-ANK-docking conformation is important for NPR1 activity in defense gene transcription.

In some embodiments, a Arnprl (Sal) variant comprises AtNPR1 having an Q400C substitution and a R506C substation. In some embodiments, the Atnprl (Sal) variant further comprises an E401A (SEQ ID NO:1) or E401L substitution.

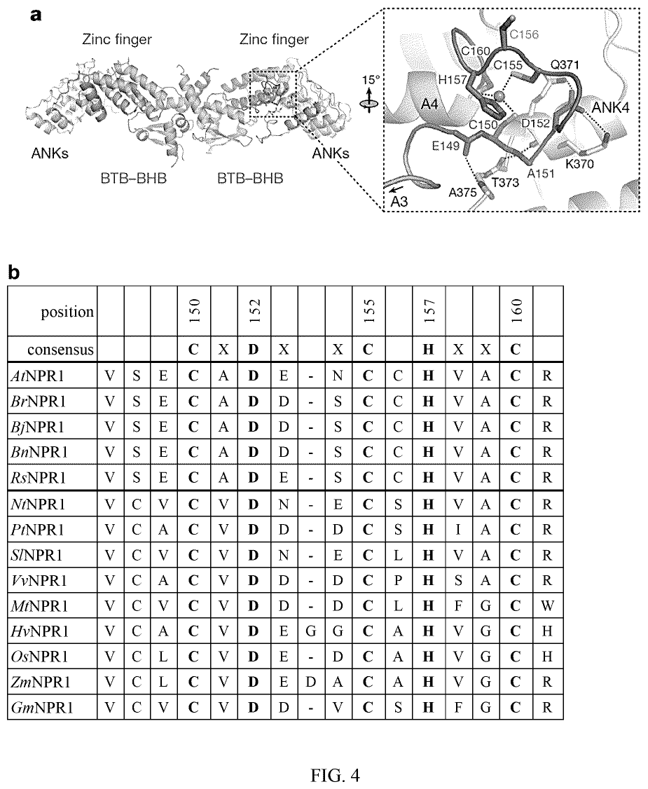

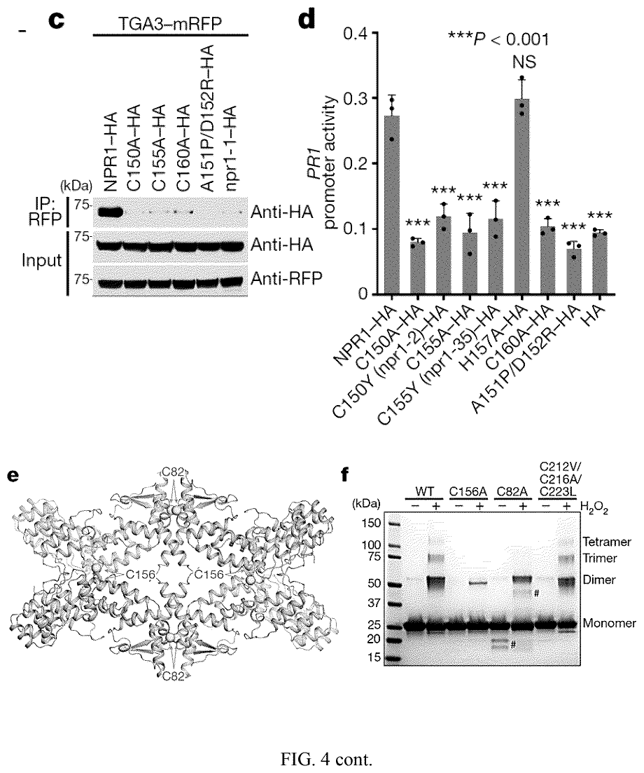

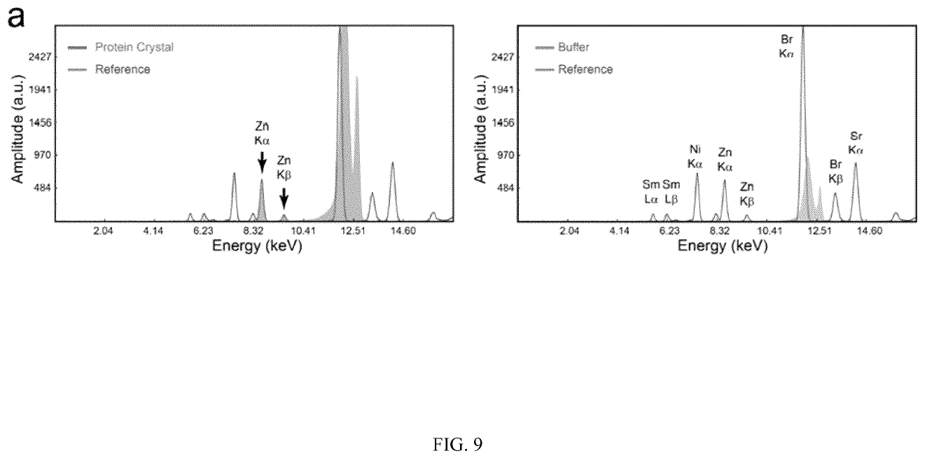

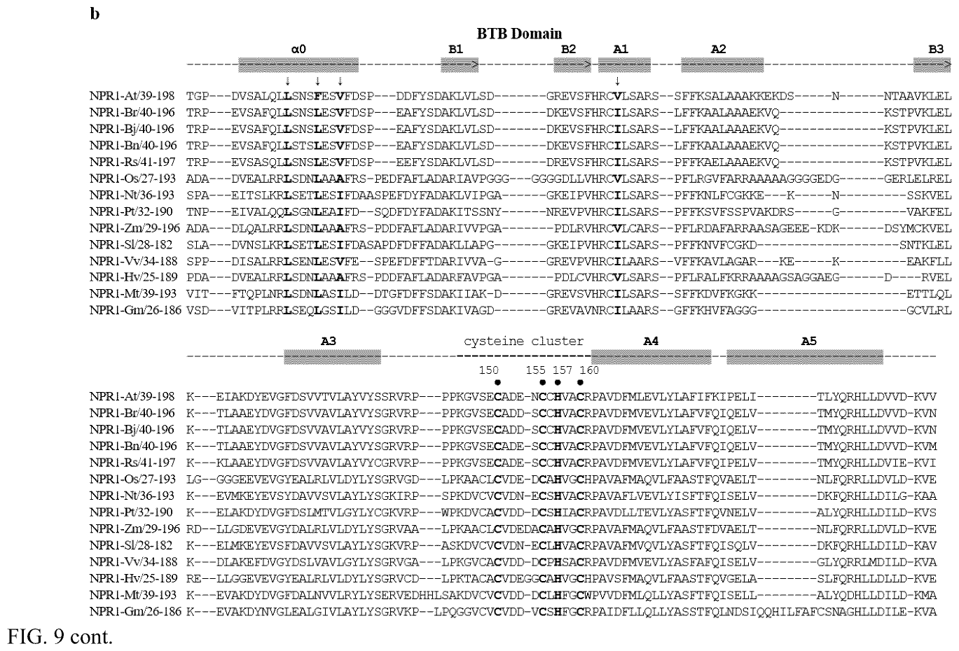

Similar substitutions can be made in orthologs of AtNPR1 from other plant species. NPR1 orthologs (and genes encoding the NPR1 orthologs) can be readily identified using methods and analyses available in the art for identifying orthologs in plants. Thus, a npr1 (SAL) variant includes Atnpr1(Sal) and orthologs of Amnpr1(SAL) from other plant species. In some embodiments, an Atnpr1(SAL) ortholog comprises an ortholog of ANPR 1 comprising cysteine substitutions at positions corresponding to positions 400 and 506 of AtNPR1 (SEQ ID NO:3). In some embodiments, an Atnpr1(SAL) ortholog further comprises an alanine or leucine substitution at a position corresponding to position 401 or 402 of AtNPR1 (SEQ ID NO:3)). In some embodiments, npr1(SAL) variants include, but are not limited to: Brassica rapa Brnpr1(SAL) (SEQ ID NO:5), Brassica juncea Bjnpr1(SAL) (SEQ ID NO:6), Brassica napus Bnnpr1(SAL) (SEQ ID NO:7), Raphanus sativus Rsnpr1(SAL) (SEQ ID NO:8), Oryza sativa Osnpr1(SAL) (SEQ ID NO:9), Nicotiana tabacum Ntnpr1(SAL) (SEQ ID NO:10), Glycine max Gmnpr1(SAL) (SEQ ID NO:11), Populus trichocarpa Pinpr1(SAL) (SEQ ID NO:12), Cucumis melo (mnpr1(SAL) (SEQ ID NO:13), Zea mays Zmnpr1(SAL) (SEQ ID NO:14), Solanum lycopersicum S/npr1(SAL) (SEQ ID NO:15), Vitis vinifera Vvnpr1(SAL) (SEQ ID NO: 16), Hordeum vulgare Hynpr1(SAL) (SEQ ID NO:17), Medicago truncatula Minpr1(SAL) (SEQ ID NO:18), Sorghum bicolor Sbnpr1(SAL) (SEQ ID NO:19), Triticum species Trnpr1(SAL) (SEQ ID NO:20), Citrus×sinensis Csnpr1(SAL) (SEQ ID NO:21), Fragaria species Fynpr1(SAL) (SEQ ID NO: 22). Alignment of the BTB domains and zinc finger regions of various NPR 1 orthologs is shown in FIG. 4 (Panel b) and FIG. 9 (Panel b). In some embodiments, any of the above npr1(SAL) can further contain an Alanine or Leucine at a position corresponding to position 401 of AtNPR1 and/or, and Alanine or Leucine substitution at a position corresponding to the position 402 position of A/NPR1.

In some embodiments, a npr1(SAL) variant comprises a protein having an amino acid sequence at least 80%, at least 85%, at least 90%, at least 91%, at least 92%, at least 93%, at least 94%, at least 95%, at least 96%, at least 97%, at least 98%, or at least 99% identity to the amino acid sequence of Amnpr1(SAL) (SEQ ID NO:1) or a protein having at least 90%, at least 91%, at least 92%, at least 93%, at least 94%, at least 95%, at least 96%, at least 97%, at least 98%, or at least 99% identity to an ortholog of Atnprl (SAL), wherein the npr1 (SAL) protein comprises a cysteine at positions corresponding to positions 400 and 506 of AtNPR1 (SEQ ID NO:3) and optionally an alanine or leucine substitution at a position corresponding to position 401 and/or 402 of AtNPR1 (SEQ ID NO:3), and wherein the npr1 (SAL) variant induces expression of PRI gene expression in a transient in response to salicylic acid (SA). In some embodiments, the npr1(SAL) variant interacts with the TGA3 transcription factor.

In some embodiments, a npr1(SAL) variant comprises a protein having an amino acid sequence at least 80%, at least 85%, at least 90%, at least 91%, at least 92%, at least 93%, at least 94%, at least 95%, at least 96%, at least 97%, at least 98%, or at least 99% identity to the amino acid sequence of any one of SEQ ID NO:5-22, wherein the npr1(SAL) protein comprises a cysteine at positions corresponding to positions 400 and 506 of AtNPR1 (SEQ ID NO: 3) and optionally an alanine or leucine substitution at a position corresponding to position 401 and/or 402 of AtNPR1 (SEQ ID NO:3), and wherein the npr1(SAL) variant induces expression of PRI gene expression. In some embodiments, the npr1(SAL) variant interacts with the TGA3 transcription factor.

Described are nucleic acid sequences, expression vectors, or CRISPR constructs encoding a npr1(SAL) variant or a portion thereof. A npr1(SAL) gene comprises a nucleic acid encoding a npr1(SAL) protein. In some embodiments, a npr1(SAL) gene comprises SEQ ID NO: 2 (Atnpr1(SAL)). In some embodiments, a npr1(SAL) gene comprises a nucleotide sequence having at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO:2, wherein the nucleic acid sequence encodes an Atnpr1(SAL protein). In some embodiments, a npr1(SAL) gene encodes an ortholog of Atnpr1(SAL). In some embodiments, a npr1(SAL) gene comprises a nucleotide sequence encoding any one of SEQ ID NO:5-22 or a protein having at least at least 80%, at least 85%, at least 90%, at least 91%, at least 92%, at least 93%, at least 94%, at least 95%, at least 96%, at least 97%, at least 98%, or at least 99% identity to the amino acid sequence of any one of SEQ ID NO:5-22, wherein the npr1(SAL) protein comprises a cysteine at positions corresponding to positions 400 and 506 of AtNPR1(SEQ ID NO:3) and optionally an alanine or leucine substitution at a position corresponding to position 401 or 402 of AtNPR1(SEQ ID NO: 3), and wherein the npr1(SAL) variant induces expression of PRI gene expression in response to SA.

Described are compositions for genetically modifying a plant to express a npr1(SAL) variant. Genetically modifying a plant to express a npr1(SAL) variant comprises expressing a heterologous nucleic acid encoding a npr1 (SAL) gene in the plant or modifying an endogenous NPR1 gene in the plant to encode a npr1 (SAL) variant. Described are compositions for modifying a NPR1 locus to encode a npr1(SAL) variant.

Also described are methods of using the compositions to produce plants having increased resistance to stress. In some embodiments, plants expressing a npr1 (SAL) variant have improved or increased growth or biomass production under stress conditions relative to genetically similar plants that do not express the npr1 (SAL) variant, when grown under the same conditions. In some embodiments, plants expressing a npr1(SAL) variant have increased stress resistance relative to genetically similar plants that do not express the npr1(SAL) variant, when grown under the same conditions. In some embodiments, plants expressing a npr1(SAL) variant have improved or increased growth or biomass production and increased stress resistance relative to genetically similar plants that do not express the npr l (SAL) variant, when grown under similar stress conditions. In some embodiments, plants expressing a npr1(SAL) variant have improved or increased growth or biomass production under stress conditions relative to genetically similar plant that do not express the npr1(SAL) variant, when grown under the same conditions. In some embodiments, expression of a npr1(SAL) variant in a plant results in improved growth under conditions of immunity activation compared to a genetically similar plant that does not express npr1(SAL), when grown under similar conditions. The stress resistance can be biotic stress and/or abiotic stress. Biotic stress includes, but is not limited to, bacterial or viral infection. Abiotic stress includes, but is not limited to, heat and drought.

Described are methods of improving plant growth under stress conditions comprising expressing in the plant a npr1 (SAL) gene. In some embodiments, the methods comprise expressing a heterologous npr l (SAL) gene in the plant. In some embodiments, the methods comprise modifying an endogenous NPR1 gene to generate a npr 1 (SAL) gene.

In some embodiments, plants expressing a npr1(SAL) variant have increased biomass production under stress conditions relative to genetically similar plants overexpressing wild-type NPR1, when grown under stress conditions. The stress resistance can be biotic stress and/or abiotic stress. Biotic stress includes, but is not limited to, bacterial or viral infection. Abiotic stress includes, but is not limited to, heat and drought.

In some embodiments, plants expressing a npr1(SAL) variant have increased survival under pathogen effector-triggered (ETI) cell death relative to genetically similar plants that do not express the npr1(SAL) variant, when grown under the same conditions. In some embodiments, plants expressing a npr1(SAL) variant have increase cell survival against pathogen-induced cell death relative to genetically similar plants that do not express the npr1(SAL) variant, when grown under the same conditions.

Increasing resistance to stress often has an indirect relationship with plant growth. Increasing stress resistance often results in decreased plant growth. For example, overexpression of wild-type NPR1 in plants increases disease resistance, but results in reduced growth under stress conditions. The described nucleic acids and methods can be used to improve plant crop yield while maintaining or increasing resistance to stress. In some embodiments, plants expressing a npr I (SAL) gene have at least 5%, at least 10%, at least 15%, at least 20%, at least 25%, at least 30%, at least 40%, at least 50%, at least 60%, at least 70%, at least 80%, at least 90%, or at least 95%, at least 100% increased biomass production under stress conditions relative to genetically similar plants that do not express a npr1(SAL) gene and overexpress wild-type NPR1. In some embodiments, stress-induced loss of crop yield (e.g., plant biomass) in plants expressing a npr1(SAL) gene may be reduced by at least 5%, at least 10%, at least 15%, at least 20%, at least 25%, at least 30%, at least 40%, at least 50%, at least 60%, at least 70%, at least 80%, at least 90%, or at least 95%, or up to 100% compared to genetically similar plants that do not express a npr (SAL) gene and overexpress wild-type NPR1.

### III. Genetically Modified Plants

In some embodiments, genetically modified plants expressing a npr1(SAL) gene are described. The plant can, but is not limited to, a crop plant. Using the described nucleic acids encoding a npr1(SAL) variants or portions thereof, genetically modified plants that express a npr1(SAL) variant can be made. The genetically modified plants can be made using methods available in the art for generating genetically modified plants.

In some embodiments, the genetically modified plants contain a nucleic acid sequence encoding a heterologous npr1(SAL) gene. In some embodiments, the heterologous npr1(SAL) gene is introduced into a plant to improve or increase plant growth under conditions of immunity activation. In some embodiments, the heterologous npr1(SAL) gene is introduced into a plant to improve or increase plant growth under conditions of biotic or abiotic stress. In some embodiments, the heterologous npr1(SAL) gene is introduced into a plant to increase stress resistance without inhibiting growth.

In some embodiments, an endogenous NPR1 gene or locus in a plant is modified to encode a npr1(SAL) protein (i.e., an endogenous NPR1 gene is modified to contain a Q400C substitution and a R400C substitution, and optionally a 410 or 402 alanine or leucine substitution, wherein the positions correspond to the positions in the AtNPR1 (SEQ ID NO:3)). In some embodiments, the endogenous NPR1 gene or locus in a plant is modified to encode a npr1(SAL) protein to improve or increase plant growth under conditions of immunity activation. In some embodiments, the endogenous NPR1 gene or locus in a plant is modified to encode a npr1(SAL) protein improve or increase plant growth under conditions of biotic or abiotic stress. In some embodiments, the endogenous NPR1 gene or locus in a plant is modified to encode a npr1(SAL) protein increase stress resistance without inhibiting growth.

Various methods for introducing a transgene expression vector constructs of the invention into a plant or plant cell are well known to those skilled in the art, and any method capable of transforming the target plant or plant cell may be utilized.

Nucleic acids may be introduced into a plant, plant cell or cells using a number of methods known in the art, including but not limited to electroporation, DNA bombardment or biolistic approaches, microinjection, via the use of various DNA-based vectors such as Agrobacterium tumefaciens and Agrobacterium rhizogenes vectors, and CRISPR or CRISPR/Cas9. Once a plant cell has been successfully transformed, it may be cultivated to regenerate a transgenic plant (regenerant). T₀ transgenic plants may be used to generate subsequent generations (e.g., T₁, T₂, etc.) by selfing of primary or secondary transformants, or by sexual crossing of primary or secondary transformants with other plants (transformed or untransformed).

In some embodiments, Agrobacterium tumefaciens is used to deliver an expression vector or CRISPR system nucleic acids to a plant. Agrobacterium-mediated transformation of a large number of plants are extensively described in the literature (see, for example, Agrobacterium Protocols, Wan, ed., Humana Press, 2^{nd} edition, 2006). Various methods for introducing DNA into Agrobacteria are known, including electroporation, freeze/thaw methods, and triparental mating. In some embodiments, a pMON316-based vector is used in the leaf disc transformation system of Horsch et al. Other commonly used transformation methods include, but are not limited to, microprojectile bombardment, biolistic transformation, and protoplast transformation of naked DNA by calcium, polyethylene glycol (PEG) or electroporation (Paszkowski et al., 1984, EMBO J. 3:2727-2722; Potrykus et al., 1985, Mol. Gen. Genet. 199:169-177; Fromm et al., 1985, Proc. Nat. Acad. Sci. USA 82:5824-5828; Shimamoto et al., 1989, Nature, 338:274-276.)

In some embodiments, unaided homologous recombination, recombinase-based insertion, and DNA repair-based insertion targeted genetic modification (see, e.g., Dong et al., 2021, PNAS. 118 (22) e2004834117) can be used to insert (knock in) a heterologous npr 1 (SAL) gene into a plant, replace an endogenous NPR1 allele with a npr1(SAL) gene, or modify an endogenous NPR1 gene to express a npr1(SAL) protein. Recombinase-based insertion can comprise systems and constructs involving site-specific recombinases. Such systems include, but not limited to, Cre: loxP systems, Flp: FRT systems, Dre: rox systems, VCre: loxV systems, Gin: gix systems, Bxb1: attP attB systems, phiC31: attP attB systems. DNA repair-based insertion methods rely upon the activity of a nuclease agent. The nuclease agent can be, but is not limited to, a Transcription Activator-Like Effector Nuclease (TALEN), a zinc-finger nuclease (ZFN), a meganuclease, a restriction endonuclease (Type I, Type II, Type III, or Type IV endonucleases), or a CRISPR/Cas system.

A CRISPR system comprises an RNA-guided DNA endonuclease enzyme and a CRISPR RNA. In some embodiments, a CRISPR RNA is part of a guide RNA. In some embodiments, the RNA-guided DNA endonuclease enzyme is a Cas9 protein. In some embodiments, a CRISPR system comprises one or more nucleic acids encoding an RNA-guided DNA endonuclease enzyme (such as, but not limited to a Cas9 protein) and a guide RNA. A guide RNA can comprise a CRISPR RNA (crRNA) and a trans-activating CRISPR RNA (tracrRNA), either as separate molecules or a single chimeric guide RNA (sgRNA). The guide RNA contains a guide sequence having complementarity to a sequence in the target gene genomic region. Suitable crRNAs or gRNAs and nucleic acids encoding crRNAs or gRNAs can be made using methods and tools available in the art to design and manufacture crRNAs or gRNAs. The Cas protein can be introduced into the plant in the form of a protein or a nucleic acid (DNA or RNA) encoding the Cas protein (e.g., operably linked to a promoter expressible in the plant). The guide RNA can be introduced into the plant in the form of RNA or a DNA encoding the guide RNA (e.g., operably linked to a promoter expressible in the plant). In some embodiments, the CRISPR system further includes a DNA donor template. In some embodiments, the CRISPR system can be delivered to a plant or plant cell via a bacterium. The bacterium can be, but is not limited to, Agrobacterium tumefaciens.

In some embodiments, the CRISPR system is designed to knock in a heterologous npr1(SAL) gene into a target locus in the plant suitable for expression of a heterologous npr 1 (SAL) gene. The target locus can be an endogenous NPR1 locus or a safe harbor locus. In some embodiments, the CRISPR system is designed to modify an endogenous NPR1 locus.

The CRISPR/Cas system can be, but is not limited to, a CRISPR class 1 system, a CRISPR class 2 system, a CRISPR/Cas system, a CRISPR/Cas9 system, a CRISPR/zCas9 system or a CRISPR/Cas3 system.

Suitable guide sequences for use in a CRISPR system include 17-20 nucleotide sequences along the target locus that are unique compared to the rest of the genome and immediately adjacent (5′) to a protospacer-adjacent motif (PAM) site. For the RNA-guided DNA endonuclease enzyme zCas9, a PAM site is NGG. Thus, any unique 17-20 nucleotide sequence immediately 5′ of a 5′-NGG-3′ in the target locus can be used in forming a gRNA. In some embodiments, the guide sequence is 100% complementary to a target sequence present in the target locus. In some embodiments, the guide sequence is at least 90% or at least 95% complementary to the target sequence. In some embodiments, the guide sequence contains 0, 1, or 2 mismatches when hybridized to the target sequence. In some embodiments, a mismatch, if present, is located distal to the PAM, in the 5′ end of the guide sequence.

The DNA donor template contains sequence to be inserted into the genome of the plant. In some embodiments, the DNA donor template to be used with a CRISPR system comprises sequences for a heterologous npr 1 (SAP) gene, coding sequence, or fragment thereof. The donor template can further comprise one or more regulator sequence operatively linked to the coding sequence that drive expression of the coding sequence in the plant cell.

In some embodiments, the DNA donor template to be used with a CRISPR system comprises sequence for modifying an endogenous NPR1 gene. CRISPR constructs (nucleic acids) for modifying an endogenous NPR1 gene can be designed and manufactured using methods available in the art for genetically modifying a genomic locus in plants.

DNA donor templates further comprise 5′ and 3′ homology regions located 5′ and 3′ to the sequence to be inserted into the genome. The homology regions comprise about 30 to about 100 nucleotides that are complementary to a corresponding number of nucleotides in the genome on either side of the double strand break created by the CRISPR nuclease.

The DNA donor template can be provided a single strand DNA, double strand DNA, plasmid DNA, or adeno-associated vector DNA.

It is understood that RNA equivalents of any listed DNA sequences, substituting uracils (U) for thymines (T), may be used. An “RNA equivalent” is an RNA molecule having essentially the same complementary base pair hybridization properties as the listed DNA sequence.

CRISPR modification of a target locus is not limited to the CRISPR/zCas9 system. Other CRISPR systems using different nucleases and having different PAM sequence requirements are known in the art. PAM sequences vary by the species of RNA-guided DNA endonuclease. For example, Class 2 CRISPR-Cas type II endonuclease derived from S. pyogenes utilizes an NGG PAM sequence located on the immediate 3′ end of the guide sequence. Other PAM sequences include, but are not limited to, NNNNGATT (Neisseria meningitidis), NNAGAA (Streptococcus thermophilus), and NAAAAC (Treponema denticola). Guide sequences for CRISPR systems having nucleases with different PAM sequence requirements are identified as described above for zCas9, substituting the different PAM sequences.

Two or more guide RNAs can used with the same RNA-guided DNA endonuclease (e.g., Cas nuclease) or different RNA-guided DNA endonucleases.

Any of the above-described guide RNAs can be provided as an RNA or a DNA encoding the RNA.

In some embodiments, a CRISPR system comprises one or more guide RNAs and a nucleic acid encoding an RNA-guided DNA endonuclease. In some embodiments, a CRISPR system comprises one or more guide RNAs, a nucleic acid encoding an RNA-guided DNA endonuclease, and a DNA donor template.

In some embodiments, a CRISPR system comprises one or more guide RNAs and a one or more nucleic acids encoding two or more different RNA-guided DNA endonucleases. In some embodiments, a CRISPR system comprises one or more guide RNAs, one or more nucleic acids encoding two or more different RNA-guided DNA endonucleases, and at least one DNA donor template.

In some embodiments, a CRISPR system comprises a guide RNA and an RNA-guided DNA endonuclease in a complex. In some embodiments, a CRISPR system comprises a guide two or more RNAs each in a complex with an RNA-guided DNA endonuclease. In some embodiments, a CRISPR system comprises a guide RNA and an RNA-guided DNA endonuclease in a complex and a DNA donor template.

CRISPR constructs and systems comprising Cas proteins and guide RNAs for directed modification at the NPR1 gene are described. In some embodiments, the CRISPR constructs and systems can be used to modify an endogenous NPR1 gene to encode cysteine substitutions at positions corresponding to positions 400 and 506 of AtNPR1 (SEQ ID NO:3). In the embodiments, the CRISPR constructs and systems can be used to modify an endogenous NPR1 gene to contain encode cysteine substitutions at positions corresponding to positions 400 and 506 of AtNPR1 (SEQ ID NO:3) and an alanine or leucine substitution at a position corresponding to positions 401 or 402 of AINPR1. In some embodiments, CRISPR constructs and systems can be used to insert (knock in) a heterologous sequence encoding a npr1(SAL) gene into the genome of a plant. The heterologous sequence encoding a npr1(SAL) gene can be inserted into an endogenous NPR1 locus or a different locus, such as a safe harbor site, suitable for expression of the npr1(SAL) gene.

In some embodiments, the CRISPR constructs and systems are used to generate genetically modified plant cells carrying a npr1(SAL) gene. The npr1(SAL) gene can be a modified endogenous NPR 1 gene or a heterologous npr1(SAL) gene. These plant cells can then be used to produce regenerant progeny transgenic plants that that express the npr1(SAL) gene.

The described methods can be used to modify a plant having one or more traits of interest. Such traits can be, but are not limited to, genes for herbicide tolerance, increased yield, insect control, other fungal disease resistance, virus resistance, bacterial disease resistance, germination and/or seedling growth control, enhanced animal and/or human nutrition, improved processing traits, or improved flavor, among others.

### IV. Listing of Embodiments

1. A genetically modified plant, wherein the genetically modified plant comprises a nucleic acid encoding a npr1(SAL) protein.
2. The genetically modified plant of embodiment 1, wherein the nucleic acid comprises a heterologous nucleic acid encoding the npr1(SAL) protein.
3. The genetically modified plant of embodiment 2, wherein the heterologous nucleic acid encodes an Arabidopsis npr1(SAL) protein.
4. The genetically modified plant of embodiment 2, wherein the heterologous nucleic acid encodes an ortholog of an Arabidopsis npr1(SAL) protein.
5. The genetically modified plant of embodiment 3 or 4, wherein the heterologous nucleic acid:
   (a) encodes SEQ ID NO: 1 or an ortholog thereof;
   (b) encodes any one of SEQ ID NOs: 5-22;
   (c) comprises a nucleic acid sequence having at least 75% identity to SEQ ID NO: 2, or an ortholog thereof, and encodes a npr1(SAL) protein; or
   (d) comprises SEQ ID NO:2 or an ortholog thereof.
6. The genetically modified plant of embodiment 4, wherein the ortholog of the Arabidopsis npr1(SAL) protein is derived from a plant of the same species or cultivar as the genetically modified plant.
7. The genetically modified plant of embodiment 1, wherein the nucleic acid comprises a modified endogenous NPR1 gene, wherein the modified endogenous NPR1 gene is modified to contain a 400C substitution, a 506C substitution, and optionally a 401A, 401L, 402A, or 402L substitution, wherein the positions are relative to the ANPRI amino acid sequence (SEQ ID NO:3).
8. The genetically modified plant of embodiment 7, wherein the endogenous NPR1 gene is modified using CRISPR.
9. The genetically modified plant of any one of embodiments 1-8, wherein the genetically modified plant has increased growth under stress conditions.
10. The genetically modified plant of embodiment 9, wherein the stress comprises biotic stress or abiotic stress.
11. The genetically modified plant of embodiment 10, wherein the genetically modified plant has increased resistance to pathogen infection.
12. The genetically modified plant of any one of embodiments 1-11, wherein the genetically modified plant has increased growth under stress conditions relative to a similar plant overexpressing NPR1.
13. A method of increasing growth of a plant during stress, the method comprising: expressing in the plant a nucleic acid encoding a npr1(SAL) protein.
14. The method of embodiment 13, wherein expressing in the plant the nucleic acid encoding the npr1(SAL) protein comprises:
   (a) introducing a heterologous nucleic acid encoding the npr1(SAL) protein into a plant cell; or
   (b) modifying an endogenous NPR1 gene in a plant cell to encode cysteines at positions 400 and 506, and optionally an alanine or leucine at position 401 and/or 402, wherein the positions are relative to the Arabidopsis NPR1 protein (SEQ ID NO:3).
15. The method of embodiment 13, wherein expressing in the plant the nucleic acid encoding the npr1(SAL) protein comprises introducing a CRISPR system into a plant cell, wherein the CRISPR system:
   (a) introduces into the plant cell's genome a heterologous nucleic acid encoding the npr1(SAL) protein; or
   (b) modifies the endogenous NPR1 gene to encode cysteines at positions 400 and 506, and optionally an alanine or leucine at position 401 and/or 402, wherein the positions are relative to the Arabidopsis NPR1 protein (SEQ ID NO:3).
16. The method of embodiment 14 or 15, wherein the heterologous nucleic acid encodes an Arabidopsis npr1(SAL) protein.
17. The method of embodiment 14 or 15, wherein the heterologous nucleic acid encodes an ortholog of an Arabidopsis npr1(SAL) protein.
18. The method of embodiment 16 or 17, wherein the heterologous nucleic acid:
   (a) encodes SEQ ID NO: 1 or an ortholog thereof.
   (b) encodes any one of SEQ ID NOs: 5-22;
   (c) comprises a nucleic acid having at least 75% identity to SEQ ID NO:2, or an ortholog thereof, and encodes a npr1(SAL) protein; or
   (d) comprises SEQ ID NO:2 or an ortholog thereof.
19. The method of embodiment 17, wherein the ortholog of the Arabidopsis npr1(SAL) protein is derived from a plant of the same species or cultivar as the plant.
20. The method of any one of embodiments 13-18, wherein the stress comprises biotic stress or abiotic stress.
21. The method of embodiment 20, wherein the plant has increased resistance to pathogen infection.
22. The method of any one of embodiments 13-21, wherein the plant has increased growth relative to a similar plant overexpressing NPR1.
23. The method of any one of embodiments 14-22, wherein the method further comprises generating one or more regenerants following the introducing the heterologous nucleic acid or the modifying the endogenous NPR1 gene.
24. The method of embodiment 23, wherein the method further comprises genotyping one or more regenerants for the presence of the nucleic acid encoding the npr1(SAL) protein.
25. The method of embodiment 23 or 24, wherein the method further comprises selecting one or more To plants containing the nucleic acid encoding the npr1(SAL) protein.
26. A nucleic acid encoding a npr1(SAL) protein.
27 The nucleic acid of embodiment 26, wherein the nucleic acid encodes an Arabidopsis npr1(SAL) protein or an ortholog of the Arabidopsis npr1(SAL) protein.
28. The nucleic acid of embodiment 27, wherein the nucleic acid:
   (a) a sequence having at least 75% identity to SEQ ID NO:2 or an ortholog thereof;
   (b) SEQ ID NO: 2 or an ortholog thereof; or
   (c) a sequence encoding any one of SEQ ID NOs: 5-22.
29. A nucleic acid construct for modifying a plant or plant cell, wherein the nucleic acid construct comprises:
   (a) nucleic acid encoding a npr1(SAL) protein; or
   (b) a nucleic acid for modifying an endogenous NPR1 gene in the plant or plant cell to encode cysteines at positions 400 and 506, and optionally an alanine or leucine at position 401 and/or 402, wherein the positions are relative to the Arabidopsis NPR1 protein (SEQ ID NO:3).

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

All references cited herein are fully incorporated by reference, unless explicitly indicated otherwise. The present disclosure shall control in the event there are any disparities between any definitions and/or description found in the cited references.

### EXAMPLES

### Example 1. Expression of npr1(SAL) Increase Plant Growth in Stress Conditions

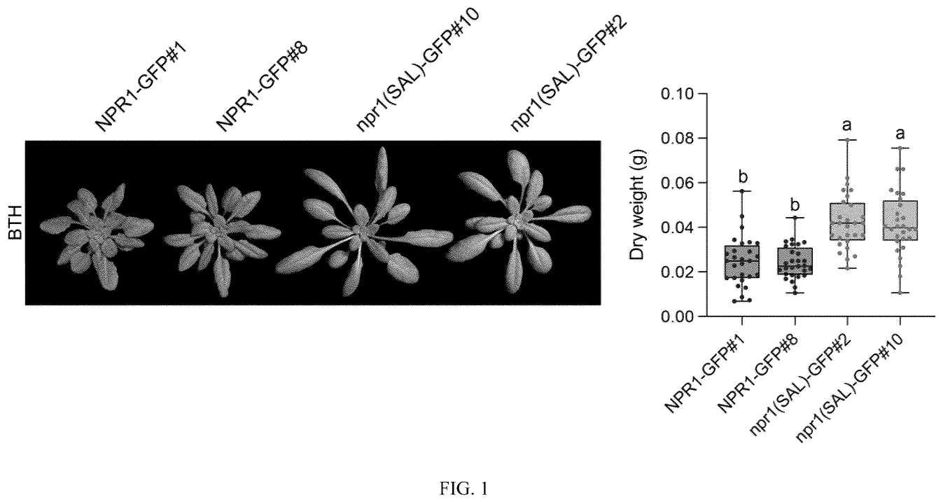

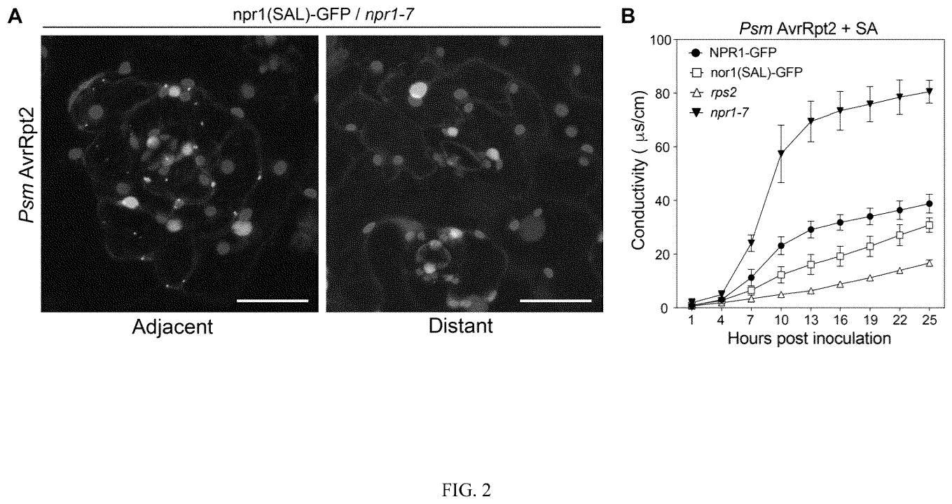

An important aspect of successful immunity is the ability to correctly manage defense-to-growth trade-offs. Repeated application of SA or its analog BTH has been traditionally used to induce growth retardation in Arabidopsis as a result of activation of NPR1-dependent immunity. To test the role of the npr1(SAL) variant in the SA-induced growth retardation, the npr1(SAL)-GFP fusion was stably expressed in transgenic plants and plant biomass after BTH treatment was measured (FIG. 1). It was found that the two independent transgenic lines of npr1(SAL)-GFP plants accumulated more biomass compared to the transgenic plants expressing WT NPR1-GFP (FIG. 1). The results show that the activity of the npr1(SAL) variants allow plants to grow better under immunity activation and resistance compared to the plants expressing the wild-type NPR1 protein.

FIG. 1 illustrates biomass accumulation in npr1(SAL)-expressing plants under BTH treatment. Two-week-old NPR1-GFPInpr1-7 (lines #1 and #8) and npr1 (SAL)-GFP/npr1-7 (lines #2 and #10) were sprayed with 350 μM BTH 4 times over a period of two weeks. The plants at the five-week-old age (left) were harvested and the dry weight of the entire rossette was measured (right). The values are plotted in a box-plot. Letters indicate statistically significant differences determined by one-way ANOVA followed by Tukey's correction for multiple comparisons at P<0.05 (n=28).

### Example 2. Expression of npr1(SAL) Increases Biotic Stress Resistance

It was also tested if npr1(SAL) expression could increase survival under pathogen effector-triggered (ETI) cell death (FIG. 2). First, the sub-cellular localization of the npr1(SAL)-GFP was confirmed in the surviving cells at the boundary of the cell death/cell survival during effector-triggered cell death. It was found that npr1(SAL)-GFP formed condensates in cells adjacent to the cell death region, but showed diffuse localization in cells distant from the death zone (FIG. 2 (Panel a)). Formation of NPR1 condensates during ETI was previously shown to be dependent on SA, confirming that npr1(SAL)-GFP exhibited similar response to SA relative to wild-type NR1 protein. However, unlike wild-type NPR1 protein, the npr1(SAL) could be detected at the cell death/survival boundary in transgenic plants. Next, the course of cell death in the same transgenic plants was measured and it was found that compared to NPR1-GFP, the npr1(SAL)-GFP-expressing plants showed enhanced cell survival against pathogen-induced cell death (FIG. 2 (Panel b)). These results demonstrate that npr1(SAL) expression either suppresses cell death and/or promotes survival against cell death induced by pathogen.

FIG. 2 illustrates the survival phenotype of npr1(SAL)-expressing plants under pathogen-triggered cell death: (A) Subcellular localization of npr1(SAL)-GFP or NPR1 during ETI. Three-week-old npr 1 (SAL)-GFP/npr1-7 transgenic plant was infected at the tip with Psm ES4326/AvrRpt2 to induce cell death. At 24 hpi, tissue was sampled from the cell death-survival boundary and GFP signal was collected from surviving cells located adjacent to- and distant from the cell death zone. Scale bar=20 μm. (B) Three-week-old NPR1-GFP/npr1-7, npr1(SAL)-GFPInpr1-7, npr1-7, and rps2 plants were treated with 1 mM SA 24 hr before inoculation with Psm ES4326/AvrRpt2 at OD600nm =0.02. Cell death was assessed at 1 dpi by ion leakage measurement using conductivity assay.

The results show that expression of npr1(SAL) in crop plants can enhance their survival against not only pathogen-induced cell death. The results further indicate that expression of npr1(SAL) in crop plants can also enhance survival against environmental (abiotic) stress-associated cell death. Unlike overexpression of wild-type NPR1, expression of Nprl (SA) provides immune gene expression and resistance while maintaining improved or normal plant growth.

### Example 3. Analysis of NPR1 and npr1(SAL) Structure and Function

NPR1 is a master regulator of the defense transcriptome induced by the plant immune signal salicylic acid. Despite the important role of NPR1 in plant immunity, understanding of its regulatory mechanisms has been hindered by a lack of structural information. Here we report cryo-electron microscopy and crystal structures of Arabidopsis NPR1 and its complex with the transcription factor TGA3. Cryo-electron microscopy analysis reveals that NPR1 is a bird-shaped homodimer comprising a central Broad-complex, Tramtrack and Bric-à-brac (BTB) domain, a BTB and carboxyterminal Kelch helix bundle, four ankyrin repeats and a disordered salicylic-acid-binding domain. Crystal structure analysis reveals a unique zinc-finger motif in BTB for interacting with ankyrin repeats and mediating NPR1 oligomerization. We found that, after stimulation, salicylic-acid-induced folding and docking of the salicylic-acid-binding domain onto ankyrin repeats is required for the transcriptional cofactor activity of NPR1, providing a structural explanation for a direct role of salicylic acid in regulating NPR1-dependent gene expression. Moreover, our structure of the TGA32-NPR12-TGA32 complex, DNA-binding assay and genetic data show that dimeric NPR1 activates transcription by bridging two fatty-acid-bound TGA3 dimers to form an enhanceosome. The stepwise assembly of the NPR1-TGA complex suggests possible hetero-oligomeric complex formation with other transcription factors, revealing how NPR1 reprograms the defense transcriptome.

As a positive regulator that is required for systemic acquired resistance mediated by salicylic acid (SA) in plants, the NONEXPRESSOR OF PATHOGENESIS-RELATED GENES 1 (NPR1) gene was identified more than two decades ago in Arabidopsis. Since then, the central role of NPR1 in controlling plant responses against a broad spectrum of pathogens has been firmly established. Notably, overexpression of Arabidopsis NPR1 in a wide range of plant species, including many crops, has been shown to enhance disease resistance against a variety of pathogens and stresses, indicating that the protein has intrinsic properties underlying its broad protective functions. Despite its importance in plant immunity and potential agricultural application, the molecular architecture of NPR1 has remained unclear, hindering mechanistic understanding of its regulation and function.

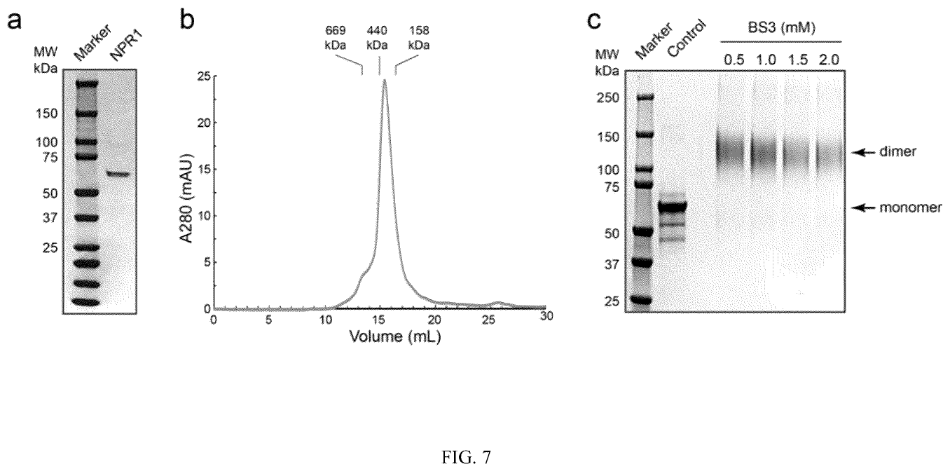

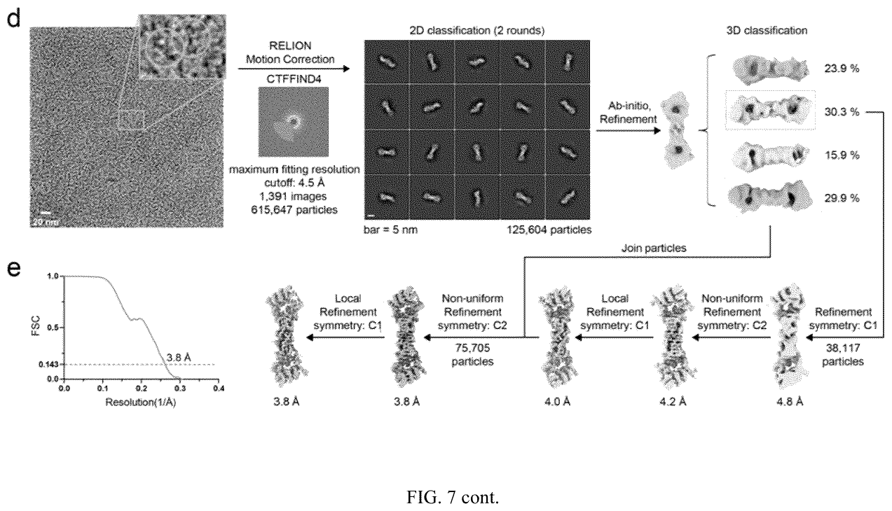

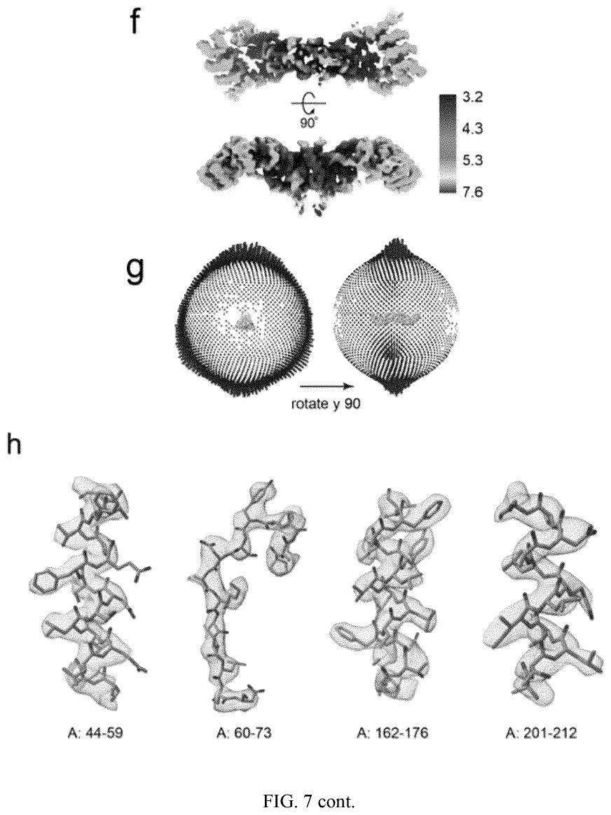

A. The cryo-EM structure of apo NPR1. As the first step towards structural analysis of NPR1, we expressed and purified the full-length Arabidopsis NPR1 using insect cells. Interestingly, this ˜66 kDa protein eluted between standards of 440 kDa and 158 kDa in size-exclusion chromatography (FIG. 7 (Panels a and b)). Treatment of NPR 1 with the cross-linking agent BS3 yielded predominantly a dimer band on the SDS-polyacrylamide gel electrophoresis (SDS-PAGE) gel (FIG. 7 (Panel c)). As NPR1 was purified in the presence of 2 mM dithiothreitol (DTT) without SA, our result indicates that dimeric NPR1 represents its reduced apo form.

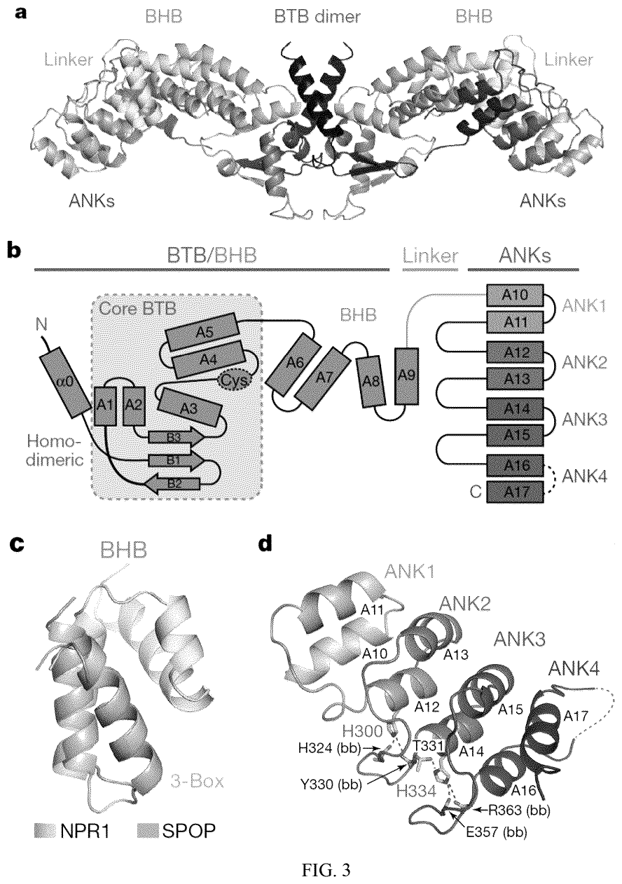

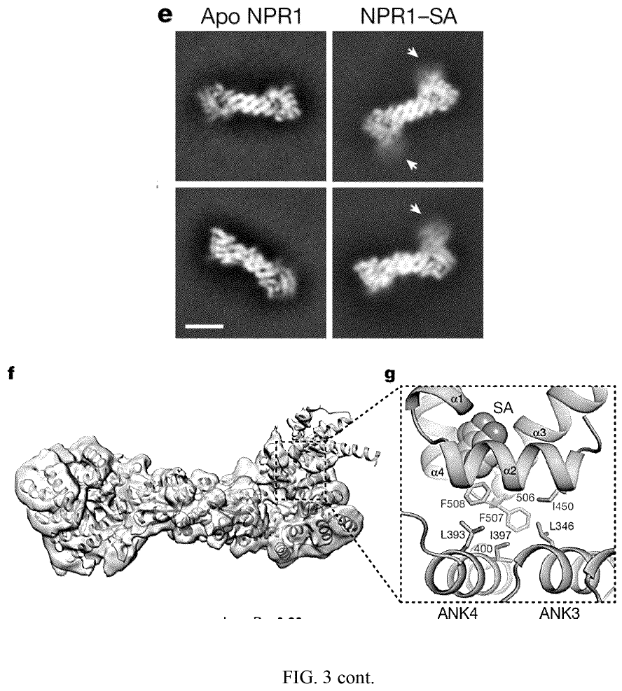

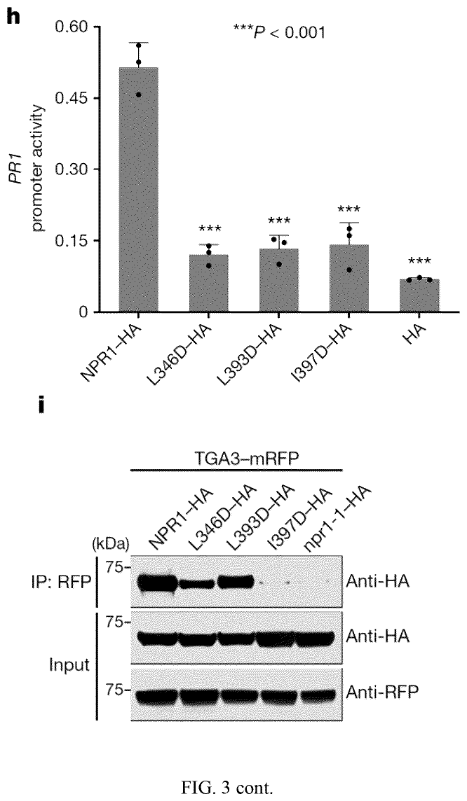

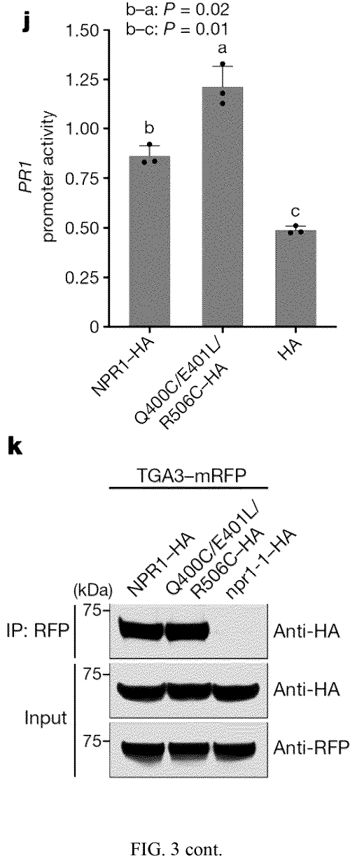

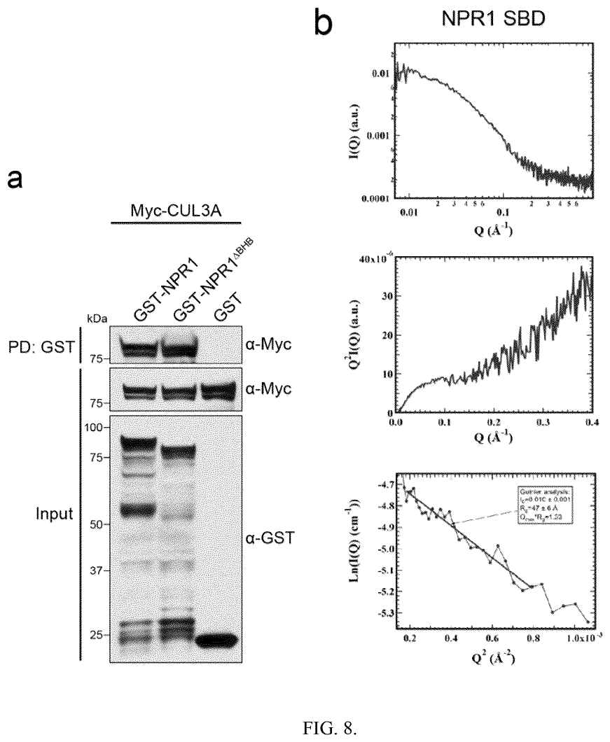

The structure of the NPR1 dimer was determined using single-particle cryo-electron microscopy (cryo-EM) at a resolution of 3.8 Å (FIG. 7 (Panels d-h)). The overall shape of the protein resembles a gliding bird (FIG. 3 (Panel a)). At the center of the dimer lies the core BTB domain, which consists of a three-stranded B-sheet (B1-B3) flanked by five helices (A1-A5; FIG. 3 (Panel b)). Similar to other homodimeric BTB domains, the NPR1 BTB domain contains an additional dimerization helix at the N terminus (α0; FIG. 3 (Panels a and b)); however, it lacks a domain-swapped B-strand preceding the dimerization helix in these proteins. C terminally to the BTB domain, NPR1 contains a unique four-helix bundle that shares the helical pair repeat pattern with the BTB and carboxyterminal Kelch (BACK) domain, but is much shorter than BACK (4 versus 8 helices) and is therefore named the BACK helix bundle (BHB). The first pair of helices in the BHB superimpose very well with the CUL3-binding box (3-box) in the ubiquitin ligase adaptor speckle-type POZ protein (SPOP) (FIG. 3 (Panel c)). Similar to SPOP, the removal of the BHB does not abolish the CUL3A interaction (FIG. 8 (Panel a)).

After the BHB is an extended linker loop connecting the BHB to ankyrin repeats (ANKs) (FIG. 3 (Panels b and d)). NPR1 contains three well-defined ANKs and a non-canonical fourth ANK with two a-helices linked by a 16-residue loop (FIG. 3 (Panel d)). The last three ANKs are connected by characteristic β-hairpins, whereas the first two ANKs are joined by a loop (FIG. 3 (Panel d)). These four ANKs pack against the central BTB/BHB through the first helices of individual ANKs, leaving the second ANK helices solvent exposed. A characteristic feature of ANKs is the presence of conserved histidine residues bridging neighboring repeats. Similarly, His300 of ANK2 and His334 of ANK3 form extensive hydrogen bonds with residues from the neighboring ANK3 and ANK4 units (FIG. 3 (Panel d)). Genetic mutations of these histidine residues (nim 1-2, H300Y; npr1-1, H334Y) cause a loss of PATHOGENESIS-RELATED (PR) gene expression and profound defects in systemic acquired resistance.

Beyond the ANKs, no density can be observed for the C-terminal putative SA-binding domain (SBD), consistent with it being disordered as revealed by small-angle X-ray scattering (SAXS) measurements (FIG. 8 (Panel b)).

B. NPR1 contains a zinc finger. As EM densities in some functionally important regions of NPR1 did not provide sufficient resolution for confident model building, we crystallized NPR1 (ASBD) (Thr39-Asp410), which diffracted to 3.06 Å (Extended Data Table 2). X-ray fluorescence scanning revealed the presence of Zn²⁺ ions in the protein crystal, but not in the buffer (FIG. 9 (Panel a)). Accordingly, analysis of the crystal structure revealed a distinct C₂HC zinc finger within a functionally important cysteine cluster that is conserved in NPR1 and its homologs, but is absent in mammalian BTB domains (FIG. 9 (Panel b)). The Zn²⁺ ion is coordinated by Cys150, Cys155, His157 and Cys160 in a consensus sequence of C-X-D-X_{2/3}C-X-H-X₂-C located within the A3-A4 loop of the BTB domain (FIG. 4 (Panels a and b)), where bold letters and Xs indicate conserved and non-conserved residues, respectively. Consistent with previous functional studies, we found that npr1(C150A), npr1(C150Y; npr1-2), npr1(C155A), npr1(C155Y; npr1-35) and npr1(C160A) are defective in their interaction with the TGA3 transcription factor and transcriptional activity when transiently expressed in Nicotiana benthamiana (FIG. 4 (Panels c and d). Notably, the npr1(H157A) mutation did not affect the activation of the PRI promoter (FIG. 4 (Panel d)), suggesting that npr1(H157A) may partially retain its ability to form a zinc finger, similar to the reported zinc-finger mutant of the HIV-1 nucleocapsid protein.

As part of its role in maintaining the structural integrity, the zinc finger bridges the BTB domain to ANK4 through a network of polar interactions (FIG. 4 (Panel a, inset)). The carboxylate group of Asp152, the most conserved residue within the zinc finger besides the zinc-coordinating residues, forms two hydrogen bonds with side chains of Lys370 and Gln371 of ANK4. The zinc-finger-ANK4 interaction is also ‘zip-locked’ by backbone hydrogen bonds involving Glu149 and Ala151 from the zinc finger, and Lys370, Thr373 and Ala375 from ANK4. As Lys370 is located at the last turn of the ANK helix, the Ala151-Lys370 backbone hydrogen bond is fortified by the helix dipole effect. Supporting the functional importance of the zinc finger-ANK4 interaction, simultaneously mutating Asp 152 to a basic residue (D152R) and disrupting the backbone hydrogen bond of Ala151 (A151P) in npr1(A151P/D152R) abolished the TGA3 interaction and PRI induction (FIG. 4 (Panels c and d)).

Moreover, the formation of the zinc finger, with side chains of Cys155 and His157 pointing inwards to coordinate Zn²⁺, pivots Cys156 directly into solvent (FIG. 4 (Panel a, inset)). Our previous study has shown that NPR1 in the quiescent state forms a homo-oligomer that is held together by intermolecular disulfide bonds, whereas, in response to SA induction, NPR1 is released through the activity of thioredoxins to trigger systemic acquired resistance. Notably, we found that, in the crystal structure, the protein lattice is formed by head-to-head stacking of two NPR1 dimers, and the pair of Cys156 residues from each NPR1 dimer are juxtaposed against their counterparts in the tetramer (FIG. 4 (Panel e)). Similar to our previous study of the NPR1 BTB domain, we found that hydrogen peroxide treatment of the NPR1 BTB-BHB domain (Thr39-Lys262) readily yielded dimer, trimer, and tetramer bands on the non-reducing SDS-PAGE (FIG. 4 (Panel f)). The NPR1 C156A mutation strongly reduced dimer formation and eliminated oligomer bands (trimer and tetramer), supporting Cys156 as a redox sensor. Within the NPR1 dimer at the monomer-monomer interface lie two Cys82 residues also in proximity (FIG. 4 (Panel e)), and the C82A mutation reduced the formation of trimer and tetramer bands (FIG. 4 (Panel f)), consistent with our previous genetic data. By contrast, a C212V/C216A/C223L triple-mutant behaved similarly to the WT protein, suggesting that these remaining non-zinc-chelating cysteine residues in the BTB-BHB do not contribute to redox-mediated NPR1 oligomerization in vitro.

C. SA induces docking of the SBD onto ANK3 4. Even though NPR1 contains all of the SA-binding residues in the SA receptor NPR4, and binds to SA with a sub-micromolar dissociation constant (Kd) value, its binding capacity is low, suggesting that only a small percentage of the recombinant SBD has the ability to bind to SA in vitro, possibly due to the absence of a chaperone. To mitigate this problem, we refolded full-length NPR 1 in the presence of 0.2 mM SA and subjected it to extensive purification before preparing cryo-EM samples.

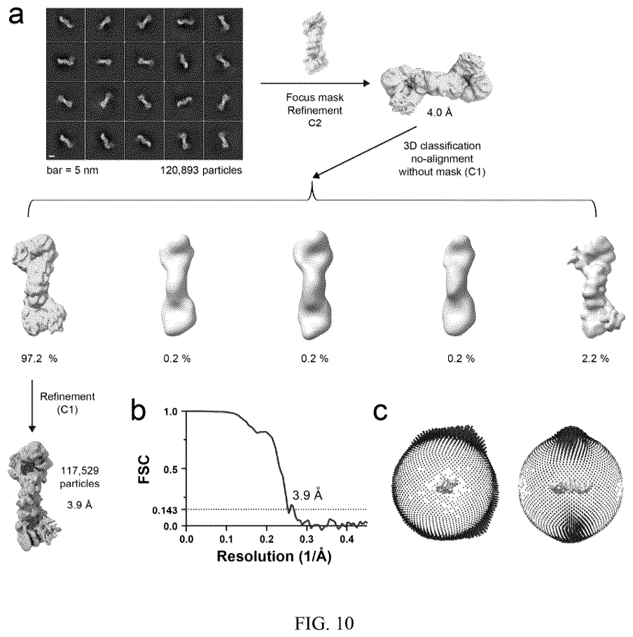

Analysis of cryo-EM 2D class averages of the NPR1-SA complex revealed similar features of the BTB-BHB-ANKs architecture as those in apo NPR1, confirming that NPR1 was properly refolded (FIG. 3 (Panel e), and FIG. 10). Notably, in the SA-bound state, additional densities were readily visible near the C termini of the ANKs of the NPR1 dimer (FIG. 3 (Panel e)), suggesting that SA induced the folding of the SBD.

The reconstructed 3D density map revealed that the SBD largely consists of helices (FIG. 3 (Panel f)), although the limited resolution in this region prevented detailed structural modelling. Aided by the recently reported structure of the NPR4 SA-binding core with a SA-containing four-helix bundle, we modelled the SBD using SWISS-MODEL and docked the structure onto the extra density of the NPR1-SA complex. In the structural model (FIG. 3 (Panel f)), the strongest density was observed in the α2-helix of the SBD (FIG. 3 (Panels f and g)). Although densities of the three ‘standing’ helices (α1, α3 and α4) are visible near the ANKs, they fade rapidly as these helices extend away from the ANKs (FIG. 3 (Panel f)). A well-defined hydrophobic interface is formed between Ile450 of the a2-helix and Phe507 and Phe508 at the conjunction of the α3-ζ4 helices of the SBD and Leu346, Leu393 and Ile397 of ANK3 and ANK4 (FIG. 3 (Panel g)). Notably, Leu346 is one of the three residues mutated in npr1(sim3) that block NPR1 SUMOylation and transcriptional activity. Similarly, the F507S and F508S mutations in Arabidopsis NPR1 or the corresponding residues (F505S and F506S) in tobacco (N. benthamiana) NPR1 abolish its binding to the transcription repressor NIMIN1/2. Effects caused by these mutations suggest that the observed interaction between SBD and ANKs might modulate the function of NPR1.

To test whether perturbation of the SBD-ANK interaction can affect NPR1 function, we mutated hydrophobic ANK residues at the interface (L346D, L393D and 1397D; FIG. 3 (Panel g)) and found that all three mutations diminished SA-induced PRI gene expression (FIG. 3 (Panel h)), even though two mutants (L346D and L393D) retained the interaction with TGA3 (FIG. 3 (Panel i)). We next tested whether locking the SBD-ANK interaction could boost the NPR1 function by introducing two cysteine residues, one in the SBD (R506C) and one in the ANKs (Q400C/E401L), to cross-link the SBD-ANK docking conformation through a disulfide bond. We found that the resulting mutant (Q400C/E401L/R506C) significantly elevated gene expression (FIG. 3 (Panel j)) without changing the interaction with TGA3 (FIG. 3 (Panel k)). Taken together, our results indicate that the SBD-ANK-docking conformation is required for NPR 1 in promoting defense gene transcription.

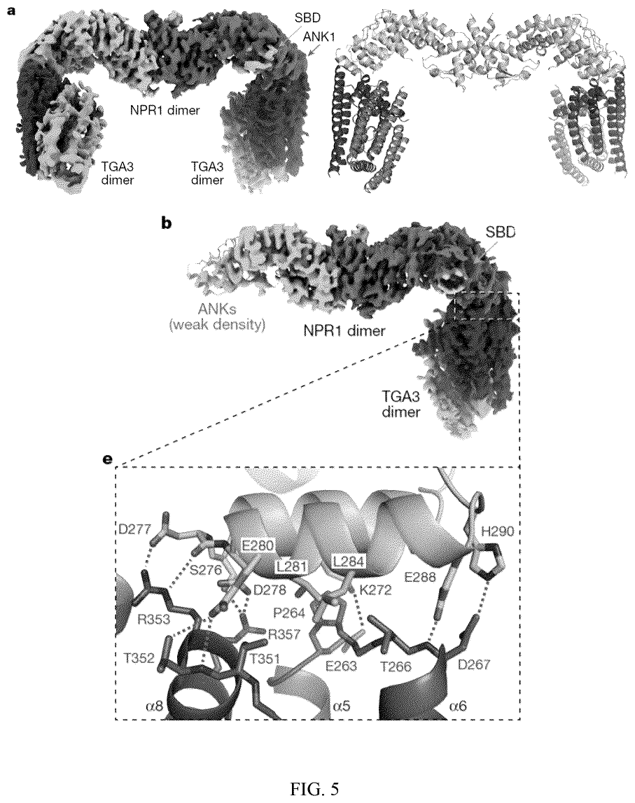

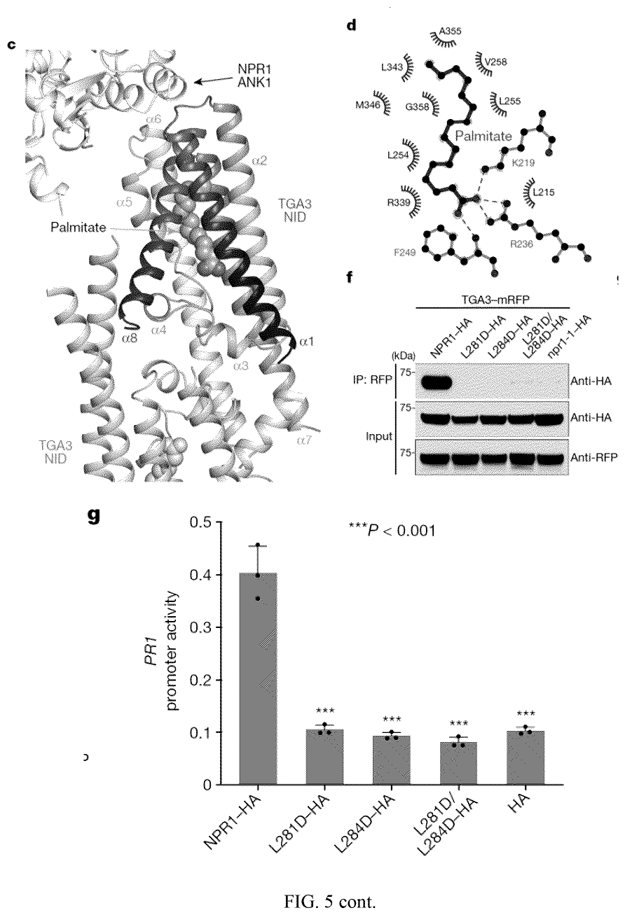

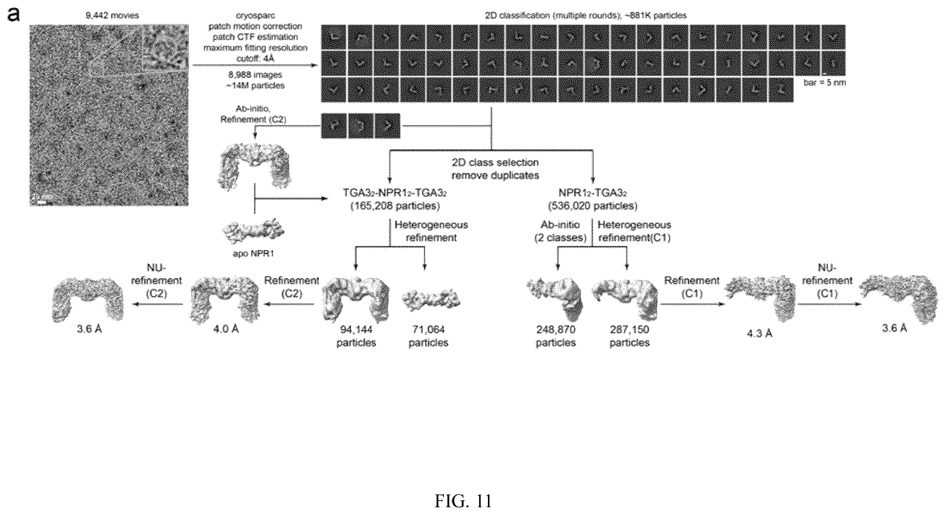

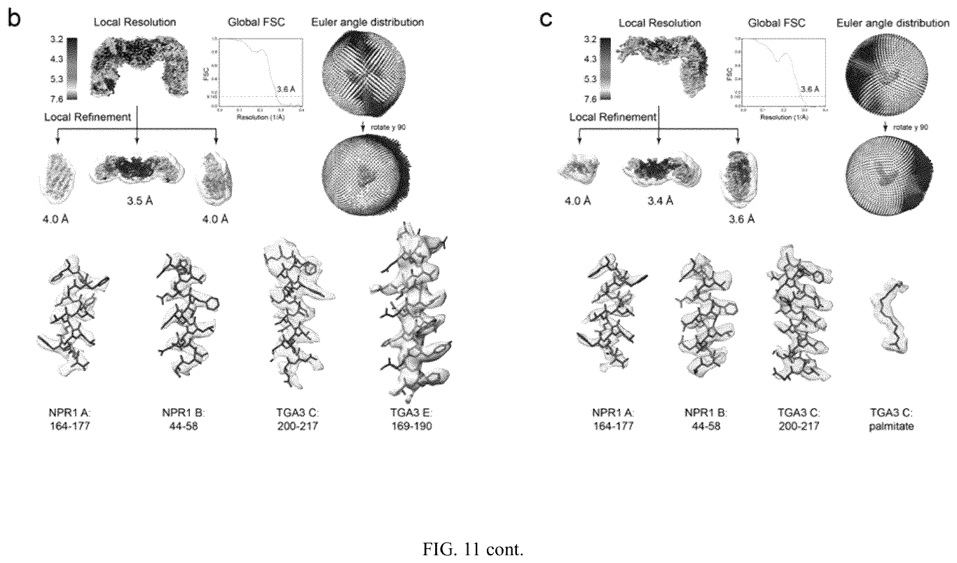

D. NPR1 bridges two TGA3 dimers through ANK1. To elucidate how SA-induced NPR1 activates TGA transcriptional activity, we determined the cryo-EM structure of NPR1 in complex with TGA3 in the presence of DNA carrying the TGA cis-element as-1 (LS7). Supporting the complex formation, extra densities are readily visible near ANK1 of NPR1 at one or both sides of the NPR1 dimer (FIG. 5 (Panels a and b)). Given the dimeric nature of NPR1, we hypothesize that the double-sided complex reflects the final assembly (FIG. 5 (Panel a)), whereas the single-sided complex reflects an assembly intermediate (FIG. 5 (Panel b)). Density maps of both complexes were reconstructed, each with an overall resolution of 3.6 Å (FIG. 11).

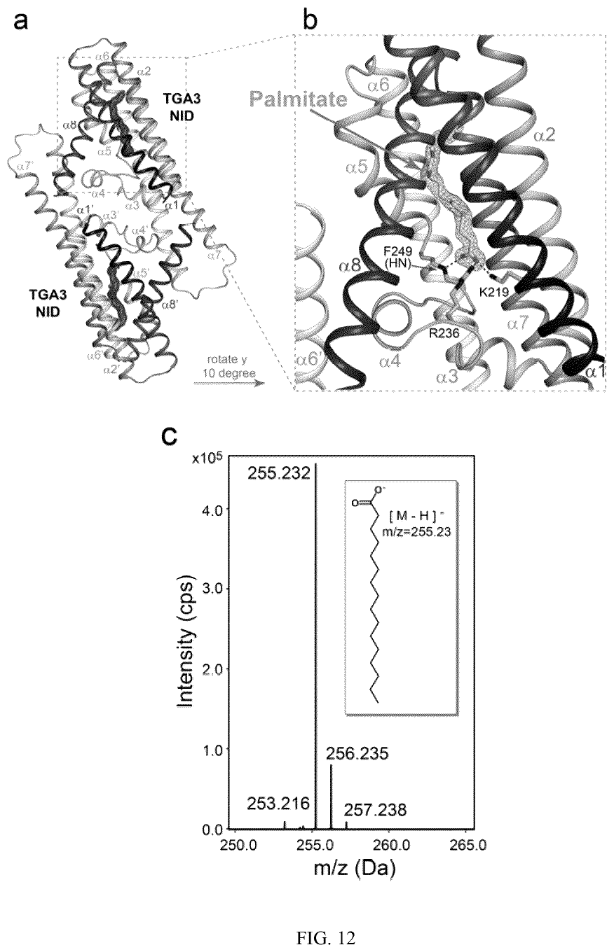

Surprisingly, EM analysis revealed that each extra density represents a dimer of the TGA3 C-terminal domain, which, to the best of our knowledge, has not been reported to form a stable dimer. Owing to its role of engaging NPR1, we named it the NPR1-interacting domain (NID) (FIG. 5 (Panels a-c)). The TGA3 NID contains five prominent long helices (α1, α2, α6, α7 and α8) and three short helices (α3, α4 and α5; FIG. 5 (Panel c)). Four long helices (α1, α2, α6 and α7) form an arched layer, whereas three short helices (α3, α4 and α5) zig-zag across the layer and support the C-terminal helix (α8), which tilts away from the arched layer. The NID dimer is formed through extensive interactions between the three short helices (α3, α4 and α5) and between the tilted α8 helix in one monomer and the α6-α7 helices in the other monomer (FIG. 5 (Panel c)). Unexpectedly, a palmitic acid was found inside the NID, an observation corroborated by the 1.5 Å resolution crystal structure and mass spectrometry (MS) analysis (FIG. 5 (Panel d)), FIG. 12). This observation further implicates lipid metabolism in regulating plant immunity.

NPR1-TGA3 binding is mediated by elaborate interactions (FIG. 5 (Panel e)). Surface-exposed hydrophobic residues from ANKI of NPR1, such as Leu281 and Leu284, project onto a hydrophobic concave surface of one TGA3 subunit formed by the α5-α6 loop (Pro264 and Thr266) and α7-α8 loop (Thr351), whereas numerous hydrogen bonds and salt bridges are observed between ANKI residues (Lys272, Ser276, Asp277, Asp278, Glu280, Glu288 and His290) and TGA3 NID residues from the α5-α6 loop (Glu263), α6 helix (Asp267), α8 helix (Thr352, Arg353 and Arg357). Corroborating these observations, mutating interfacial residues in NPR1, such as L281D and L284D, abolished the NPR1-TGA3 interaction (FIG. 5 (Panel f)) and SA-induced PRI gene expression (FIG. 5 (Panel g)).

In the NPR1 complex with TGA3, weak density of the SBD can also be visualized (FIG. 5 (Panels a and b). Despite the limited resolution, which prevents model building, it is evident that the SBD has no direct contact with TGA3. Notably, in the single-sided NPR12-TGA32 assembly intermediate, the unengaged NPR 1 subunit has much weaker densities in its ANKs and SBD than the subunit engaged in TGA3 binding (FIG. 5 (Panel b)), suggesting that the stability of ANKs may be dynamically regulated. Moreover, no density was observed for DNA or the basic-leucine zipper (bZIP) region of TGA3, suggesting that the DNA-binding domain is separated from the NID by a flexible linker.

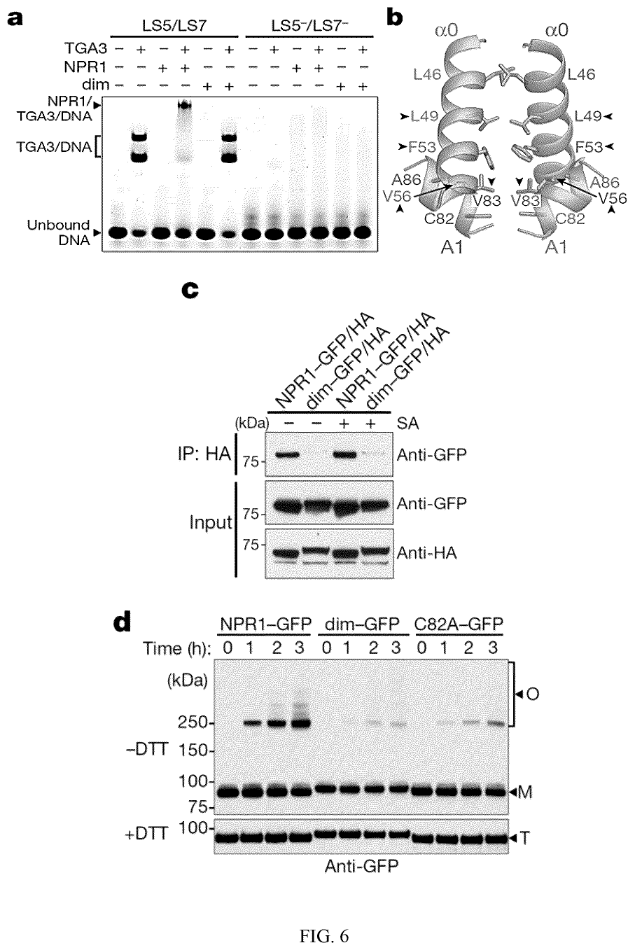

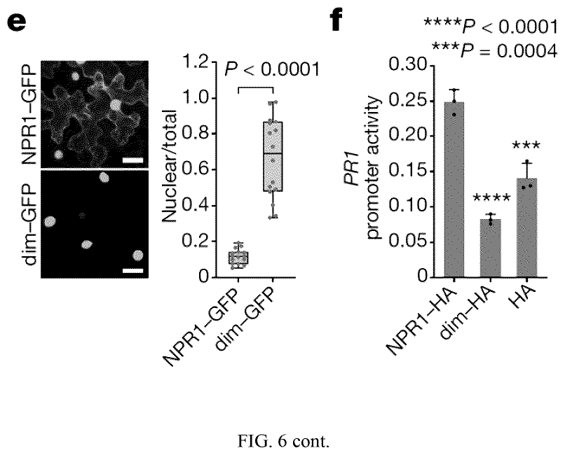

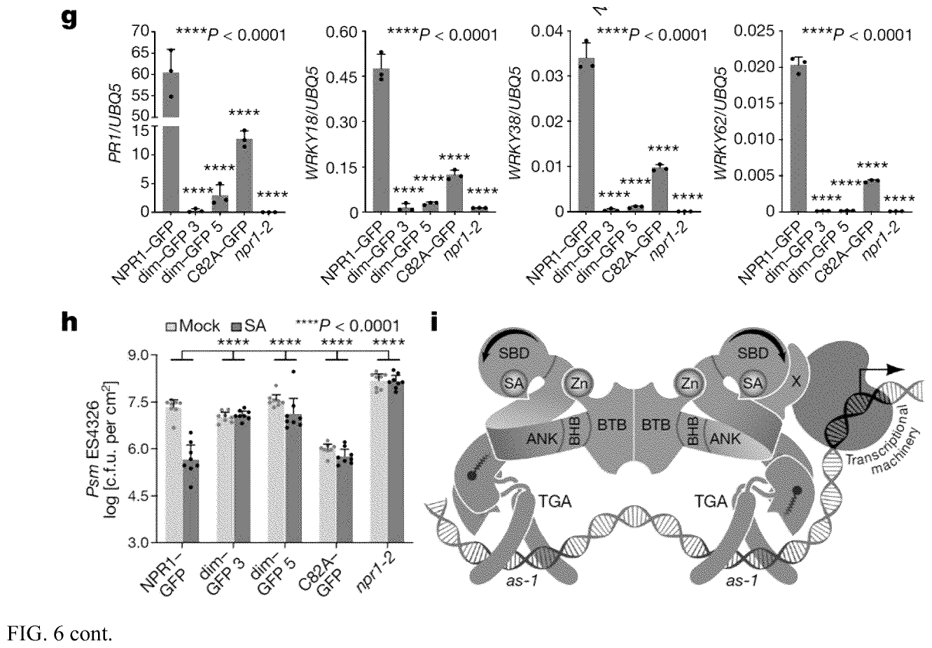

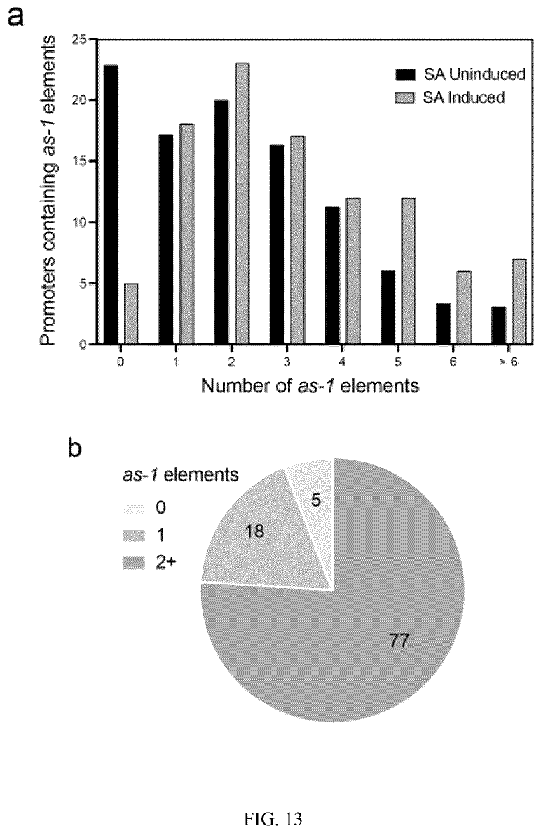

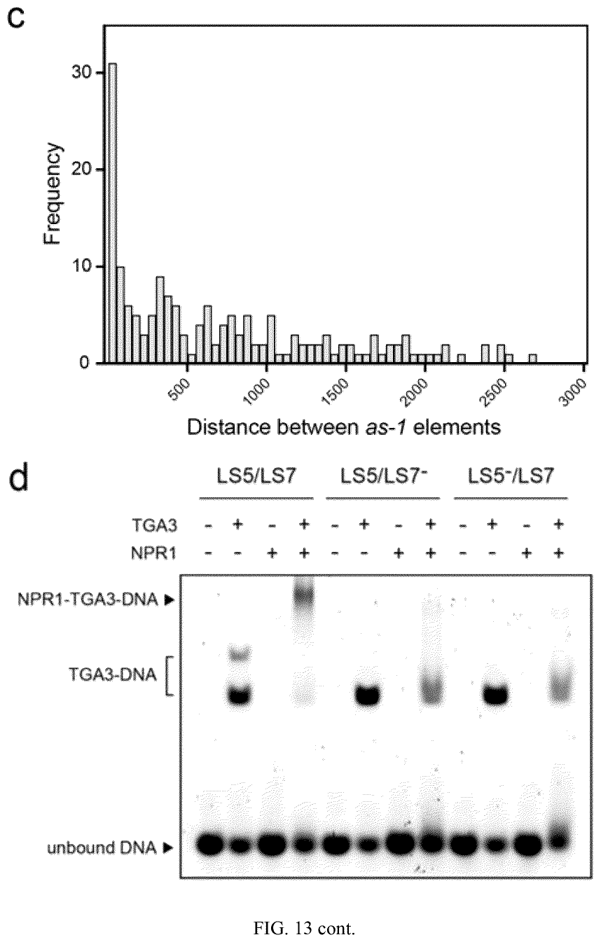

E. The NPR1 dimer is required in plant immunity. The structure of the TGA32-NPR12-TGA32 complex, with the dimeric NPR1 engaging two TGA3 dimers, suggests that NPR1 may serve as a transcription cofactor by bridging two transcription factor complexes in an enhanceosome. Consistent with this hypothesis, the as-/element is significantly enriched in the promoters of top 100 SA-induced genes, and 77 contain at least two as-/elements that are frequently separated by <50 bp (FIG. 13 (Panels a-c)). On the basis of this information, we conducted the electrophoresis mobility shift assay (EMSA) using the PRI gene promoter containing two as-/elements (LS5 and LS7), which are both required for NPR1-mediated transcription in a reporter assay. The addition of TGA3 yielded two upshifted bands of the promoter DNA, corresponding to single and double occupancy of the as-/elements, but not the negative-control DNA with both as-/elements replaced by adenosines (FIG. 6 (Panel a)). Although NPR1 itself did not bind to DNA, it completely supershifted the TGA3 double-occupancy band and depleted the single-occupancy band. Moreover, mutating either of the two as-1 elements in the DNA blocked the NPR1-mediated supershift band (FIG. 13 (Panel d)). These results support our hypothesis that the NPR1 dimer activates TGA transcription factor activity by bridging two DNA-bound TGA complexes.

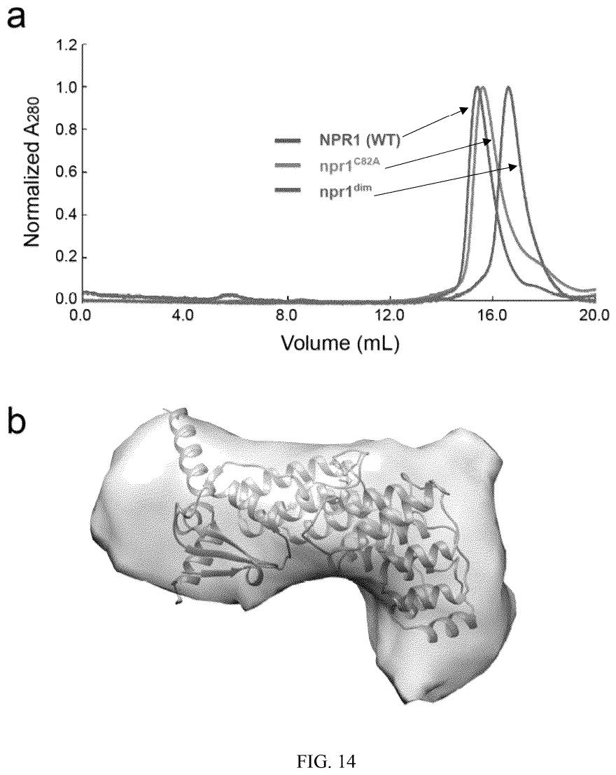

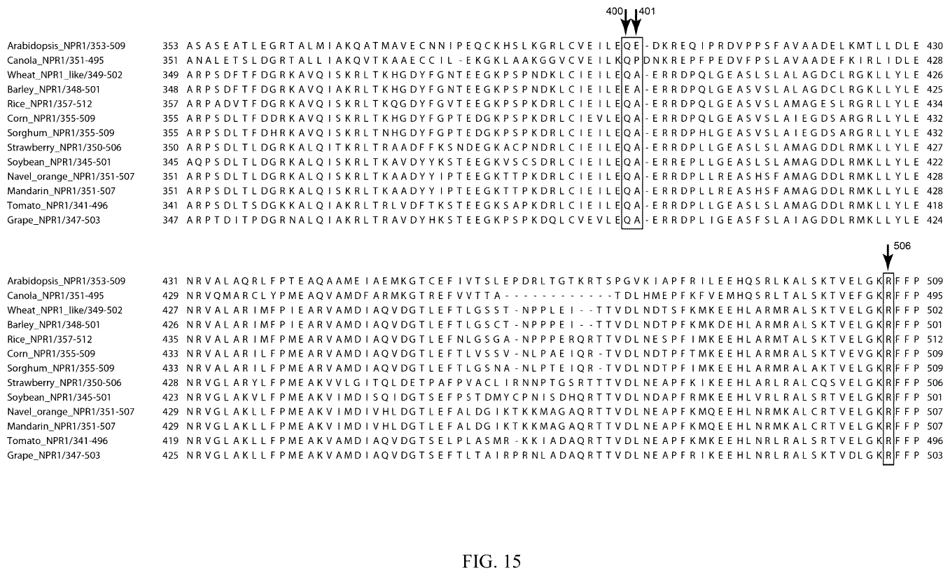

To test our hypothesis genetically, we designed a dimerization-deficient npr1(dim) mutant by introducing L49D/F53D/V56D/V83K mutations into the α0 and A1 helices of the NPR1 BTB dimer interface (FIG. 6 (Panel b)), following a similar strategy to disrupt the dimerization of the SPOP BTB domain. The npr1(dim) mutant not only failed to self-interact (FIG. 6 (Panel c)), but also eluted off the size-exclusion column at a later volume than WT NPR1 (FIG. 14 (Panel a)). A low-resolution cryo-EM map of npr1(dim) revealed a single-wing shape, confirming that the mutations disrupted dimerization without affecting the overall protein architecture (FIG. 14 (Panel b)).

Before testing the transcriptional activity of npr1 (dim), we evaluated its ability to form a quiescent homo-oligomer, which has been shown to prevent NPR1 nuclear translocation. We found that npr1(dim) was deficient in oligomer formation and had increased nuclear accumulation (FIG. 6 (Panels d and e)), similar to the npr1(C82A) mutant. However, in contrast to npr1(C82A), which has constitutive PRI gene expression, npr1(dim) was unable to induce the PRI promoter when transiently expressed in N. benthamiana (FIG. 6 (Panel f)), probably because it is unable to cross-link the TGA complexes (FIG. 6 (Panel a)). Similarly, in stable trans-genic lines, npr1(dim)-GFP failed to activate PRI, WRKY18, WRKY38 and WRKY62 (FIG. 6 (Panel g)), all of which have at least two as-/elements in their promoters (FIG. 13 (Panel b)). As a consequence, in contrast to WT plants, the npr1(dim)-GFP plants were compromised in SA-induced defense against Pseudomonas syringae pv. maculicola ES4326, whereas the npr1(C82A) mutant, which is able to dimerize (FIG. 14 (Panel a)), conferred constitutive resistance (FIG. 6 (Panel h)). These results establish the NPR1 homodimer as the functional unit that is required not only for oligomer formation in the quiescent state, but also for NPR1 function in inducing defense gene expression and resistance against pathogen challenge.

This study establishes a structural framework for explaining data from over two decades of NPR1 studies (FIG. 6 (Panel i)). The lack of a direct contact between SBD and TGA suggests that the SBD-ANK-docking conformation may enable post-translational modification, such as SUMOylation, that is required for NPR1 activity, and/or facilitate the recruitment of an unknown transcription regulator. The need for investigating new regulatory factors is reinforced by our unexpected observation of a palmitic acid within the TGA3 NID. The structural elucidation of the TGA32-NPR12-TGA32 complex and its assembly intermediate raises the possibility that NPR1 reprograms the defense transcriptome in response to a wide range of biotic and abiotic stresses through the engagement of not only TGA homodimers, but also heterologous transcription activators, suppressors, and lipid metabolites in an enhanceosome complex (FIG. 6 (Panel i)).

### Methods

A. Cryo-EM Studies of apo NPR1, NPR1-SA and the npr1(dim) Mutant

1. Cloning, expression and purification using insect cells. Full-length Arabidopsis thaliana NPR1 and the corresponding npr1 (dim) mutant (L49D/F53D/V56D/V83K) were cloned into the pFASTBac1 vector as a His10-GST-NPR1-Strep fusion protein containing a PreScission pro-tease site after GST. Baculovirus was generated and amplified according to the manufacturer's protocol (Thermo Fisher Scientific) using Sf9 cells (Expression Systems). For protein expression, Trichoplusiani (High Five) cells were cultured in the ESF 921 insect cell culture medium (Ex-pression Systems) at 27° C.; the suspension cell culture was infected with a high-titer baculovirus stock and collected after 50 h by centrifugation at 200 g for 5 min. Cell pellets were resuspended and lysed by sonication in the purification buffer (25 mM HEPES pH 7.5 and 150 mM NaCl) and supplemented with protease inhibitors (15 μM leupeptin, 1 μM pepstatin A, 2 μM E-64, 0.1 μM aprotinin, 1 mM phenylmethylsulphonyl fluoride) and DNase I. After centrifugation at 15,000 rpm and 4° C. for 15 min, the fusion protein was purified using the Strep-Tactin Superflow Plus resin (Qiagen). After PreScission protease cleavage to remove the His10-GST tag, the WT protein or the npr l (dim) mutant were further purified by passing it through the Talon resin (Takara Bio) and eluted from the column in the presence of 10-15 mM imidazole in the lysis buffer, whereas the His10-GST tag was retained on the cobalt column.

WT NPR1 was concentrated and further purified by size-exclusion chromatography using the Superose 6 increase 10/300 GL column (Cytiva) pre-equilibrated with the purification buffer containing 2 mM DTT. Peak fractions were concentrated and cross-linked using 2 mM BS3 (Thermo Fisher Scientific) at room temperature for 30 min, and the cross-linked sample was further purified by size-exclusion chromatography (Superose 6 increase 10/300 GL; Cytiva) for cryo-EM grid preparation.

The npr1(dim) mutant used for cryo-EM grid preparation was purified using identical procedures as the WT NPR1.

The SA-bound NPR1 was obtained by refolding NPR1 in the presence of SA. In brief, purified NPR1-Strep was concentrated and dialyzed against 8 m urea followed by dialysis in the refolding buffer containing 25 mM HEPES (pH 7.5), 150 mM NaCl, 2 mM DTT and 0.2 mM SA. The refolded sample was purified by size-exclusion chromatography using the Superose 6 increase 10/300 GL column (Cytiva) pre-equilibrated with the refolding buffer. The peak fraction was then concentrated and cross-linked with 2 mM BS3 as described above; the cross-linked sample was repurified by size-exclusion chromatography (Superose 6 increase 10/300 GL; Cytiva) for cryo-EM grid preparation.

2. Cryo-EM sample preparation and data collection. The cryo-EM grids containing NPR1 were prepared using the Leica EM GP Automatic Plunge Freezer at 10° C. and 95% humidity. The UltrAuFoil R1.2/1.3 300-mesh grids were glow-discharged using the Tergeo-EM plasma cleaner (Pie Scientific). A 3 μl sample of BS3 cross-linked NPR1 (˜0.6 mg ml-1) was applied to the grids and blotted for 2-3 s with Whatman No. 1 filter paper (Whatman International) to remove excess sample and plunge-frozen into liquid ethane cooled by liquid nitrogen. A total of 1,638 micrograph stacks were collected with SerialEM35 on a Talos Arctica microscope at 200 kV equipped with a K2 Summit direct electron detector (Gatan), at a nominal magnification of 45,000× and defocus values from −2.5 μm to −0.8 μm, yielding a pixel resolution of 0.9317 Å px-1. Each stack was exposed for 8.4 s with an exposing time of 0.14 s per frame, resulting in 60 frames per stack. The total dose was approximately 60 e-Å-2 for each stack.

The cryo-EM grids containing NPR1-SA were prepared similarly to that of NPR1. A total of 9,486 micrograph stacks were collected with SerialEM on a Titan Krios microscope (Thermo Fisher Scientific) at 300 kV equipped with a K3 direct electron detector (Gatan), at a nominal magnification of 64,000× and defocus values from −2.5 μm to −0.8 μm, yielding a pixel resolution of 0.5325 Å px-1. Each stack was exposed for 8.069 s with an exposing time of 0.134 s per frame, resulting in 60 frames per stack. The total dose was approximately 60 e-Å-2 for each stack.

The cryo-EM grids containing the npr1 (dim) mutant were prepared similarly except that C-flat R1.2/1.3 300-mesh grids (Protochips) coated with gold were used. A total of 3,500 movies were recorded with a defocus range from −2.0 μm to −0.8 μm on the Talos Arctica electron microscope (Thermo Fisher Scientific) at 200 kV equipped with a K3 Summit direct electron detector (Gatan) at a nominal magnification of 45,000× (0.88 Å px-1) using SerialEM. Each stack was exposed for 2.716 s with an exposing time of 0.045 s per frame. The total dose was approximately 55 e-Å-2 distributed over 60 frames.

3. Cryo-EM data processing and model building. For cryo-EM data analysis of apo NPR1, movie alignment was performed in RELION36 and the parameters of the contrast transfer function (CTF) were determined on the motion corrected sum of frames using CTFFIND4.1 (ref. 37). Micrographs (1,391) were selected on the basis of the CTF fit resolution using a cut-off of 4.5 Å. A total of 615,647 NPR1 particles were boxed out using template-free particle picking. Two consecutive rounds of 2D classification were performed to clean the extracted particles. A total of 125,604 clean particles were used for further processing. The processing flow chart is shown in FIG. 7 (Panel d). In brief, a consensus reconstruction obtained with cryoSPARC using all of the clean particles resulted in a map at ˜6 Å resolution. Four ab initio 3D references were generated and processed for multi-reference 3D classification using heterogeneous refinement. A subset of 38,117 particles showing the best structural features was selected and processed for homogeneous refinement without applying symmetry. Non-uniform refinement using C2 symmetry yielded a reconstruction with an overall resolution of 4.2 Å. The resolution improved to 4.0 Å after one round of local refinement. At this point, another subset of 37,588 particles identified from the previous heterogeneous refinement were added and processed for a new round of non-uniform refinement using C2 symmetry, yielding a 3.8 Å reconstruction. A focus refinement extended the resolution of the center domain of the dimer to 3.6 Å. The de novo model building for apo NPR1 was conducted using COOT. The region containing the cysteine cluster (Glu149-Cys160) was initially built as polyalanines due to the limited local resolution. This region was rebuilt on the basis of the knowledge of a zinc finger from crystal structure analysis of NPR1. Structure refinement was performed using PHENIX in real space with secondary and geometry restraints.

The NPR1-SA dataset was processed in a similar manner to that described above. A total of 5,424 micrographs were selected on the basis of the CTF fit resolution with a cut-off value of 4.5 Å. Around 5.6 million particles were used for 2D classification after curating the template-free blob picking results. After three rounds of cleaning with 2D classification, 120,893 particles were selected and imported back to RELION for refinement. The apo NPR1 map and mask were used as the 3D reference for the consensus refinement with C2 symmetry focusing on the NPR1 dimer core domain, which resulted in a 4.0 Å map. 3D classification without alignment was conducted (no mask or symmetry applied). One good class containing 117,529 particles was selected and processed for local refinement with a shape mask yielding an overall resolution of 3.9 Å. A summary of the processing flow chart is shown in FIG. 10.

For the npr1 (dim) dataset, movie alignment and the CTF estimation were conducted using cryoSPARC. A total of 2,377 micrographs were selected based on the CTF fit resolution cut-off of 4 Å. Manually picked particles were used as input for Topaz particle picking41. Rounds of 2D classification were performed to isolate good classes. Two ab initio models were generated and processed a subset of 18,795 particles showing better structural features was selected for homogeneous refinement.

4. Cloning, overexpression and purication of the NPR1 SBD for biological SAXS. The Arabidopsis NPR1 SBD (Pro412-Arg593) was cloned into the pFASTBacl vector as a His10-GST-NPR1-Strep fusion protein containing a PreScission protease site following GST. The baculovirus generation, protein expression and purification followed similar procedures as described for NPR1. The protein was concentrated to 1 mg ml-1 in a buffer containing 25 mM HEPES (pH 7.5), 150 mM NaCl and 2 mM DTT for biological SAXS measurements at beamline 12-ID-B of the Advanced Photon Source (APS).

### B. Cryo-EM Studies of the NPR1 TGA3 Complex

1. Expression and purification of Arabidopsis NPR1 and TGA3 using Escherichia coli. Full-length Arabidopsis NPR1 was cloned into the pTrcHis vector (Thermo Fisher Scientific) as a His10-MBP-fusion protein containing a PreScission protease site following MBP and a C-terminal Strep tag (Strep3) with the last Lys residue of the Strep tag mutated to Arg. Transformed BL21 (DE3) E. coli cells were grown in Luria-Bertani (LB) medium at 37° C. to an optical density at 600 nm (OD600) of 0.5, induced with 0.1 mM IPTG, 0.1 mM ZnSO4 and 0.2 mM SA at 18° C. overnight. The purification of the E. coli-expressed NPR1 was performed as described above for the insect-cell-expressed NPR1, except that the purification buffer contained 0.2 mM SA.

Arabidopsis TGA3 (Asn87-Thr384) was cloned into the pTrcHis vector (Thermo Fisher Scientific) as a His10-MBP fusion protein containing a PreScission protease site following MBP and a C-terminal tandem Flag-HA tag. Transformed BL21 (DE3) E. coli cells were grown in LB medium at 37° C. to an OD600 of 0.5 and induced with 0.1 mM IPTG at 18° C. overnight. The purification was performed as described above for NPR1.

2. NPR1 TGA3 LS7-DNA cryo-EM sample preparation. The purified NPR1-Strep3 (20 μM), TGA3-Flag-HA (48 μM) and the LS7 DNA containing a palindromic TGA-binding site (underlined in 5′-CACTATTTTACTGACGTCATAGATGTGGCG-3′, 57.6 μM) were mixed and incubated at 4° C. for 30 min. The protein-DNA complex was cross-linked with 1.5 mM BS3 (Thermo Fisher Scientific) at room temperature for 30 min and quenched with 50 mM Tris (pH 8.0). The protein-DNA mixture was further purified using a Strep-Tactin column (Qiagen) and size-exclusion chromatography (Superose 6 in-crease 10/300 GL, Cytiva) in a buffer containing 25 mM HEPES (pH 7.5), 150 mM NaCl, 2 mM DTT and 0.2 mM SA.

3. Cryo-EM sample preparation and data collection. Cryo-EM grids containing the NPR1-TGA3-LS7-DNA complex were prepared using the Leica EM GP2 Automatic Plunge Freezer at 4° C. and 85% humidity. The UltrAuFoil R1.2/1.3 300-mesh grids (Quantifoil) were glow-discharged using the PELCO easiGlow Glow Discharge Cleaning System (Ted Pella). A 3 μl sample of NPR1-TGA3-LS7-DNA (˜1.3 mg/ml) was applied to the grid, incubated for 60 s in the chamber and blotted for 2.4 s with Whatman No. 1 filter paper (Whatman International) to re-move excess sample, and then plunge-frozen into liquid ethane cooled by liquid nitrogen. A total of 9,442 micrograph stacks were collected with SerialEM on a Titan Krios microscope (Thermo Fisher Scientific) at 300 kV equipped with a K3 direct electron detector (Gatan), at a nominal magnification of 81,000× and defocus values from −2.0 μm to −1.0 μm, yielding a resolution of 1.066 Åfipx-1. Each stack was exposed for 8.3 s with an exposing time of 0.138 s per frame, resulting in 60 frames per stack. The total dose was approximately 60 e⁻Å⁻² per stack.

4. Cryo-EM data processing and model building. For cryo-EM data analysis of TGA32-NPR12-TGA32, movie alignment and the CTF estimation were conducted using cryoSPARC38. A total of 8,988 micrographs were selected based on the CTF fit resolution using a cut-off value of 4.0 Å. A total of around 14 million particles were boxed out using the NPR1 as the template. Multiple rounds of 2D classification and selection were performed to clean the extracted particles. A total of around 881,000 clean particles were used for further processing. The processing flow chart is shown in FIG. 11 and summarized below. In brief, three classes with clear TGA3 densities on both sides of NPR1 were selected for ab initio reconstruction and homogeneous refinement with C2 symmetry of the TGA32-NPR12 TGA32 complex. A total of 165,208 particles that were selected from the clean set of particles were processed for 3D classification/heterogeneous refinement using apo NPR1 and the low-resolution TGA32-NPR12-TGA32 maps as the template. A subset of 94,144 particles featuring the TGA32-NPR12-TGA32 complex were processed for homogeneous refinement with C2 symmetry. Non-uniform refinement was then performed using C2 symmetry, yielding a reconstruction with an overall resolution of 3.6 Å. Focused refinements of NPR12 (central region) and two TGA32 (left and right regions) extended local resolutions to 3.5 Å and 4.0 Å, respectively. A composite map was generated using PHENIX40 for model building.

Similarly, 536,200 particles were selected for reconstruction of the NPR12-TGA32 complex. After ab initio reconstruction and 3D classification/heterogeneous refinement, 287,150 particles were selected for homogenous and non-uniform refinement, yielding an overall resolution of 3.6 Å. Focused refinement of NPR12 (excluding the weak ANK region), TGA32 and the weak ANK region extended local resolutions to 3.4 Å, 3.6 Å and 4.0 Å, respectively.

The de novo model building was conducted using COOT. Structure refinement was performed using PHENIX in real space with secondary structure and geometry restraints.

5. X-ray crystallography studies of NPR1 (ΔSBD)) and the TGA3 NID. Arabidopsis NPR1 (²SBD) (Thr39-Asp410) was cloned into the pTrcHis vector (Thermo Fisher Scientific) as a His10-MBP fusion protein containing a PreScission protease site following MBP and a C-terminal Strep tag. Expression and purification of NPR1 (²SBD) was performed using similar procedures to those described above for E. coli-expressed NPR1, except that the size-exclusion chromatography buffer contained 20 mM HEPES (pH 7.5), 150 mM NaCl and 1 mM TCEP. Protein crystals were grown using the sitting-drop vapor diffusion method at 20° C. Each drop was prepared by mixing 1 μl of the protein solution at 6 mg ml-1 with 1 μl of the reservoir solution containing 0.1 M HEPES (pH 7.5), 19% PEG 3350, 0.25 m tri-sodium citrate. Diffraction-quality protein crystals were collected after 1 week and cryoprotected with the reservoir solution containing 25% glycerol.

The Arabidopsis TGA3 NID (Ala160-Thr384) was cloned into the pTrcHis vector (Thermo Fisher Scientific) as a His10-MBP fusion protein containing a PreScission protease site following MBP and a C-terminal tandem Flag-HA tag. The purification procedure was per-formed similarly to that of Arabidopsis TGA3 (Asn87-Thr384) except that the size-exclusion chromatography buffer contained 20 mM HEPES (pH 7.5), 150 mM NaCl and 1 mM TCEP. Protein crystals were grown using the sitting-drop vapor diffusion method at 20° C. Each drop contained 1 μl of the protein solution mixed with 1 μl of the reservoir solution (0.1 m sodium acetate at pH 5.0, 20% (v/v) MPD). Diffraction quality protein crystals were collected after 1 week and cryoprotected with the reservoir solution also containing 25% glycerol.

X-ray fluorescence and diffraction datasets of NPR1 (²SBD) were collected at the Northeastern Collaborative Access Team (NECAT) 24-ID-C beamline at the Advanced Photon Source at the Argonne National Laboratory. The X-ray diffraction data were processed using XDS42. The phase information was obtained by molecular replacement with the PHASER module in the PHENIX suite using the cryo-EM coordinates of apo NPR1 and the TGA3 subunit in the NPR12-TGA32 complex as the search model for NPR1 (²SBD) and the TGA3 NID, respectively. Iterative model building and refinement were performed using COOT and PHENIX. For NPR1 (²SBD), the final model has 95.13% residues in the favored region, 4.87% in the allowed region and 0.0% in the outlier region of the Ramachandran plot. For the TGA3 NID, the final model has 99.01% residues in the favored region, 0.99% residues in the allowed region and 0.0% in the outlier region of the Ramachandran plot.

C. Lipid Extraction from the TGA3 NID and LC ESI MS Analysis
The fatty acid was extracted from the purified TGA3 NID protein using the two-phase Bligh-Dyer method. The extract was analyzed using normal-phase liquid chromatography-electrospray ionization/MS (LC-ESI/MS) in negative mode using an Agilent 1200 Quaternary LC system coupled to a high-resolution TripleTOF5600 mass spectrometer (Sciex) as previously described. Data acquisition and analysis were performed using the Analyst TF1.5 software (Sciex).
D. In vitro NPR1 Oxidation

WT NPR1 BTB/BHB (Thr39-Lys262) and the npr1(C156A), npr1 (C82A) and npr1(C212V/C216A/C223L) mutants were cloned into the pTrcHis vector (Thermo Fisher Scientific) as His10-MBP fusion proteins with a PreScission protease site following MBP and a C-terminal Strep tag. The proteins were expressed and purified similarly to the NPR1 (²SBD) as described above. The NPR1 BTB/BHB WT and mutant proteins at a concentration of 25 μM were incubated with 1 mM hydrogen peroxide in a buffer containing 125 mM HEPES and 150 mM NaCl at pH 8.0 at room temperature for 45 min. The oxidized samples were incubated with 50 mM iodoacetamide for 5 min, mixed with 4× SDS loading dye and analyzed using SDS-PAGE in the absence of any reducing reagent.

E. Promoter Analysis for as-1 Elements

For the promoter analysis, the scanMotifGenomeWide.pl function in HOMER (v.4.11) was initially used to generate a bed file containing all as-1 elements in the Arabidopsis genome. Moreover, using R (v.4.1.1) and the Araport 11 GTF, bed files for the 3,000 bp upstream regions of the top 100 induced and of all the uninduced genes in response to 8 h of treatment by the SA analogue benzothiadiazole were generated. The window function in bedtools (v.2.30.0) with a window of 0 bp was used to intersect both promoter and as-/element bed files to generate bed files containing all as-1 elements within the 3,000 bp upstream regions of the top 100 SA-induced genes and uninduced genes. The number of as-1 elements in each promoter was counted and the as-1-element count distribution of the top 100 SA-induced genes was compared to the distribution of uninduced genes using a Æ2 test. The promoters of the top 100 SA-induced genes were further analyzed for multiple as-1 elements and subset into groups containing 0, 1, 2 or more as-1 elements. The distances between as-1 elements in the same promoter were computed and plotted as a histogram binned every 50 bp using R (v.4.1.1).

### F. Gel EMSA

The npr1(dim) mutant (L49D/F53D/V56D/V83K) was cloned into the pTrcHis vector (Thermo Fisher Scientific) as a His10-MBP fusion protein containing a PreScission protease site following MBP and a C-terminal Strep tag, and was expressed and purified similarly to the WT NPR 1 protein. Purified TGA3 (Asn87-Thr384) at a concentration of 0.6 μM and WT NPR1 or the npr1(dim) mutant at a concentration of 8 μM were used in the EMSA with the 6-fluorescein (6-FAM)-labelled oligonucleotide probes (200 nM) containing the LS5 and LS7 as-I cis-elements of the SA-responsive PRI gene promoter (LS5/LS7, 6-FAM-C-gggCTATGACGTAAGTAAAATAGTGACGTAGAGAggg) or DNA sequences with the two as-1 regions replaced with adenosines (LS5/LS7, 6-FAM-C-gggCTAAAAAAAAAGTAAAATAGAAAAAAAAAGAggg), with the LS7 as-1 element replaced with adenosines (LS5/LS7, 6-FAM-C-gggCTATGACGTAAGTAAAATAGAAAAAAAAA-GAggg) or with the LS5 as-1 element replaced with adenosines (LS5/LS7, 6-FAM-C-gggCTAAAAAAAAAGTAAAATAGTGACGTAGAGAggg) as well as their complementary oligonucleotides (Integrated DNA Technologies). The uppercase bases indicate the LS5/LS7 sequence; the lowercase bases indicate overhangs outside the LS5/LS7 sequence; and bases replaced with adenosines are indicated with A's in the small capital font. Pairs of complementary DNA strands were annealed before the binding reaction. For each binding reaction, the desired protein combination was mixed with the target DNA probe together with 30 ng/μL of poly (dI-dC) (non-specific competitor, Thermo Fisher Scientific) in a binding buffer containing 20 mM HEPES (pH 8.0), 300 mM NaCl, 10% glycerol, 1 mM DTT and 0.2 mM SA. The mixture was incubated at 4° C. for 30 min and then run on a 3.8% native polyacrylamide gel in the TB buffer (100 mM Tris-borate pH 8.3, 10% glycerol). After electrophoresis, the gel was scanned with the Typhoon FLA 7000 imager (GE Healthcare).

### G. Plant Material and Transformation

A. thaliana mutants, and transgenic plants are all in the Col-0 ecotype background. Arabidopsis mutant npr1-2 (ref. 6), and transgenic lines expressing NPR1-GFP in the npr1-2 background and npr1(C82A)-GFP in the npr1-1 background were described previously. Transgenic Arabidopsis expressing npr1 (dim)-GFP in the npr1-2 background was generated and two independent lines homozygous for the transgene were used. Seeds were stratified at 4° C. for 3 days before sowing and plants were grown under 12 h-12 h light-dark cycles at 22° C. N. benthamiana plants were grown under the same conditions.

The coding sequences of WT AtNPR1 (AT1G64280) and AtTGA3 (AT1G22070), as well AtNPR1 mutants generated with the QuikChange II site-directed mutagenesis kit (Agilent), were subcloned into the pDONR207 gateway donor vector and confirmed by sequencing. The npr1 mutants were designed based on the structural information. The SBD-ANK cross-linked mutant npr1(Q400C/E401L/R506C) was designed based on the proximity and geometry of Gln400 and Arg506 side chains in the docked conformation of the NPR1 SBD onto ANKs. The E401L mutation was introduced to compensate for the loss of the Glu401-Arg506 hydrogen bond and avoid burial of a charged residue at the protein interface. The obtained entry vectors carrying WT or mutated genes were recombined into the plant binary vectors pK7FWG2, pLN462 and pSITE-4NB to generate C-terminal eGFP, HA and mRFP fusion proteins, respectively. The pPR1: DUAL-LUC reporter was described previously.

For plant transformations, the Agrobacterium tumefaciens strain GV3101 was transformed with plant binary vectors carrying the indicated transgenes. For stable expression in Arabidopsis, a floral dipping method was used. For transient expression in N. benthamiana, the Agrobacterium carrying the indicated construct was cultured over-night at 28° C. in LB broth medium supplemented with appropriate antibiotics: spectinomycin (100 μg ml-1), kanamycin (50 μg ml-1), gentamycin (50 μg ml-1) and rifampicin (25 μg ml-1). The obtained culture was re-inoculated at 1:10 into fresh growth medium with antibiotics and grown for another 4 h. Cells were then centrifuged at 1,600g for 10 min, and inoculum was prepared by resuspending cells to an OD600 of 1 in double-distilled water containing 200 μM acetosyringone (Sigma-Aldrich). The co-expression assays were per-formed with a 1:1 mixture of Agrobacteria inoculums. The inoculum was pressure-infiltrated into N. benthamiana leaves at the abaxial side using 1 ml syringe without the needle.

### H. In Planta Gene Expression Analysis

Expression of the defense genes PRI, WRKY18, WRKY38 and WRKY62 in transgenic Arabidopsis was performed by quantitative PCR (qPCR). Total RNA was extracted from fresh leaf tissue with Trizol reagent (Sigma-Aldrich). cDNA was synthetized using the SuperScript III cDNA Synthesis kit (Thermo Fisher Scientific). qPCR was performed using the FastStart Universal SYBR Green Master Kit (Roche) using the Mastercycler ep realplex (Eppendorf) system. A list of the gene-specific primers used for qPCR is provided in Supplementary Table 1.

The PRI promoter activity was tested using dual-luciferase (DUAL-LUC) reporter assay as described previously. The pPRI: DUAL-LUC reporter was transiently co-expressed in N. benthamiana together with HA-fused NPR1 or its mutants as effectors. The free HA effector was used as a negative control. Plants were treated with 2 mM SA for 24-30 h at 1 day after inoculation. At 2 days after inoculation, leaf discs were collected, ground in liquid nitrogen, and lysed with the passive lysis buffer (PLB) of the Dual-Luciferase Reporter Assay System (Promega, E1910). Lysate was centrifuged at 12,000g for 1 min, and 10 μl was taken for measuring firefly LUC (F-LUC) and Renilla LUC (R-LUC) activities according to the manufacturer's instructions using the Victor3 plate reader (Perkin Elmer). At 25° C., substrates for F-LUC and R-LUC were added using the automatic injector and, after 3 s shaking and 3 s delay, the signals were captured for 3 s and recorded as counts per second. To obtain the PRI promoter activity, the ratio of F-LUC over the R-LUC activities was calculated for each effector.

### I. Protein Analysis

Recombinant protein analysis, co-immunoprecipitation and cell-free oligomerization were performed using total protein extracts from the transient expression assay in N. benthamiana as described previously. Interaction analysis of NPR1-HA with TGA3-mRFP or NPR1-GFP (self-interaction), was performed in vivo by transiently co-expressing the two proteins in N. benthamiana followed by treatment with water (mock) or 1 mM SA for 5 h. Total protein was extracted and used for co-immunoprecipitation with anti-RFP or anti-HA beads, respectively. In the NPR1-HA/TGA3-mRFP interaction experiments the npr1-1-HA was used as a negative control. Interaction between GST-NPR1 and Myc-CUL3A was performed by co-expression in E. coli as described. For western blotting under reducing conditions, the SDS sample buffer was added to the protein extracts from a 4× stock solution supplemented with 50 mM DTT and 715 mM B-mercaptoethanol. For western blotting under non-reducing conditions, a DDT-and B-mercaptoethanol-free SDS sample buffer was used. The blots were probed with anti-GFP (Clontech, 632381, 1:5,000), anti-HA (BioLegend, 901513, 1:1,000), anti-RFP (ChromoTek, 6G6, 1:5,000), anti-cMyc (Santa Cruz, SC-40, 9E10, 1:1,000) or anti-GST-HRP (GE Healthcare, RPN1236V, 1:10,000) primary antibodies. Secondary anti-mouse-HRP antibodies (Abcam, Ab97040) were used at 1:20,000.

### J. NPR1 Oligomerization Assay in Plant Extract

For the cell-free oligomerization assay, total proteins were extracted with lysis buffer (50 mM Tris-HCl pH 7.5, 150 mM NaCl, 0.2% Triton X-100, 0.2% Nonidet P-40, 5% glycerol) supplemented with 1 mM PMSF, 100 μM MG132, EDTA-free protease inhibitor cocktail and 14.3 mM B-mercaptoethanol. The obtained lysate was diluted 10-fold with the lysis buffer supplemented with 1 mM PMSF, 100 μM MG132 and EDTA-free protease inhibitor cocktail. The samples were incubated in the dark at 4° C. for 0, 1, 2 and 3 h time periods before SDS-PAGE analysis under reducing or non-reducing conditions.

### K. Pathogen Infection

For soil-grown Arabidopsis plants, SA treatment was carried out with spray at 1 mM concentration 24 h before gene expression analysis or bacterial infection. For bacterial infection, P. syringae pv. maculicola ES4326 (Psm ES4326) was grown for 2 days on solid King's B medium supplemented with 100 μg/mL streptomycin. An inoculum was prepared by resuspending cells in 10 mM MgSO4 to obtain an OD600 of 0.001. Bacteria were pressure-infiltrated into mature leaves of three-week old Arabidopsis plants and bacterial growth was assessed at 3 days after inoculation by collecting leaf disks from eight infected plants per treatment/genotype, ground in 10 mM MgSO4, and plated at sequential dilutions on King's B medium plates supplemented with 100 μg ml-1 streptomycin. At 2 days after inoculation, the bacterial colonies were scored.

### L. Confocal Imaging

Confocal fluorescence microscopy was performed using the Zeiss 880 airyscan inverted confocal microscope with a ×40/1.2 NA water correction objective. eGFP was excited with a 488 nm argon laser and emission was collected with a 505-530 nm bandpass filter. NPR1-GFP or npr1(dim)-GFP localization was tested in leaf epidermal cells of N. benthamiana after transient expression. To determine nucleocytoplasmic partitioning, the fluorescence intensities from the entire cell (total) and from nuclei were quantified from 15-20 randomly sampled unsaturated confocal images (512×512 px, 225×225 μm) using an automated image analysis algorithm implemented in the ImageJ software as previously described. The obtained intensity values were used to calculate the ratio of nuclear signal over total signal in Microsoft Excel.

### M. Statistics and Reproducibility

Data plotting and statistical tests were performed in GraphPad Prism 8. Statistical parameters such as mean±s.d. and 95% confidence intervals are indicated in the figure legends. In the graphs, asterisks and lowercase letters indicate statistical significance tested either by Stu-dent's t-tests (two groups) or ANOVA (multiple groups) at a significance threshold of P<0.05. The number of biological replicates is indicated for all the data in figure captions. In the figures, the individual datasets (FIGS. 3 (Panel h and j), 4 (Panel d), 5 (Panel g), and 6 (Panels e-h)) and images (FIGS. 3 (Panel i and k), 4 (Panel c and f), 5 (Panel f), and 6 (Panels a and c-e)) are representative of at least two experimental repeats.

The cryo-EM structures of the apo NPR1, NPR12-TGA32 and TGA32-NPR12-TGA32, and X-ray crystal structures of the NPR1 (2SBD) and TGA3 NID have been deposited at the PDB (www.pdb.org) under accession codes 7MK2, 7TAD, 7TAC, 7MK3 and 7TAE, respectively. The cryo-EM density maps of apo NPR1, NPR1-SA, NPR12-TGA32 and TGA32-NPR12-TGA32 have been deposited to the Electron Microscopy Data Bank under accession codes EMD-23884, EMD-23885, EMD-25771 and EMD-25769, respectively. There are no restrictions on data availability. Source data are provided with this paper.

1. A genetically modified plant, wherein the genetically modified plant comprises a nucleic acid encoding a npr1(SAL) protein.

2. The genetically modified plant of claim 1, wherein the nucleic acid comprises a heterologous nucleic acid encoding the npr1(SAL) protein.

3. The genetically modified plant of claim 2, wherein the heterologous nucleic acid encodes an Arabidopsis npr1(SAL) protein or an ortholog of an Arabidopsis npr1(SAL) protein.

4. The genetically modified plant of claim 3, wherein the heterologous nucleic acid:
(a) encodes SEQ ID NO:1 or an ortholog thereof;
(b) encodes any one of SEQ ID NOs: 5-22;
(c) comprises a nucleic acid sequence having at least 75% identity to SEQ ID NO: 2, or an ortholog thereof, and encodes a npr1(SAL) protein; or
(d) comprises SEQ ID NO:2 or an ortholog thereof.

5. The genetically modified plant of claim 4, wherein the ortholog of the Arabidopsis npr1(SAL) protein is derived from a plant of the same species or cultivar as the genetically modified plant.

6. The genetically modified plant of claim 1, wherein the nucleic acid comprises a modified endogenous NPR1 gene, wherein the modified endogenous NPR1 gene is modified to contain a 400C substitution, a 506C substitution, and optionally a 401A, 401L, 402A, or 402L substitution, wherein the positions are relative to the AtNPR1 amino acid sequence (SEQ ID NO:3).

7. The genetically modified plant of claim 6, wherein the endogenous NPR1 gene is modified using CRISPR.

8. The genetically modified plant of claim 1, wherein the genetically modified plant has increased growth under biotic stress or abiotic stress conditions.

9. The genetically modified plant of claim 1, wherein the genetically modified plant:
(a) has increased resistance to pathogen infection; and/or
(b) has increased growth under stress conditions relative to a similar plant overexpressing NPR1.

10. A method of increasing growth of a plant during biotic or abiotic stress, the method comprising: expressing in the plant a nucleic acid encoding a npr1(SAL) protein.

11. The method of claim 10, wherein expressing in the plant the nucleic acid encoding the npr1(SAL) protein comprises:
(a) introducing a heterologous nucleic acid encoding the npr 1 (SAL) protein into a plant cell; or
(b) modifying an endogenous NPR1 gene in a plant cell to encode cysteines at positions 400 and 506, and optionally an alanine or leucine at position 401 and/or 402, wherein the positions are relative to the Arabidopsis NPR1 protein (SEQ ID NO:3).

12. The method of claim 10, wherein expressing in the plant the nucleic acid encoding the npr1(SAL) protein comprises introducing a CRISPR system into a plant cell, wherein the CRISPR system:
(a) introduces into the plant cell's genome a heterologous nucleic acid encoding the npr1(SAL) protein; or
(b) modifies the endogenous NPR1 gene to encode cysteines at positions 400 and 506, and optionally an alanine or leucine at position 401 and/or 402, wherein the positions are relative to the Arabidopsis NPR1 protein (SEQ ID NO:3).

13. The method of claim 11, wherein the heterologous nucleic acid encodes an Arabidopsis npr1(SAL) protein or an ortholog of an Arabidopsis npr1(SAL) protein.

14. The method of claim 13, wherein the heterologous nucleic acid:
(a) encodes SEQ ID NO: 1 or an ortholog thereof;
(b) encodes any one of SEQ ID NOs: 5-22;
(c) comprises a nucleic acid having at least 75% identity to SEQ ID NO:2, or an ortholog thereof, and encodes a npr1(SAL) protein; or
(d) comprises SEQ ID NO:2 or an ortholog thereof.

15. The method of claim 13, wherein the ortholog of the Arabidopsis npr1(SAL) protein is derived from a plant of the same species or cultivar as the plant.

16. The method of claim 10, wherein the plant
(a) has increased resistance to pathogen infection; and/or
(b) has increased growth relative to a similar plant overexpressing NPR1.

17. The method of any one of claim 11, wherein the method further comprises generating one or more regenerants following the introducing the heterologous nucleic acid or the modifying the endogenous NPR1 gene.

18. The method of claim 17, wherein the method further comprises
(a) genotyping one or more regenerants for the presence of the nucleic acid encoding the npr1(SAL) protein; and/or
(b) selecting one or more To plants containing the nucleic acid encoding the npr1(SAL) protein.

19. A nucleic acid encoding a npr1(SAL) protein.

20. The nucleic acid of claim 19, wherein the nucleic acid:
(a) an Arabidopsis npr1(SAL) protein;
(b) an ortholog of the Arabidopsis npr1(SAL) protein;
(c) a sequence having at least 75% identity to SEQ ID NO:2 or an ortholog thereof;
(d) SEQ ID NO: 2 or an ortholog thereof; or
(e) a sequence encoding any one of SEQ ID NOs: 5-22.


SEQUENCE LISTING (55 sequences)

SEQ ID NO: 1 - protein, 593 aa, synthetic construct
  VAR_SEQ 401: note = X is A or L
MDTTIDGFADSYEISSTSFVATDNTDSSIVYLAAEQVLTGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSECADENCCHVACRPAVDFMLEVLYLAFIFKIPELITLYQRHLLDVVDKVVIEDTLVILKLANICGKACMKLLDRCKEIIVKSNVDMVSLEKSLPEELVKEIIDRRKELGLEVPKVKKHVSNVHKALDSDDIELVKLLLKEDHTNLDDACALHFAVAYCNVKTATDLLKLDLADVNHRNPRGYTVLHVAAMRKEPQLILSLLEKGASASEATLEGRTALMIAKQATMAVECNNIPEQCKHSLKGRLCVEILECXDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKCFFPRCSAVLDQIMNCEDLTQLACGEDDTAEKRLQKKQRYMEIQETLKKAFSEDNLELGNSSLTDSTSSTSKSTGGKRSNRKLSHRRR

SEQ ID NO: 2 - DNA, 1779 bp, synthetic construct
  source 1..1779: mol_type = other DNA
atggacaccaccattgatggattcgccgattcttatgaaatcagcagcactagtttcgtcgctaccgataacaccgactcctctattgtttatctggccgccgaacaagtactcaccggacctgatgtatctgctctgcaattgctctccaacagcttcgaatccgtctttgactcgccggatgatttctacagcgacgctaagcttgttctctccgacggccgggaagtttctttccaccggtgcgttttgtcagcgagaagctctttcttcaagagcgctttagccgccgctaagaaggagaaagactccaacaacaccgccgccgtgaagctcgagcttaaggagattgccaaggattacgaagtcggtttcgattcggttgtgactgttttggcttatgtttacagcagcagagtgagaccgccgcctaaaggagtttctgaatgcgcagacgagaattgctgccacgtggcttgccggccggcggtggatttcatgttggaggttctctatttggctttcatcttcaagatccctgaattaattactctctatcagaggcacttattggacgttgtagacaaagttgttatagaggacacattggttatactcaagcttgctaatatatgtggtaaagcttgtatgaagctattggatagatgtaaagagattattgtcaagtctaatgtagatatggttagtcttgaaaagtcattgccggaagagcttgttaaagagataattgatagacgtaaagagcttggtttggaggtacctaaagtaaagaaacatgtctcgaatgtacataaggcacttgactcggatgatattgagttagtcaagttgcttttgaaagaggatcacaccaatctagatgatgcgtgtgctcttcatttcgctgttgcatattgcaatgtgaagaccgcaacagatcttttaaaacttgatcttgccgatgtcaaccataggaatccgaggggatatacggtgcttcatgttgctgcgatgcggaaggagccacaattgatactatctctattggaaaaaggtgcaagtgcatcagaagcaactttggaaggtagaaccgcactcatgatcgcaaaacaagccactatggcggttgaatgtaataatatcccggagcaatgcaagcattctctcaaaggccgactatgtgtagaaatactagagtgyctngacaaacgagaacaaattcctagagatgttcctccctcttttgcagtggcggccgatgaattgaagatgacgctgctcgatcttgaaaatagagttgcacttgctcaacgtctttttccaacggaagcacaagctgcaatggagatcgccgaaatgaagggaacatgtgagttcatagtgactagcctcgagcctgaccgtctcactggtacgaagagaacatcaccgggtgtaaagatagcacctttcagaatcctagaagagcatcaaagtagactaaaagcgctttctaaaaccgtggaactcgggaaatgyttcttcccgcgctgttcggcagtgctcgaccagattatgaactgtgaggacttgactcaactggcttgcggagaagacgacactgctgagaaacgactacaaaagaagcaaaggtacatggaaatacaagagacactaaagaaggcctttagtgaggacaatttggaattaggaaattcgtccctgacagattcgacttcttccacatcgaaatcaaccggtggaaagaggtctaaccgtaaactctctcatcgtcgtcgg

SEQ ID NO: 3 - protein, 593 aa, Arabidopsis thaliana
MDTTIDGFADSYEISSTSFVATDNTDSSIVYLAAEQVLTGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSECADENCCHVACRPAVDFMLEVLYLAFIFKIPELITLYQRHLLDVVDKVVIEDTLVILKLANICGKACMKLLDRCKEIIVKSNVDMVSLEKSLPEELVKEIIDRRKELGLEVPKVKKHVSNVHKALDSDDIELVKLLLKEDHTNLDDACALHFAVAYCNVKTATDLLKLDLADVNHRNPRGYTVLHVAAMRKEPQLILSLLEKGASASEATLEGRTALMIAKQATMAVECNNIPEQCKHSLKGRLCVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFPRCSAVLDQIMNCEDLTQLACGEDDTAEKRLQKKQRYMEIQETLKKAFSEDNLELGNSSLTDSTSSTSKSTGGKRSNRKLSHRRR

SEQ ID NO: 4 - DNA, 1779 bp, Arabidopsis thaliana
  source 1..1779: mol_type = other DNA
atggacaccaccattgatggattcgccgattcttatgaaatcagcagcactagtttcgtcgctaccgataacaccgactcctctattgtttatctggccgccgaacaagtactcaccggacctgatgtatctgctctgcaattgctctccaacagcttcgaatccgtctttgactcgccggatgatttctacagcgacgctaagcttgttctctccgacggccgggaagtttctttccaccggtgcgttttgtcagcgagaagctctttcttcaagagcgctttagccgccgctaagaaggagaaagactccaacaacaccgccgccgtgaagctcgagcttaaggagattgccaaggattacgaagtcggtttcgattcggttgtgactgttttggcttatgtttacagcagcagagtgagaccgccgcctaaaggagtttctgaatgcgcagacgagaattgctgccacgtggcttgccggccggcggtggatttcatgttggaggttctctatttggctttcatcttcaagatccctgaattaattactctctatcagaggcacttattggacgttgtagacaaagttgttatagaggacacattggttatactcaagcttgctaatatatgtggtaaagcttgtatgaagctattggatagatgtaaagagattattgtcaagtctaatgtagatatggttagtcttgaaaagtcattgccggaagagcttgttaaagagataattgatagacgtaaagagcttggtttggaggtacctaaagtaaagaaacatgtctcgaatgtacataaggcacttgactcggatgatattgagttagtcaagttgcttttgaaagaggatcacaccaatctagatgatgcgtgtgctcttcatttcgctgttgcatattgcaatgtgaagaccgcaacagatcttttaaaacttgatcttgccgatgtcaaccataggaatccgaggggatatacggtgcttcatgttgctgcgatgcggaaggagccacaattgatactatctctattggaaaaaggtgcaagtgcatcagaagcaactttggaaggtagaaccgcactcatgatcgcaaaacaagccactatggcggttgaatgtaataatatcccggagcaatgcaagcattctctcaaaggccgactatgtgtagaaatactagagcaagaagacaaacgagaacaaattcctagagatgttcctccctcttttgcagtggcggccgatgaattgaagatgacgctgctcgatcttgaaaatagagttgcacttgctcaacgtctttttccaacggaagcacaagctgcaatggagatcgccgaaatgaagggaacatgtgagttcatagtgactagcctcgagcctgaccgtctcactggtacgaagagaacatcaccgggtgtaaagatagcacctttcagaatcctagaagagcatcaaagtagactaaaagcgctttctaaaaccgtggaactcgggaaacgattcttcccgcgctgttcggcagtgctcgaccagattatgaactgtgaggacttgactcaactggcttgcggagaagacgacactgctgagaaacgactacaaaagaagcaaaggtacatggaaatacaagagacactaaagaaggcctttagtgaggacaatttggaattaggaaattcgtccctgacagattcgacttcttccacatcgaaatcaaccggtggaaagaggtctaaccgtaaactctctcatcgtcgtcgg

SEQ ID NO: 5 - protein, 579 aa, synthetic construct
  VAR_SEQ 398: note = X is P, A, or L
  VAR_SEQ 399: note = X is D, L, or A
METIAGFDDFYEISSTSFLAAPAPTDNSGSSTVYPTELLTRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADDSCCHVACRPAVDFMVEVLYLAFVFQIQELVTMYQRHLLDVVDKVNIEDTLVVLKLANICGKACKKLFDKCREIIVKSNVDVVTLKKSLPENIAKQVIDIRKELGLEVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAECCILEKGKLAAKGGVCVEILKCXXNKREPFPEDVFPSLAVAADEFKIRLIDLENRVQMARCLYPMEAQVAMDFARMKGTREFVVTTATDLHMEPFKFVEMHQSRLTALSKTVEFGKCFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 6 - protein, 579 aa, synthetic construct
  VAR_SEQ 398: note = X is P, A, or L
  VAR_SEQ 399: note = X is D, L, or A
METIARFDDFYEISSTSFPAAPAPTDNSGSSTVYPTELLTRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADDSCCHVACRPAVDFMVEVLYLAFVFQIQELVTMYQRHLLDVVDKVNIEDTLVVLKLANICGKACKKLFDKCREIIVKSNVDVVTLKKSLPENIAKQVIDIRKELGLDVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAECCILEKGKLAAKGGVCVEILKCXXNKREPFPEDVFPSLAVAADEFKIRLIDLENRVQMARCLYPMEAQVAMDFARMKGTREFVVTTATDLHMEPFKFVEMHQSRLTALSKTVEFGKCFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 7 - protein, 579 aa, synthetic construct
  VAR_SEQ 398: note = X is P, A, or L
  VAR_SEQ 399: note = X is D, L, or A
METIAGFDDFYEISSTSFLAAPAPTDNSGSSTVYPTELLTRPEVSAFQLLSTSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADESCCHVACRPAVDFMVEVLYLAFVFQIQELVTMYQRHLLDVVDKVMIEDTLVVLKLANICGKACKKLFDKCREIIVKSNVDVVTLKKSLPENIAKQVIDIRKELGLEVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAECCILEKGKLAAKGGVCVEILKCXXNKREPFPEDVFPSLAVAADEFKIRLIDLENRVQMARCLYPMEAQVAMDFARMKGTREFVVTTATDLHMEPFKFVEMHQSRLTALSKTVEFGKCFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 8 - protein, 581 aa, synthetic construct
  VAR_SEQ 399: note = X is P, A, or L
  VAR_SEQ 400: note = X is D, L, or A
METIAGFDDFYEISSTSFLAAPAPTDNSGSSTVYPTELLTTRPEVSASQLLSNSLESVFDSPEEFYSDAKLVLSDDREVSFHRCILSARSPFFKAELAAAEKVQKSTPVKLELKKLAAEYDVGFDSVVAVLAYVYCGRVRPPPKGVSECADESCCHVACRPAVDFMVEVLYLAFVFQIPELVTMYQRHLLDVIEKVIIEDTLVVLKLANICGKACKKLFDKCKEIIVMSDVDVVTLKKSLPEDVAKQVIDIRKELGLEVAEPEKHVSNIHKALESDDLALVDMLLNEGHTNLDDAYALHFAVAYCDVQTAKDLLELELADVNRRNPRGYTVLHVAAMRKEPTLIALLLTKGANASETSLDGRTALVIAKQVTKATECCILERGKLSAKGGVCVEILKCXXNKREPFPEDVSPSLAVAADELKIRLIDLENRVQMARCLYPMEAQVAMDFARMKGTHEFVVTTTTDLNMEPFKFVEMHRSRLTALSKTVEFGKCFFPRCSKVLDDILNFEDLTILALVEEETPEQRQQKRQRFMEIQEIVRMAFSKDKEDLGKSSLSPSSSSTSKLNGKKRSIAKLSHRRRR

SEQ ID NO: 9 - protein, 582 aa, synthetic construct
  VAR_SEQ 405: note = X IS A OR L
  VAR_SEQ 406: note = X IS E, L, OR A
MEPPTSHVTNAFSDSDSASVEEGGADADADVEALRRLSDNLAAAFRSPEDFAFLADARIAVPGGGGGGGDLLVHRCVLSARSPFLRGVFARRAAAAAGGGGEDGGERLELRELLGGGGEEVEVGYEALRLVLDYLYSGRVGDLPKAACLCVDEDCAHVGCHPAVAFMAQVLFAASTFQVAELTNLFQRRLLDVLDKVEVDNLLLILSVANLCNKSCMKLLERCLDMVVRSNLDMITLEKSLPPDVIKQIIDARLSLGLISPENKGFPNKHVRRIHRALDSDDVELVRMLLTEGQTNLDDAFALHYAVEHCDSKITTELLDLALADVNHRNPRGYTVLHIAARRREPKIIVSLLTKGARPADVTFDGRKAVQISKRLTKQGDYFGVTEEGKPSPKDRLCIEILECXXRRDPQLGEASVSLAMAGESLRGRLLYLENRVALARIMFPMEARVAMDIAQVDGTLEFNLGSGANPPPERQRTTVDLNESPFIMKEEHLARMTALSKTVELGKCFFPRCSNVLDKIMDDETDPVSLGRDTSAEKRKRFHDLQDVLQKAFHEDKEENDRSGLSSSSSSTSIGAIRPRR

SEQ ID NO: 10 - protein, 588 aa, synthetic construct
  VAR_SEQ 400: note = X is A or L
  VAR_SEQ 401: note = X is E, L, or A
MDNSRTAFSDSNDISGSSSICCIGGGMTESFSPETSPAEITSLKRLSETLESIFDAASPEFDYFADAKLVIPGAGKEIPVHRCILSARSPFFKNLFCGKKEKNSSKVELKEVMKEYEVSYDAVVSVLAYLYSGKIRPSPKDVCVCVDNECSHVACRPAVAFLVEVLYISFTFQISELVDKFQRHLLDILGKAAADDVMMVLSVANICGKACERLLSSCIEIIVKSNVDIITLDKALPHDIVKQITDSRAELGLQGPESNGFPDKHVKRIHRALDSDDVELLQMLLREGHTTLDDAYALHYAVAYCDAKTTAELLDLALADVNHQNSRGYTVLHVAAMRKEPKIIVSLLTKGARPSDLTSDGRKALQIAKRLTRLVDFSKSPEEGKSASKDRLCIEILECXXRRDPLLGEASVSLAMAGDDLRMKLLYLENRVGLAKLLFPMEAKVAMDIAQVDGTSEFPLASISKKMVNAQRTTVDLNEAPFRIKEEHLNRLRALSRTVELGKCFFPRCSEVLNKIMDADDLSEIAYMGNDTAEERQLKKQRYMELQEILTKAFTEDKEEFDKTNNISSSCSSTSKGVDKPNKLPFRK

SEQ ID NO: 11 - protein, 582 aa, synthetic construct
  VAR_SEQ 393: note = X is A or L
  VAR_SEQ 394: note = X is E, L, or A
MNFRSGSSDSKDASNSSTGEAYLSGVSDVITPLRRLSEQLGSILDGGGVDFFSDAKIVAGDGREVAVNRCILAARSGFFKHVFAGGGGCVLRLKEVAKDYNVGLEALGIVLAYLYSGRVKPLPQGGVCVCVDDVCSHFGCRPAIDFLLQLLYASSTFQLNDSIQQHILFAFCSNAGHLLDILEKVAIDDILVVLSVANICGIVCERLLARCTEMILKSDADITTLEKALPQHLVKQITDKRIELDLYMPENFNFPDKHVNRIHRALDSDDVELVRLLLKEGHTTLDDAYALHYAVAYCDVKTTTELLDLGLADVNHKNYRGYSVLHVAAMRKEPKIIVSLLTKGAQPSDLTLDGRKALQISKRLTKAVDYYKSTEEGKVSCSDRLCIEILECXXRREPLLGEASLSLAMAGDDLRMKLLYLENRVGLAKVLFPMEAKVIMDISQIDGTSEFPSTDMYCPNISDHQRTTVDLNDAPFRMKEEHLVRLRALSRTVELGKCFFPRCSEVLNKIMDADDLTQLTCMGDDSPEDRLRKRRRYVELQEVLNKVFNEDKEEFDRSAMSSSSSSTSIGVVRPNANLAMKN

SEQ ID NO: 12 - protein, 589 aa, synthetic construct
  VAR_SEQ 397: note = X is A or L
  VAR_SEQ 398: note = X is E, L, or A
MDNRIGFSDSNEISNGSSTCCIETPSTSKPFTNPEIVALQQLSGNLEAIFDSQDFDYFADAKITSSNYNREVPVHRCILSARSPFFKSVFSSPVAKDRSGVAKFELKELAKDYDVGFDSLMTVLGYLYCGKVRPWPKDVCACVDDDCSHIACRPAVDLLTEVLYASFTFQVNELVALYQRHLLDILDKVSTDDILVILAVANICGEACERLLTRCVEIIVKSNVDIVTLDKALPQYIVKKIMDSRLELGLNVPENSNLLDKHVKRIHRALDSDDVELVRMLLKEAHTNLDDAHALHYAVSYCDAKTTTEILDLGLADVNCRNSRGYTVLHVAAMRKDPKIIVSLLTKGARLSDLTLDGRKALQISKRLTRAMDYHKSTEEGKASPKERLCIEILECXXRRDPLLGEASLSLAMAGDDLRMKLLYLENRVGLAKLLFPMEAKVAMDIAQVDGTSEFPLAGIRPSILSGAQRGAVDLNEAPFRMHEEHLNRMRALSRTVELGKCFFPRCSDVLNKIMDADDLSQIAYLGNETSEERLVKRQRHLELQDALSKAFNEDKQEFDRSVISSSSSTKSIGTARSNGKLIDMGGGH

SEQ ID NO: 13 - protein, 586 aa, synthetic construct
  VAR_SEQ 387: note = X is A or L
MADSHEPSSSLSFTSSSYTNGSQSCNMSPSSISDPMPSLEVISLNKLSSNLAQLLIHDGCDYTDADIVVEGVPVGIHRCILAVRSRFFHYLFQKDEKPAMKDGKPQYHMNELLPYGKVGHEAFLILLSYLYSGKLNSSPANVSTCVDNSCAHDACGPAIDFAVQLTYASSIFQIPELVSLFQRHLLNYVVKALVENVIQILVVAFHCQLSPLVTQCIDRIARSDLDCASLEKGLPYEVAERIKLVRLKSRGGDEQNLVADSPRDKKIKKICLALDSDDVELMKLLLSESDVTLDEANALHYAAAYCDPKSLTEVLNLNIADVNLRNSRGYTVLHVAAMRKDPSVIMSLLNKGAWAFDLTPDGRTAANICQRLTRPKDYHAKTEKGCXTNKDRLCIDILEREMWRNPTSDSSILSLAMADDVHMKLIYLENRVAFARLLFPSEARLAMDIANADTTSEFVGLSMPKNSNKNLREVNLNETPSVQNKRFLSRMQALLKTVEMGRCFFPNCSEALDKFVADDLPDLFYLEKGTVEEQRIKRKRFKELKNDVQKAFDKDKAAKLNQSGLSPSSSSTSLKHGTNHRNVRRQ

SEQ ID NO: 14 - protein, 583 aa, synthetic construct
  VAR_SEQ 403: note = X is A or L
  VAR_SEQ 404: note = X is E, L, or A
MEPMDSQLTALALSDSDSASVEGAAADAADADLQALRRLSDNLAAAFRSPDDFAFLADARIVVPGAPDLRVHRCVLCARSPFLRDAFARRAASAGEEEKDKDSYMCKVELRDLLGDEVEVGYDALRLVLDYLYSGRVAALPKAACLCVDEDACAHVGCRPAVAFMAQVLFAASTFDVAELTNLFQRRLLDVLDKVEVDNLPLVLSVANLCSKSCVKLLERCLDVVVRSNLDMIALEKKLPPDVVKEIVDARVSLGLVSPEDKGFPNIHVRRIHRALDSDDVELVRMLLKEGKTNLDDAYALHYAVEHCDSKITTELLDLALADVNHRNPRGYTVLHIAAMRREPKIIVSLLTKGARPSDLTFDDRKAVQISKRLTKHGDYFGPTEDGKPSPKDRLCIEVLECXXRRDPQLGEASVSLAIEGDSARGRLLYLENRVALARILFPMEARVAMDIAQVDGTLEFTLVSSVNLPAEIQRTVDLNDTPFTMKEEHLARMRALSKTVEVGKCFFPRCSKVLDTIMDDEAEMASLGRDTSAEKKRRFHDLQDLVQKAFSEDKEENDRSAARSPSSSSTTTTSIGAVRPRR

SEQ ID NO: 15 - protein, 576 aa, synthetic construct
  VAR_SEQ 398: note = X is A or L
  VAR_SEQ 399: note = X is E, L, or A
MDSRTAFSDSNDISGSSSICCMNESETSLADVNSLKRLSETLESIFDASAPDFDFFADAKLLAPGGKEIPVHRCILSARSPFFKNVFCGKDSNTKLELKELMKEYEVSFDAVVSVLAYLYSGKVRPASKDVCVCVDNECLHVACRPAVAFMVQVLYASFTFQISQLVDKFQRHLLDILDKAVADDVMMVLSVANICGKACERLLSRCIDIIVKSNVDIITLDKSLPHDIVKQITDSRAELGLQGPESNGFPDKHVKRIHRALDSDDVELLRMLLKEGHTTLDDAYALHYAVAYCDAKTTAELLDLSLADVNHQNPRGHTVLHVAAMRKEPKIIVSLLTKGARPSDLTSDGKKALQIAKRLTRLVDFTKSTEEGKSAPKDRLCIEILECXXRRDPLLGEASLSLAMAGDDLRMKLLYLENRVGLAKLLFPMEAKVAMDIAQVDGTSELPLASMRKKIADAQRTTVDLNEAPFKMKEEHLNRLRALSRTVELGKCFFPRCSEVLNKIMDADDLSEIAYMGNDTVEERQLKKQRYMELQEILSKAFTEDKEEFAKTNMSSSCSSTSKGVDKPNNLPFRK

SEQ ID NO: 16 - protein, 584 aa, synthetic construct
  VAR_SEQ 395: note = X is A or L
  VAR_SEQ 396: note = X is E, L, or A
MDYRAALSDSNDFSGSSSICCIAATTESLSSEVSPPDISALRRLSENLESVFESPEFDFFTDARIVVAGGREVPVHRCILAARSVFFKAVLAGARKEKEAKFLLKDLAKEFDVGYDSLVAVLGYLYSGRVGALPKGVCACVDDDCPHSACRPAVDFMVEVLYASFAFQISELVGLYQRRLMDILDKVASDDILVILSVANLCGKACDRLLARCIDIIIKSDVDVVTLERALPQEMVKQIVDSRLELGFEEPESTNFPDKHVKRIHRALDSDDVELVRMLLKEGHTTLDDAYALHYAVAFGDAKTTTELLDLGLADVNHKNHRGYTVLHIAAMRKEPKIIVSLLTKGARPTDITPDGRNALQIAKRLTRAVDYHKSTEEGKPSPKDQLCVEVLECXXRRDPLIGEASFSLAIAGDDLRMKLLYLENRVGLAKLLFPMEAKVAMDIAQVDGTSEFTLTAIRPRNLADAQRTTVDLNEAPFRIKEEHLNRLRALSKTVDLGKCFFPRCSEVLNKIMDADDLSDLAYLGNGTTEERLLKKRRYKELQDQLCKAFNEDKEENDKSRISSSSSSTSLGFGRTNSRLSCKK

SEQ ID NO: 17 - protein, 576 aa, synthetic construct
  VAR_SEQ 396: note = X is A or L
  VAR_SEQ 397: note = X is E, L, or A
MEAPSSHVTTSFSDCDSVSMEDAAPDADVEALRRLSDNLAAAFRSPDDFAFLADARFAVPGAPDLCVHRCVLSARSPFLRALFKRRAAAAGSAGGAEGDRVELRELLGGEVEVGYEALRLVLDYLYSGRVCDLPKTACACVDEGGCAHVGCHPAVSFMAQVLFAASTFQVGELASLFQRHLLDLLDKVEADNLPLVLSVANLCNKSCVKLFERCLERVVRSDLDMITLDKALPLDVIKQIIDSRITLGLASPEDNGFPNKHVRRILSALDSDDVELVRLLLKEGQTNLDDAFALHYAVEHCDSKITTELLDIALADVNLRNPRGYTVLHIAARRRDPKIVVSLLTKGARPSDFTFDGRKAVQIAKRLTKHGDYFGNTEEGKPSPNDKLCIEILECXXRRDPQLGEASVSLALAGDCLRGKLLYLENRVALARIMFPIEARVAMDIAQVDGTLEFTLGSCTNPPPEITTVDLNDTPFKMKDEHLARMRALSKTVELGKCFFPRCSNVLDKIMDDEPELASLGRDASSERKRRFHDLHDTLLKAFSEDKEEFARSATLSASSSSTPTVARNLTGRPRR

SEQ ID NO: 18 - protein, 584 aa, synthetic construct
  VAR_SEQ 400: note = X is A or L
  VAR_SEQ 401: note = X is E, L, or A
MMYLRSGFSEYSNEISNNTSSELCCTTAPNSTITASQDVITFTQPLNRLSDNLASILDDTGFDFFSDAKIIAKDGREVSVHRCILSARSSFFKDVFKGKKETTLQLKEVAKDYDVGFDALNVVLRYLYSERVEDHHLSAKDVCVCVDDDCLHFGCWPVVDFMLQLLYASFTFQISELLALYQDHLLDILDKMAIDDMLVVLSIANICGKTCDKLLKRCTDIIVESNVDITTLEKSLPQSIVKLVTYKRKQLGLDMYETVNLLDKHVTRIHRALDSDDVELVRLLLKEGHTTLDEAHALHYAVAYCDVKTTTELLDLGLADVNHKNLRGYSVLHVAAKRKEPKIIVSLLTKGAQPSELTMDGRKALQISKRCTKAVDYYKSTEEGKVSSNDRLCIEILECXXRREPLHGEASLSLAKAGDDLRMKLLYLENRVGLAKLLFPMEAKVVMDITPIDGTSEFTPNLGGYQRTTMDLNEAPFKIKEEHLIRMKALSRAVELGKCFFPRCSEVLNKIMDADDLSQLACMGHDSPEDRQVKRRRYAELQEVLNKVFHEDKEEFDKSGMSSSSSSTSIGMPRANNSMIAMNH

SEQ ID NO: 19 - protein, 583 aa, synthetic construct
  VAR_SEQ 403: note = X is A or L
  VAR_SEQ 404: note = X is E, L, or A
MEPMESQLTAVALSDSDSASVEGAAAADAADADLQALRRLSDNLAAAFRSPDDYGFLSDARIAVPGAPDLRVHRCVLCARSPFLRDVFARRAASDGEEKDKGRDRGKVELRDLLGDEVEVGYEALRLVVDYLYSGRVAALPKAACLCVDEDGCAHVGCRPAVAFMAQVLFAASTFDVAELTNLFQRRLLDVLDKVEVDNLPLILSVANLCNKSCVKLLERCLDVVVRSNLDMITLEKALPPDVVKAIVDARLSLGLVLPEDKGFPNIHVRRVHRALDSDDVELVRMLLKEGKTNLDDAYALHYAVEHCDSKITTELLDFALADVNHRNPRGYTVLHIAAMRREPKIIVSLLTKGARPSDLTFDHRKAVQISKRLTNHGDYFGPTEDGKPSPKDRLCIEILECXXRRDPHLGEASVSLAIEGDSARGRLLYLENRVALARILFPMEARVAMDIAQVDGTLEFTLGSNANLPTEIQRTVDLNDTPFIMKEEHLARMRALAKTVELGKCFFPRCSKVLDKIMDDETELASLGRDTSTEKKRRFHDLQDLVHKAFSEDKEENDRSAARSSSSSSTTSIGIGAVRPRR

SEQ ID NO: 20 - protein, 577 aa, synthetic construct
  VAR_SEQ 397: note = X is A or L
  VAR_SEQ 398: note = X is E, L, or A
MEAPSSHVTASFSDCDDSVSMEDAAPDADVEALRRLSDNLAAAFRSPDDFAFLADALVAVPGAPDLRVHRCVLSARSPFLRALFKRRAAAAGSSGGVEGNRLELRELLGDEVEVRYEALELVLDYLYSGRVRDLPKSACACVDEGGCAHVGCHPAVSFMAQVLFAASTFQVGELANLFQRHLLDFLDKVEVDNLPLILSVANLCNKSCVKLFERCMEMVVRSNLDMITLEKALPQDVIKQITDLRITLGLASPEDNGFPNKHVRRILRALDSDDVELVRMLLTEGQTNLDDAFALHYAVEHCDSKITTELLDIALADVNLRNPRGYTVLHIAAKRRDPKIVVSLLTKGARPSDFTFDGRKAVQISKRLTKHGDYFGNTEEGKPSPNDKLCIEILECXXRRDPQLGEASLSLALAGDCLRGKLLYLENRVALARIMFPIEARVAMDIAQVDGTLEFTLGSSTNPPLEITTVDLNDTSFKMKEEHLARMRALSKTVELGKCFFPRCSNVLDKIMDDEPELASLGRDASSERKRRFHDLQDTLLKAFSEDKEEFNRTTTLSSSSSSTSTVARNLAGRTRR

SEQ ID NO: 21 - protein, 585 aa, synthetic construct
  VAR_SEQ 399: note = X is A or L
  VAR_SEQ 400: note = X is E, L, or A
MDNRNGFSDSNEISNNSRTSCVAAAANTESFYSSEPVNSDITALRILSKTLETIFESQDFDYFTDAKIVLSTGREVPVHRCILSSRSGFFKNVFAGTGKQRGPKFELKELVRDYEVGFDPLVAVLAYLYCGKVRPFPIGVCVCVDDDACSHVACRPAVDFMVEVLYVSFAFQVPELVALYQRHLLDILDKVVADDILVVLSVAHMCGKACEKLLERCIEITVKSDIDIVTLDKTLPQHIVKQIIDLRVELSLHRSESCGFPDKHTKRIHRALDSDDVELVRMLLKEAHTNLDDAHALHYAVAYCDAKTTTELLDLGLADVNHRNSRGYTVLHVAAMRKEPKIIVSLLTKGARPSDLTLDGRKALQISKRLTKAADYYIPTEEGKTTPKDRLCIEILECXXRRDPLLREASHSFAMAGDDLRMKLLYLENRVGLAKLLFPMEAKVIMDIVHLDGTLEFALDGIKTKKMAGAQRTTVDLNEAPFKMQEEHLNRMKALCRTVELGKCFFPRCSEVLNKIMDADDLNQLACPGNDTPEERLLKRIRYMELQEVVSKAFNEDKEEFDRSAISSSSSSKSVVRPRGGKRTH

SEQ ID NO: 22 - protein, 585 aa, synthetic construct
  VAR_SEQ 398: note = X is A or L
  VAR_SEQ 399: note = X is E, L, or A
MEYTKGCVSDSNEVVSSSISTLAHHHSPSSSSPPPDITSLKHLSNNLELMVNSSDFDFFADAKIVVSNGREIHVHRCILAARSSFFRNVFSAKEGSSSTRFELKNLAKEYDVGLDSLLAVLGYLYSGKLKSLPKGVCVCVDDACSHVACRPVVDFMVEVLYASSTFQISELVTLYQKCLLDILEKTAIDDVLVVLHVANMCGKACEGLVRRCIEMIVKSDVDIVTLEKALPQQSVKRITDSRLALGLDSSRSSNSPSNFPDKHTRRIHRALDSDDVELVRMLLKEGHTNLDEAYALHYAVAYCDAKTTYELLDLGIADVNCRNLRGYTVLHLAAARKEPKIIVSLLTKGARPSDLTLDGRKALQITKRLTRAADFFKSNDEGKACPNDRLCIEILECXXRRDPLLGEASLSLAMAGDDLRMKLLYLENRVGLARYLFPMEAKVVLGITQLDETPAFPVACLIRNNPTGSRTTTVDLNEAPFKIKEEHLARLRALCQSVELGKCFFPRCSEVLNKIMDGDDMSQLMYTGDDTPEVRLTKRKRYMEIQEVLSKAFDEDKQDIDRSGMSSSSSSTSVRAVRPKLTVID

SEQ ID NO: 23 - protein, 157 aa, Arabidopsis thaliana
ASASEATLEGRTALMIAKQATMAVECNNIPEQCKHSLKGRLCVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFP

SEQ ID NO: 24 - protein, 145 aa, Brassica juncea
ANALETSLDGRTALLIAKQVTKAAECCILEKGKLAAKGGVCVEILKQPDNKREPFPEDVFPSLAVAADEFKIRLIDLENRVQMARCLYPMEAQVAMDFARMKGTREFVVTTATDLHMEPFKFVEMHQSRLTALSKTVEFGKRFFP

SEQ ID NO: 25 - protein, 154 aa, Triticum sp.
ARPSDFTFDGRKAVQISKRLTKHGDYFGNTEEGKPSPNDKLCIEILEQAERRDPQLGEASLSLALAGDCLRGKLLYLENRVALARIMFPIEARVAMDIAQVDGTLEFTLGSSTNPPLEITTVDLNDTSFKMKEEHLARMRALSKTVELGKRFFP

SEQ ID NO: 26 - protein, 154 aa, Hordeum vulgare
ARPSDFTFDGRKAVQIAKRLTKHGDYFGNTEEGKPSPNDKLCIEILEQAERRDPQLGEASVSLALAGDCLRGKLLYLENRVALARIMFPIEARVAMDIAQVDGTLEFTLGSCTNPPPEITTVDLNDTPFKMKDEHLARMRALSKTVELGKRFFP

SEQ ID NO: 27 - protein, 156 aa, Oryza sativa
ARPADVTFDGRKAVQISKRLTKQGDYFGVTEEGKPSPKDRLCIEILEQAERRDPQLGEASVSLAMAGESLRGRLLYLENRVALARIMFPMEARVAMDIAQVDGTLEFNLGSGANPPPERQRTTVDLNESPFIMKEEHLARMTALSKTVELGKRFFP

SEQ ID NO: 28 - protein, 155 aa, Zea mays
ARPSDLTFDDRKAVQISKRLTKHGDYFGPTEDGKPSPKDRLCIEVLEQAERRDPQLGEASVSLAIEGDSARGRLLYLENRVALARILFPMEARVAMDIAQVDGTLEFTLVSSVNLPAEIQRTVDLNDTPFTMKEEHLARMRALSKTVEVGKRFFP

SEQ ID NO: 29 - protein, 155 aa, Sorghum bicolor
ARPSDLTFDHRKAVQISKRLTNHGDYFGPTEDGKPSPKDRLCIEILEQAERRDPHLGEASVSLAIEGDSARGRLLYLENRVALARILFPMEARVAMDIAQVDGTLEFTLGSNANLPTEIQRTVDLNDTPFIMKEEHLARMRALAKTVELGKRFFP

SEQ ID NO: 30 - protein, 157 aa, Fragaria sp.
ARPSDLTLDGRKALQITKRLTRAADFFKSNDEGKACPNDRLCIEILEQAERRDPLLGEASLSLAMAGDDLRMKLLYLENRVGLARYLFPMEAKVVLGITQLDETPAFPVACLIRNNPTGSRTTTVDLNEAPFKIKEEHLARLRALCQSVELGKRFFP

SEQ ID NO: 31 - protein, 157 aa, Glycine max
AQPSDLTLDGRKALQISKRLTKAVDYYKSTEEGKVSCSDRLCIEILEQAERREPLLGEASLSLAMAGDDLRMKLLYLENRVGLAKVLFPMEAKVIMDISQIDGTSEFPSTDMYCPNISDHQRTTVDLNDAPFRMKEEHLVRLRALSRTVELGKRFFP

SEQ ID NO: 32 - protein, 157 aa, Citrus sp.
ARPSDLTLDGRKALQISKRLTKAADYYIPTEEGKTTPKDRLCIEILEQAERRDPLLREASHSFAMAGDDLRMKLLYLENRVGLAKLLFPMEAKVIMDIVHLDGTLEFALDGIKTKKMAGAQRTTVDLNEAPFKMQEEHLNRMKALCRTVELGKRFFP

SEQ ID NO: 33 - protein, 157 aa, Citrus sp.; identical to SEQ ID NO: 32

SEQ ID NO: 34 - protein, 156 aa, Solanum lycopersicum
ARPSDLTSDGKKALQIAKRLTRLVDFTKSTEEGKSAPKDRLCIEILEQAERRDPLLGEASLSLAMAGDDLRMKLLYLENRVGLAKLLFPMEAKVAMDIAQVDGTSELPLASMRKKIADAQRTTVDLNEAPFKMKEEHLNRLRALSRTVELGKRFFP

SEQ ID NO: 35 - protein, 157 aa, Vitis vinifera
ARPTDITPDGRNALQIAKRLTRAVDYHKSTEEGKPSPKDQLCVEVLEQAERRDPLIGEASFSLAIAGDDLRMKLLYLENRVGLAKLLFPMEAKVAMDIAQVDGTSEFTLTAIRPRNLADAQRTTVDLNEAPFRIKEEHLNRLRALSKTVDLGKRFFP

SEQ ID NO: 36 - protein, 146 aa, Raphanus sativus
GANASETSLDGRTALVIAKQVTKATECCILERGKLSAKGGVCVEILKQPDNKREPFPEDVSPSLAVAADELKIRLIDLENRVQMARCLYPMEAQVAMDFARMKGTHEFVVTTTTDLNMEPFKFVEMHRSRLTALSKTVEFGKRFFP

SEQ ID NO: 37 - protein, 156 aa, Nicotiana tabacum
ARPSDLTSDGRKALQIAKRLTRLVDFSKSPEEGKSASKDRLCIEILEQAERRDPLLGEASVSLAMAGDDLRMKLLYLENRVGLAKLLFPMEAKVAMDIAQVDGTSEFPLASISKKMVNAQRTTVDLNEAPFRIKEEHLNRLRALSRTVELGKRFFP

SEQ ID NO: 38 - protein, 157 aa, Populus trichocarpa
ARLSDLTLDGRKALQISKRLTRAMDYHKSTEEGKASPKERLCIEILEQAERRDPLLGEASLSLAMAGDDLRMKLLYLENRVGLAKLLFPMEAKVAMDIAQVDGTSEFPLAGIRPSILSGAQRGAVDLNEAPFRMHEEHLNRMRALSRTVELGKRFFP

SEQ ID NO: 39 - protein, 168 aa, Cucumis melo
RKDPSVIMSLLNKGAWAFDLTPDGRTAANICQRLTRPKDYHAKTEKGQATNKDRLCIDILEREMWRNPTSDSSILSLAMADDVHMKLIYLENRVAFARLLFPSEARLAMDIANADTTSEFVGLSMPKNSNKNLREVNLNETPSVQNKRFLSRMQALLKTVEMGRRFFP

SEQ ID NO: 40 - protein, 151 aa, Medicago truncatula
AQPSELTMDGRKALQISKRCTKAVDYYKSTEEGKVSSNDRLCIEILEQAERREPLHGEASLSLAKAGDDLRMKLLYLENRVGLAKLLFPMEAKVVMDITPIDGTSEFTPNLGGYQRTTMDLNEAPFKIKEEHLIRMKALSRAVELGKRFFP

SEQ ID NO: 41 - protein, 160 aa, Arabidopsis thaliana
TGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSECADENCCHVACRPAVDFMLEVLYLAFIFKIPELITLYQRHLLDVVDKVV

SEQ ID NO: 42 - protein, 157 aa, Brassica rapa
TRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADDSCCHVACRPAVDFMVEVLYLAFVFQIQELVTMYQRHLLDVVDKVN

SEQ ID NO: 43 - protein, 157 aa, Brassica juncea; identical to SEQ ID NO: 42

SEQ ID NO: 44 - protein, 157 aa, Brassica napus
TRPEVSAFQLLSTSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADESCCHVACRPAVDFMVEVLYLAFVFQIQELVTMYQRHLLDVVDKVM

SEQ ID NO: 45 - protein, 157 aa, Raphanus sativus
TRPEVSASQLLSNSLESVFDSPEEFYSDAKLVLSDDREVSFHRCILSARSPFFKAELAAAEKVQKSTPVKLELKKLAAEYDVGFDSVVAVLAYVYCGRVRPPPKGVSECADESCCHVACRPAVDFMVEVLYLAFVFQIPELVTMYQRHLLDVIEKVI

SEQ ID NO: 46 - skipped

SEQ ID NO: 47 - protein, 172 aa, Oryza sativa
ADADVEALRRLSDNLAAAFRSPEDFAFLADARIAVPGGGGGGGDLLVHRCVLSARSPFLRGVFARRAAAAAGGGGEDGGERLELRELLGGGGEEVEVGYEALRLVLDYLYSGRVGDLPKAACLCVDEDCAHVGCHPAVAFMAQVLFAASTFQVAELTNLFQRRLLDVLDKVE

SEQ ID NO: 48 - protein, 158 aa, Nicotiana tabacum
SPAEITSLKRLSETLESIFDAASPEFDYFADAKLVIPGAGKEIPVHRCILSARSPFFKNLFCGKKEKNSSKVELKEVMKEYEVSYDAVVSVLAYLYSGKIRPSPKDVCVCVDNECSHVACRPAVAFLVEVLYISFTFQISELVDKFQRHLLDILGKAA

SEQ ID NO: 49 - protein, 159 aa, Populus trichocarpa
TNPEIVALQQLSGNLEAIFDSQDFDYFADAKITSSNYNREVPVHRCILSARSPFFKSVFSSPVAKDRSGVAKFELKELAKDYDVGFDSLMTVLGYLYCGKVRPWPKDVCACVDDDCSHIACRPAVDLLTEVLYASFTFQVNELVALYQRHLLDILDKVS

SEQ ID NO: 50 - protein, 168 aa, Zea mays
ADADLQALRRLSDNLAAAFRSPDDFAFLADARIVVPGAPDLRVHRCVLCARSPFLRDAFARRAASAGEEEKDKDSYMCKVELRDLLGDEVEVGYDALRLVLDYLYSGRVAALPKAACLCVDEDACAHVGCRPAVAFMAQVLFAASTFDVAELTNLFQRRLLDVLDKVE

SEQ ID NO: 51 - protein, 155 aa, Solanum lycopersicum
SLADVNSLKRLSETLESIFDASAPDFDFFADAKLLAPGGKEIPVHRCILSARSPFFKNVFCGKDSNTKLELKELMKEYEVSFDAVVSVLAYLYSGKVRPASKDVCVCVDNECLHVACRPAVAFMVQVLYASFTFQISQLVDKFQRHLLDILDKAV

SEQ ID NO: 52 - protein, 155 aa, Vitis vinifera
SPPDISALRRLSENLESVFESPEFDFFTDARIVVAGGREVPVHRCILAARSVFFKAVLAGARKEKEAKFLLKDLAKEFDVGYDSLVAVLGYLYSGRVGALPKGVCACVDDDCPHSACRPAVDFMVEVLYASFAFQISELVGLYQRRLMDILDKVA

SEQ ID NO: 53 - protein, 165 aa, Hordeum vulgare
PDADVEALRRLSDNLAAAFRSPDDFAFLADARFAVPGAPDLCVHRCVLSARSPFLRALFKRRAAAAGSAGGAEGDRVELRELLGGEVEVGYEALRLVLDYLYSGRVCDLPKTACACVDEGGCAHVGCHPAVSFMAQVLFAASTFQVGELASLFQRHLLDLLDKVE

SEQ ID NO: 54 - protein, 155 aa, Medicago truncatula
VITFTQPLNRLSDNLASILDDTGFDFFSDAKIIAKDGREVSVHRCILSARSSFFKDVFKGKKETTLQLKEVAKDYDVGFDALNVVLRYLYSERVEDHHLSAKDVCVCVDDDCLHFGCWPVVDFMLQLLYASFTFQISELLALYQDHLLDILDKMA

SEQ ID NO: 55 - protein, 161 aa, Glycine max
VSDVITPLRRLSEQLGSILDGGGVDFFSDAKIVAGDGREVAVNRCILAARSGFFKHVFAGGGGCVLRLKEVAKDYNVGLEALGIVLAYLYSGRVKPLPQGGVCVCVDDVCSHFGCRPAIDFLLQLLYASSTFQLNDSIQQHILFAFCSNAGHLLDILEKVA